SEQUENCE LISTING (299 sequences)

SEQ ID NO: 1 - protein, 39 aa, Heloderma horridum
HSDGTFTSDLSKQMEEEAVRLFIEWLKNGGPSSGAPPPS

SEQ ID NO: 2 - protein, 39 aa, Heloderma suspectum
HGEGTFTSDLSKQMEEEAVRLFIEWLKNGGPSSGAPPPS

SEQ ID NO: 3 - protein, 39 aa, Artificial Sequence
  Description of Artificial Sequence Synthetic construct
XXXGTXXXXXSKQXEEEAVRLXXXXLKNGGXSSGAXXXX

SEQ ID NO: 4 - protein, 38 aa, Artificial Sequence
  Description of Artificial Sequence Synthetic construct
XXXGXXXXXXXXXXXXXAXXXXXXXXXXGGXSSGAXXX

SEQ ID NO: 5 - protein, 39 aa, Artificial Sequence
  Description of Artificial Sequence Synthetic construct
XXXXXXXXXXXXXXXXXAXXXXXXXXXXGGXSSGAXXXX

SEQ ID NO: 6 - protein, 30 aa, Artificial Sequence
  Description of Artificial Sequence Synthetic construct
HGEGTFTSDLSKQMEEEAVRLFIEWLKNGG

SEQ ID NO: 7 - protein, 30 aa, Artificial Sequence; identical to SEQ ID NO: 6
  Description of Artificial Sequence Synthetic construct

SEQ ID NO: 8 - protein, 28 aa, Artificial Sequence
  Description of Artificial Sequence Synthetic construct
HGEGTFTSDLSKQLEEEAVRLAIEFLKN

SEQ ID NO: 9 - protein, 39 aa, Artificial Sequence
  Description of Artificial Sequence Synthetic construct
HGEGTFTSDLSKQLEEEAVRLFIEFLKNGGPSSGAPPPS

SEQ ID NO: 10 - protein, 39 aa, Artificial Sequence
  Description of Artificial Sequence Synthetic construct
HGEGTFTSDLSKQLEEEAVRLFIEWLKNGGPSSGAPPPS

SEQ ID NO: 11 - protein, 39 aa, Artificial Sequence
  Description of Artificial Sequence Synthetic construct
HGEGTFTSDLSKQMEEEAVRLFIEFLKNGGPSSGAPPPS

SEQ ID NO: 12 - protein, 39 aa, Artificial Sequence
  Description of Artificial Sequence Synthetic construct
YGEGTFTSDLSKQMEEEAVRLFIEWLKNGGPSSGAPPPS

SEQ ID NO: 13 - protein, 39 aa, Artificial Sequence
  Description of Artificial Sequence Synthetic construct
HGEGTFTSDLSKQMEEEAVRLFIEWLKNGGPSSGAPPPY

SEQ ID NO: 14 - protein, 39 aa, Artificial Sequence
  Description of Artificial Sequence Synthetic construct
HGDGTFTSDLSKQMEEEAVRLFIEWLKNGGPSSGAPPPS

SEQ ID NO: 15 - protein, 39 aa, Artificial Sequence
  Description of Artificial Sequence Synthetic construct
HGEGTATSDLSKQMEEEAVRLFIEWLKNGGPSSGAPPPS

SEQ ID NO: 16 - protein, 39 aa, Artificial Sequence
  Description of Artificial Sequence Synthetic construct
HGEGTFSSDLSKQMEEEAVRLFIEWLKNGGPSSGAPPPS

SEQ ID NO: 17 - protein, 39 aa, Artificial Sequence
  Description of Artificial Sequence Synthetic construct
HGEGTFSTDLSKQMEEEAVRLFIEWLKNGGPSSGAPPPS

SEQ ID NO: 18 - protein, 39 aa, Artificial Sequence
  Description of Artificial Sequence Synthetic construct
HGEGTFTTDLSKQMEEEAVRLFIEWLKNGGPSSGAPPPS

SEQ ID NO: 19 - protein, 39 aa, Artificial Sequence
  Description of Artificial Sequence Synthetic construct
HGEGTFTSELSKQMEEEAVRLFIEWLKNGGPSSGAPPPS

SEQ ID NO: 20 - protein, 39 aa, Artificial Sequence
  Description of Artificial Sequence Synthetic construct
HGEGTFTSDGSKQMEEEAVRLFIEWLKNGGPSSGAPPPS

SEQ ID NO: 21 - protein, 39 aa, Artificial Sequence
  Description of Artificial Sequence Synthetic construct
HGEGTFTSDGSKQLEEEAVRLFIEFLKNGGPSSGAPPPS

SEQ ID NO: 22 - protein, 39 aa, Artificial Sequence
  Description of Artificial Sequence Synthetic construct
HGEGTFTSDLSKQGEEEAVRLFIEWLKNGGPSSGAPPPS

SEQ ID NO: 23 - protein, 39 aa, Artificial Sequence
  Description of Artificial Sequence Synthetic construct
HGEGTFTSDLSKQGEEEAVRLFIEFLKNGGPSSGAPPPS

SEQ ID NO: 24 - protein, 39 aa, Artificial Sequence
  Description of Artificial Sequence Synthetic construct
HGEGTFTSDLSKQMEEEAVRLAIEWLKNGGPSSGAPPPS

SEQ ID NO: 25 - protein, 39 aa, Artificial Sequence
  Description of Artificial Sequence Synthetic construct
HGEGTFTSDLSKQMEEEAVRLFVEWLKNGGPSSGAPPPS

SEQ ID NO: 26 - protein, 39 aa, Artificial Sequence
  Description of Artificial Sequence Synthetic construct
HGEGTFTSDLSKQLEEEAVRLFVEFLKNGGPSSGAPPPS

SEQ ID NO: 27 - protein, 39 aa, Artificial Sequence
  Description of Artificial Sequence Synthetic construct
HGEGTFTSDLSKQMEEEAVRLFGEWLKNGGPSSGAPPPS

SEQ ID NO: 28 - protein, 39 aa, Artificial Sequence
  Description of Artificial Sequence Synthetic construct
HGEGTFTSDLSKQLEEEAVRLFGEFLKNGGPSSGAPPPS

SEQ ID NO: 29 - protein, 39 aa, Artificial Sequence
  Description of Artificial Sequence Synthetic construct
HGEGTFTSDLSKQMEEEAVRLFIDWLKNGGPSSGAPPPS

SEQ ID NO: 30 - protein, 39 aa, Artificial Sequence
  Description of Artificial Sequence Synthetic construct
HAEGTFTSDLSKQLEEEAVRLFIEFLKNGGPSSGAPPPS

SEQ ID NO: 31 - protein, 39 aa, Artificial Sequence; identical to SEQ ID NO: 2
  Description of Artificial Sequence Synthetic construct

SEQ ID NO: 32 - protein, 39 aa, Artificial Sequence; identical to SEQ ID NO: 2
  Description of Artificial Sequence Synthetic construct

SEQ ID NO: 33 - protein, 39 aa, Artificial Sequence; identical to SEQ ID NO: 2
  Description of Artificial Sequence Synthetic construct

SEQ ID NO: 34 - protein, 39 aa, Artificial Sequence; identical to SEQ ID NO: 2
  Description of Artificial Sequence Synthetic construct

SEQ ID NO: 35 - protein, 39 aa, Artificial Sequence; identical to SEQ ID NO: 9
  Description of Artificial Sequence Synthetic construct

SEQ ID NO: 36 - protein, 39 aa, Artificial Sequence; identical to SEQ ID NO: 9
  Description of Artificial Sequence Synthetic construct

SEQ ID NO: 37 - protein, 39 aa, Artificial Sequence
  Description of Artificial Sequence Synthetic construct
HGEGTFTSDLSKQMEEEAVRLFIEWLKNGGASSGAAAAS

SEQ ID NO: 38 - protein, 39 aa, Artificial Sequence
  Description of Artificial Sequence Synthetic construct
HGEGTFTSDLSKQMEEEAVRLFIEWLKNGGPSSGAAAAS

SEQ ID NO: 39 - protein, 39 aa, Artificial Sequence
  Description of Artificial Sequence Synthetic construct
HGEGTFTSDLSKQLEEEAVRLFIEFLKNGGASSGAAAAS

SEQ ID NO: 40 - protein, 28 aa, Artificial Sequence
  Description of Artificial Sequence Synthetic construct
HGEGTFTSDLSKQMEEEAVRLFIEWLKN

SEQ ID NO: 41 - protein, 28 aa, Artificial Sequence
  Description of Artificial Sequence Synthetic construct
HGEGTFTSDLSKQLEEEAVRLFIEFLKN

SEQ ID NO: 42 - protein, 28 aa, Artificial Sequence
  Description of Artificial Sequence Synthetic construct
HAEGTFTSDLSKQLEEEAVRLFIEFLKN

SEQ ID NO: 43 - protein, 28 aa, Artificial Sequence
  Description of Artificial Sequence Synthetic construct
HGEGAFTSDLSKQLEEEAVRLFIEFLKN

SEQ ID NO: 44 - protein, 28 aa, Artificial Sequence
  Description of Artificial Sequence Synthetic construct
HGEGTATSDLSKQLEEEAVRLFIEFLKN

SEQ ID NO: 45 - protein, 28 aa, Artificial Sequence
  Description of Artificial Sequence Synthetic construct
HGEGTFTADLSKQLEEEAVRLFIEFLKN

SEQ ID NO: 46 - protein, 28 aa, Artificial Sequence
  Description of Artificial Sequence Synthetic construct
HGEGTFTSDASKQLEEEAVRLFIEFLKN

SEQ ID NO: 47 - protein, 28 aa, Artificial Sequence
  Description of Artificial Sequence Synthetic construct
HGEGTFTSDLAKQLEEEAVRLFIEFLKN

SEQ ID NO: 48 - protein, 28 aa, Artificial Sequence
  Description of Artificial Sequence Synthetic construct
HGEGTFTSDLSAQLEEEAVRLFIEFLKN

SEQ ID NO: 49 - protein, 28 aa, Artificial Sequence
  Description of Artificial Sequence Synthetic construct
HGEGTFTSDLSKALEEEAVRLFIEFLKN

SEQ ID NO: 50 - protein, 28 aa, Artificial Sequence
  Description of Artificial Sequence Synthetic construct
HGEGTFTSDLSKQAEEEAVRLFIEFLKN

SEQ ID NO: 51 - protein, 28 aa, Artificial Sequence
  Description of Artificial Sequence Synthetic construct
HGEGTFTSDLSKQLAEEAVRLFIEFLKN

SEQ ID NO: 52 - protein, 28 aa, Artificial Sequence
  Description of Artificial Sequence Synthetic construct
HGEGTFTSDLSKQLEAEAVRLFIEFLKN

SEQ ID NO: 53 - protein, 28 aa, Artificial Sequence
  Description of Artificial Sequence Synthetic construct
HGEGTFTSDLSKQLEEAAVRLFIEFLKN

SEQ ID NO: 54 - protein, 28 aa, Artificial Sequence
  Description of Artificial Sequence Synthetic construct
HGEGTFTSDLSKQLEEEAARLFIEFLKN

SEQ ID NO: 55 - protein, 28 aa, Artificial Sequence
  Description of Artificial Sequence Synthetic construct
HGEGTFTSDLSKQLEEEAVALFIEFLKN

SEQ ID NO: 56 - protein, 28 aa, Artificial Sequence
  Description of Artificial Sequence Synthetic construct
HGEGTFTSDLSKQLEEEAVRAFIEFLKN

SEQ ID NO: 57 - protein, 28 aa, Artificial Sequence
  Description of Artificial Sequence Synthetic construct
HGEGTFTSDLSKQLEEEAVRLFIAFLKN

SEQ ID NO: 58 - protein, 28 aa, Artificial Sequence
  Description of Artificial Sequence Synthetic construct
HGEGTFTSDLSKQLEEEAVRLFIEALKN

SEQ ID NO: 59 - protein, 28 aa, Artificial Sequence
  Description of Artificial Sequence Synthetic construct
HGEGTFTSDLSKQLEEEAVRLFIEFAKN

SEQ ID NO: 60 - protein, 28 aa, Artificial Sequence
  Description of Artificial Sequence Synthetic construct
HGEGTFTSDLSKQLEEEAVRLFIEFLAN

SEQ ID NO: 61 - protein, 28 aa, Artificial Sequence
  Description of Artificial Sequence Synthetic construct
HGEGTFTSDLSKQLEEEAVRLFIEFLKA

SEQ ID NO: 62 - protein, 38 aa, Artificial Sequence
  Description of Artificial Sequence Synthetic construct
HGEGTFTSDLSKQMEEEAVRLFIEWLKNGGPSSGAPPP

SEQ ID NO: 63 - protein, 38 aa, Artificial Sequence
  Description of Artificial Sequence Synthetic construct
HGEGTFTSDLSKQLEEEAVRLFIEFLKNGGPSSGAPPP

SEQ ID NO: 64 - protein, 37 aa, Artificial Sequence
  Description of Artificial Sequence Synthetic construct
HGEGTFTSDLSKQMEEEAVRLFIEWLKNGGPSSGAPP

SEQ ID NO: 65 - protein, 37 aa, Artificial Sequence
  Description of Artificial Sequence Synthetic construct
HGEGTFTSDLSKQLEEEAVRLFIEFLKNGGPSSGAPP

SEQ ID NO: 66 - protein, 36 aa, Artificial Sequence
  Description of Artificial Sequence Synthetic construct
HGEGTFTSDLSKQMEEEAVRLFIEWLKNGGPSSGAP

SEQ ID NO: 67 - protein, 36 aa, Artificial Sequence
  Description of Artificial Sequence Synthetic construct
HGEGTFTSDLSKQLEEEAVRLFIEFLKNGGPSSGAP

SEQ ID NO: 68 - protein, 35 aa, Artificial Sequence
  Description of Artificial Sequence Synthetic construct
HGEGTFTSDLSKQMEEEAVRLFIEWLKNGGPSSGA

SEQ ID NO: 69 - protein, 35 aa, Artificial Sequence
  Description of Artificial Sequence Synthetic construct
HGEGTFTSDLSKQLEEEAVRLFIEFLKNGGPSSGA

SEQ ID NO: 70 - protein, 34 aa, Artificial Sequence
  Description of Artificial Sequence Synthetic construct
HGEGTFTSDLSKQMEEEAVRLFIEWLKNGGPSSG

SEQ ID NO: 71 - protein, 34 aa, Artificial Sequence
  Description of Artificial Sequence Synthetic construct
HGEGTFTSDLSKQLEEEAVRLFIEFLKNGGPSSG

SEQ ID NO: 72 - protein, 33 aa, Artificial Sequence
  Description of Artificial Sequence Synthetic construct
HGEGTFTSDLSKQMEEEAVRLFIEWLKNGGPSS

SEQ ID NO: 73 - protein, 33 aa, Artificial Sequence
  Description of Artificial Sequence Synthetic construct
HGEGTFTSDLSKQLEEEAVRLFIEFLKNGGPSS

SEQ ID NO: 74 - protein, 32 aa, Artificial Sequence
  Description of Artificial Sequence Synthetic construct
HGEGTFTSDLSKQMEEEAVRLFIEWLKNGGPS

SEQ ID NO: 75 - protein, 32 aa, Artificial Sequence
  Description of Artificial Sequence Synthetic construct
HGEGTFTSDLSKQLEEEAVRLFIEFLKNGGPS

SEQ ID NO: 76 - protein, 31 aa, Artificial Sequence
  Description of Artificial Sequence Synthetic construct
HGEGTFTSDLSKQMEEEAVRLFIEWLKNGGP

SEQ ID NO: 77 - protein, 31 aa, Artificial Sequence
  Description of Artificial Sequence Synthetic construct
HGEGTFTSDLSKQLEEEAVRLFIEFLKNGGP

SEQ ID NO: 78 - protein, 30 aa, Artificial Sequence
  Description of Artificial Sequence Synthetic construct
HGEGTFTSDLSKQLEEEAVRLFIEFLKNGG

SEQ ID NO: 79 - protein, 29 aa, Artificial Sequence
  Description of Artificial Sequence Synthetic construct
HGEGTFTSDLSKQMEEEAVRLFIEWLKNG

SEQ ID NO: 80 - protein, 29 aa, Artificial Sequence
  Description of Artificial Sequence Synthetic construct
HGEGTFTSDLSKQLEEEAVRLFIEFLKNG

SEQ ID NO: 81 - protein, 38 aa, Artificial Sequence; identical to SEQ ID NO: 62
  Description of Artificial Sequence Synthetic construct

SEQ ID NO: 82 - protein, 38 aa, Artificial Sequence; identical to SEQ ID NO: 62
  Description of Artificial Sequence Synthetic construct

SEQ ID NO: 83 - protein, 37 aa, Artificial Sequence
  Description of Artificial Sequence Synthetic construct
HGEGTFTSDLSKQMEEEAVRLFIEWLKNGGASSGAPP

SEQ ID NO: 84 - protein, 37 aa, Artificial Sequence
  Description of Artificial Sequence Synthetic construct
HGEGTFTSDLSKQMEEEAVRLFIEWLKNGGASSGAAA

SEQ ID NO: 85 - protein, 37 aa, Artificial Sequence; identical to SEQ ID NO: 64
  Description of Artificial Sequence Synthetic construct

SEQ ID NO: 86 - protein, 36 aa, Artificial Sequence; identical to SEQ ID NO: 66
  Description of Artificial Sequence Synthetic construct

SEQ ID NO: 87 - protein, 35 aa, Artificial Sequence
  Description of Artificial Sequence Synthetic construct
RGEGTFTSDLSKQMEEEAVRLFIEWLKNGGPSSGA

SEQ ID NO: 88 - protein, 30 aa, Artificial Sequence
  Description of Artificial Sequence Synthetic construct
HGDGTFTSDLSKQMEEEAVRLFIEWLKNGG

SEQ ID NO: 89 - protein, 28 aa, Artificial Sequence
  Description of Artificial Sequence Synthetic construct
HGEGTATSDLSKQLEEEAVRLFIEFLKN

SEQ ID NO: 90 - protein, 28 aa, Artificial Sequence
  Description of Artificial Sequence Synthetic construct
HGEGTFSSDLSKQMEEEAVRLFIEWLKN

SEQ ID NO: 91 - protein, 28 aa, Artificial Sequence
  Description of Artificial Sequence Synthetic construct
HGEGTFSTDLSKQMEEEAVRLFIEWLKN

SEQ ID NO: 92 - protein, 28 aa, Artificial Sequence
  Description of Artificial Sequence Synthetic construct
HGEGTFTSELSKQMAEEAVRLFIEWLKN

SEQ ID NO: 93 - protein, 28 aa, Artificial Sequence
  Description of Artificial Sequence Synthetic construct
HGEGTFTSDGSKQLEEEAVRLFIEFLKN

SEQ ID NO: 94 - protein, 28 aa, Artificial Sequence
  Description of Artificial Sequence Synthetic construct
HGEGTFTSDLSKQLEEEAVRLAIEFLKN

SEQ ID NO: 95 - protein, 28 aa, Artificial Sequence
  Description of Artificial Sequence Synthetic construct
HGEGTFTSDLSKQMEEEAVRLFGEWLKN

SEQ ID NO: 96 - protein, 28 aa, Artificial Sequence
  Description of Artificial Sequence Synthetic construct
HGEGTFTSDLSKQLEEEAVRLFIDFLKN

SEQ ID NO: 97 - protein, 33 aa, Artificial Sequence
  Description of Artificial Sequence Synthetic construct
HGEGTFTSDASKQLEEEAVRLFIEFLKNGGPSS

SEQ ID NO: 98 - protein, 29 aa, Artificial Sequence
  Description of Artificial Sequence Synthetic construct
HGEGTFTSDASKQMEEEAVRLFIEWLKNG

SEQ ID NO: 99 - protein, 37 aa, Artificial Sequence
  Description of Artificial Sequence Synthetic construct
HGEGTFTSDASKQMEEEAVRLFIEWLKNGGPSSGAPP

SEQ ID NO: 100 - protein, 28 aa, Artificial Sequence
  Description of Artificial Sequence Synthetic construct
AGEGTFTSDLSKQLEEEAVRLFIEFLKN

SEQ ID NO: 101 - protein, 28 aa, Artificial Sequence
  Description of Artificial Sequence Synthetic construct
HGAGTFTSDLSKQLEEEAVRLFIEFLKN

SEQ ID NO: 102 - protein, 28 aa, Artificial Sequence
  Description of Artificial Sequence Synthetic construct
HGEATFTSDLSKQLEEEAVRLFIEFLKN

SEQ ID NO: 103 - protein, 28 aa, Artificial Sequence
  Description of Artificial Sequence Synthetic construct
HGEGTFTSALSKQLEEEAVRLFIEFLKN

SEQ ID NO: 104 - protein, 28 aa, Artificial Sequence
  Description of Artificial Sequence Synthetic construct
AGEGTFTSDLSKQMEEEAVRLFIEWLKN

SEQ ID NO: 105 - protein, 28 aa, Artificial Sequence
  Description of Artificial Sequence Synthetic construct
HGAGTFTSDLSKQMEEEAVRLFIEWLKN

SEQ ID NO: 106 - protein, 28 aa, Artificial Sequence
  Description of Artificial Sequence Synthetic construct
HGEATFTSDLSKQMEEEAVRLFIEWLKN

SEQ ID NO: 107 - protein, 28 aa, Artificial Sequence
  Description of Artificial Sequence Synthetic construct
HGEGTFTSALSKQMEEEAVRLFIEWLKN

SEQ ID NO: 108 - protein, 28 aa, Artificial Sequence
  Description of Artificial Sequence Synthetic construct
HGEGTFTSDASKQMEEEAVRLFIEWLKN

SEQ ID NO: 109 - protein, 28 aa, Artificial Sequence
  Description of Artificial Sequence Synthetic construct
AAEGTFTSDLSKQMEEEAVRLFIEWLKN

SEQ ID NO: 110 - protein, 28 aa, Artificial Sequence
  Description of Artificial Sequence Synthetic construct
AAEGTFTSDLSKQLEEEAVRLFIEFLKN

SEQ ID NO: 111 - protein, 28 aa, Artificial Sequence
  Description of Artificial Sequence Synthetic construct
AGDGTFTSDLSKQMEEEAVRLFIEWLKN

SEQ ID NO: 112 - protein, 28 aa, Artificial Sequence
  Description of Artificial Sequence Synthetic construct
AGDGTFTSDLSKQLEEEAVRLFIEFLKN

SEQ ID NO: 113 - protein, 28 aa, Artificial Sequence
  Description of Artificial Sequence Synthetic construct
AGDGAFTSDLSKQMEEEAVRLFIEWLKN

SEQ ID NO: 114 - protein, 28 aa, Artificial Sequence
  Description of Artificial Sequence Synthetic construct
AGDGAFTSDLSKQLEEEAVRLFIEFLKN

SEQ ID NO: 115 - protein, 28 aa, Artificial Sequence
  Description of Artificial Sequence Synthetic construct
AGDGTATSDLSKQMEEEAVRLFIEWLKN

SEQ ID NO: 116 - protein, 28 aa, Artificial Sequence
  Description of Artificial Sequence Synthetic construct
AGDGTATSDLSKQLEEEAVRLFIEFLKN

SEQ ID NO: 117 - protein, 28 aa, Artificial Sequence
  Description of Artificial Sequence Synthetic construct
AGDGTFSSDLSKQMEEEAVRLFIEWLKN

SEQ ID NO: 118 - protein, 28 aa, Artificial Sequence
  Description of Artificial Sequence Synthetic construct
AGDGTFSSDLSKQLEEEAVRLFIEFLKN

SEQ ID NO: 119 - protein, 28 aa, Artificial Sequence
  Description of Artificial Sequence Synthetic construct
AGDGTFTADLSKQMEEEAVRLFIEWLKN

SEQ ID NO: 120 - protein, 28 aa, Artificial Sequence
  Description of Artificial Sequence Synthetic construct
AGDGTFTADLSKQLEEEAVRLFIEFLKN

SEQ ID NO: 121 - protein, 28 aa, Artificial Sequence
  Description of Artificial Sequence Synthetic construct
AGDGTFTSALSKQMEEEAVRLFIEWLKN

SEQ ID NO: 122 - protein, 28 aa, Artificial Sequence
  Description of Artificial Sequence Synthetic construct
AGDGTFTSALSKQLEEEAVRLFIEFLKN

SEQ ID NO: 123 - protein, 28 aa, Artificial Sequence
  Description of Artificial Sequence Synthetic construct
AGDGTFTSELSKQMEEEAVRLFIEWLKN

SEQ ID NO: 124 - protein, 28 aa, Artificial Sequence
  Description of Artificial Sequence Synthetic construct
AGDGTFTSELSKQLEEEAVRLFIEFLKN

SEQ ID NO: 125 - protein, 28 aa, Artificial Sequence
  Description of Artificial Sequence Synthetic construct
AGDGTFTSDASKQMEEEAVRLFIEWLKN

SEQ ID NO: 126 - protein, 28 aa, Artificial Sequence
  Description of Artificial Sequence Synthetic construct
AGDGTFTSDASKQLEEEAVRLFIEFLKN

SEQ ID NO: 127 - protein, 28 aa, Artificial Sequence
  Description of Artificial Sequence Synthetic construct
AGDGTFTSDGSKQMEEEAVRLFIEWLKN

SEQ ID NO: 128 - protein, 28 aa, Artificial Sequence
  Description of Artificial Sequence Synthetic construct
AGDGTFTSDGSKQLEEEAVRLFIEFLKN

SEQ ID NO: 129 - protein, 28 aa, Artificial Sequence
  Description of Artificial Sequence Synthetic construct
AGDGTFTSDLAKQMEEEAVRLFIEWLKN

SEQ ID NO: 130 - protein, 28 aa, Artificial Sequence
  Description of Artificial Sequence Synthetic construct
AGDGTFTSDLAKQLEEEAVRLFIEFLKN

SEQ ID NO: 131 - protein, 28 aa, Artificial Sequence
  Description of Artificial Sequence Synthetic construct
AGDGTFTSDLSAQMEEEAVRLFIEWLKN

SEQ ID NO: 132 - protein, 28 aa, Artificial Sequence
  Description of Artificial Sequence Synthetic construct
AGDGTFTSDLSAQLEEEAVRLFIEFLKN

SEQ ID NO: 133 - protein, 28 aa, Artificial Sequence
  Description of Artificial Sequence Synthetic construct
AGDGTFTSDLSKAMEEEAVRLFIEWLKN

SEQ ID NO: 134 - protein, 28 aa, Artificial Sequence
  Description of Artificial Sequence Synthetic construct
AGDGTFTSDLSKALEEEAVRLFIEFLKN

SEQ ID NO: 135 - protein, 28 aa, Artificial Sequence
  Description of Artificial Sequence Synthetic construct
AGDGTFTSDLSKQAEEEAVRLFIEWLKN

SEQ ID NO: 136 - protein, 28 aa, Artificial Sequence
  Description of Artificial Sequence Synthetic construct
AGDGTFTSDLSKQAEEEAVRLFIEFLKN

SEQ ID NO: 137 - protein, 28 aa, Artificial Sequence
  Description of Artificial Sequence Synthetic construct
AGDGTFTSDLSKQGEEEAVRLFIEWLKN

SEQ ID NO: 138 - protein, 28 aa, Artificial Sequence
  Description of Artificial Sequence Synthetic construct
AGDGTFTSDLSKQGEEEAVRLFIEFLKN

SEQ ID NO: 139 - protein, 28 aa, Artificial Sequence
  Description of Artificial Sequence Synthetic construct
AGDGTFTSDLSKQMAEEAVRLFIEWLKN

SEQ ID NO: 140 - protein, 28 aa, Artificial Sequence
  Description of Artificial Sequence Synthetic construct
AGDGTFTSDLSKQLAEEAVRLFIEFLKN

SEQ ID NO: 141 - protein, 28 aa, Artificial Sequence
  Description of Artificial Sequence Synthetic construct
AGDGTFTSDLSKQMEAEAVRLFIEWLKN

SEQ ID NO: 142 - protein, 28 aa, Artificial Sequence
  Description of Artificial Sequence Synthetic construct
AGDGTFTSDLSKQLEAEAVRLFIEFLKN

SEQ ID NO: 143 - protein, 28 aa, Artificial Sequence
  Description of Artificial Sequence Synthetic construct
AGDGTFTSDLSKQMEEAAVRLFIEWLKN

SEQ ID NO: 144 - protein, 28 aa, Artificial Sequence
  Description of Artificial Sequence Synthetic construct
AGDGTFTSDLSKQLEEAAVRLFIEFLKN

SEQ ID NO: 145 - protein, 28 aa, Artificial Sequence
  Description of Artificial Sequence Synthetic construct
AGDGTFTSDLSKQMEEEAARLFIEWLKN

SEQ ID NO: 146 - protein, 28 aa, Artificial Sequence
  Description of Artificial Sequence Synthetic construct
AGDGTFTSDLSKQLEEEAARLFIEFLKN

SEQ ID NO: 147 - protein, 28 aa, Artificial Sequence
  Description of Artificial Sequence Synthetic construct
AGDGTFTSDLSKQMEEEAVALFIEWLKN

SEQ ID NO: 148 - protein, 28 aa, Artificial Sequence
  Description of Artificial Sequence Synthetic construct
AGDGTFTSDLSKQLEEEAVALFIEFLKN

SEQ ID NO: 149 - protein, 28 aa, Artificial Sequence
  Description of Artificial Sequence Synthetic construct
AGDGTFTSDLSKQMEEEAVRAFIEWLKN

SEQ ID NO: 150 - protein, 28 aa, Artificial Sequence
  Description of Artificial Sequence Synthetic construct
AGDGTFTSDLSKQLEEEAVRAFIEFLKN

SEQ ID NO: 151 - protein, 28 aa, Artificial Sequence
  Description of Artificial Sequence Synthetic construct
AGDGTFTSDLSKQMEEEAVRLAIEWLKN

SEQ ID NO: 152 - protein, 28 aa, Artificial Sequence
  Description of Artificial Sequence Synthetic construct
AGDGTFTSDLSKQLEEEAVRLAIEFLKN

SEQ ID NO: 153 - protein, 28 aa, Artificial Sequence
  Description of Artificial Sequence Synthetic construct
AGDGTFTSDLSKQMEEEAVRLFVEWLKN

SEQ ID NO: 154 - protein, 28 aa, Artificial Sequence
  Description of Artificial Sequence Synthetic construct
AGDGTFTSDLSKQLEEEAVRLFVEFLKN

SEQ ID NO: 155 - protein, 28 aa, Artificial Sequence
  Description of Artificial Sequence Synthetic construct
AGDGTFTSDLSKQMEEEAVRLFGEWLKN

SEQ ID NO: 156 - protein, 28 aa, Artificial Sequence
  Description of Artificial Sequence Synthetic construct
AGDGTFTSDLSKQLEEEAVRLFGEFLKN

SEQ ID NO: 157 - protein, 28 aa, Artificial Sequence
  Description of Artificial Sequence Synthetic construct
AGDGTFTSDLSKQMEEEAVRLFIDWLKN

SEQ ID NO: 158 - protein, 28 aa, Artificial Sequence
  Description of Artificial Sequence Synthetic construct
AGDGTFTSDLSKQLEEEAVRLFIDFLKN

SEQ ID NO: 159 - protein, 28 aa, Artificial Sequence
  Description of Artificial Sequence Synthetic construct
AGDGTFTSDLSKQMEEEAVRLFIEALKN

SEQ ID NO: 160 - protein, 28 aa, Artificial Sequence
  Description of Artificial Sequence Synthetic construct
AGDGTFTSDLSKQLEEEAVRLFIEALKN

SEQ ID NO: 161 - protein, 28 aa, Artificial Sequence
  Description of Artificial Sequence Synthetic construct
AGDGTFTSDLSKQMEEEAVRLFIEWAKN

SEQ ID NO: 162 - protein, 28 aa, Artificial Sequence
  Description of Artificial Sequence Synthetic construct
AGDGTFTSDLSKQLEEEAVRLFIEFAKN

SEQ ID NO: 163 - protein, 28 aa, Artificial Sequence
  Description of Artificial Sequence Synthetic construct
AGDGTFTSDLSKQMEEEAVRLFIEWLAN

SEQ ID NO: 164 - protein, 28 aa, Artificial Sequence
  Description of Artificial Sequence Synthetic construct
AGDGTFTSDLSKQLEEEAVRLFIEFLAN

SEQ ID NO: 165 - protein, 28 aa, Artificial Sequence
  Description of Artificial Sequence Synthetic construct
AGDGTFTSDLSKQMEEEAVRLFIEWLKA

SEQ ID NO: 166 - protein, 28 aa, Artificial Sequence
  Description of Artificial Sequence Synthetic construct
AGDGTFTSDLSKQLEEEAVRLFIEFLKA

SEQ ID NO: 167 - protein, 38 aa, Artificial Sequence
  Description of Artificial Sequence Synthetic construct
AGEGTFTSDLSKQMEEEAVRLFIEWLKNGGPSSGAPPP

SEQ ID NO: 168 - protein, 38 aa, Artificial Sequence
  Description of Artificial Sequence Synthetic construct
HGAGTFTSDLSKQLEEEAVRLFIEFLKNGGPSSGAPPP

SEQ ID NO: 169 - protein, 37 aa, Artificial Sequence
  Description of Artificial Sequence Synthetic construct
HGEATFTSDLSKQMEEEAVRLFIEWLKNGGPSSGAPP

SEQ ID NO: 170 - protein, 36 aa, Artificial Sequence
  Description of Artificial Sequence Synthetic construct
HGEGTFTSALSKQMEEEAVRLFIEWLKNGGPSSGAP

SEQ ID NO: 171 - protein, 36 aa, Artificial Sequence
  Description of Artificial Sequence Synthetic construct
AGEGTFTSDASKQLEEEAVRLFIEFLKNGGPSSGAP

SEQ ID NO: 172 - protein, 35 aa, Artificial Sequence
  Description of Artificial Sequence Synthetic construct
AGEGTFTSDLSKQMEEEAVRLFIEWLKNGGPSSGA

SEQ ID NO: 173 - protein, 35 aa, Artificial Sequence
  Description of Artificial Sequence Synthetic construct
HGAGTFTSDLSKQLEEEAVRLFIEFLKNGGPSSGA

SEQ ID NO: 174 - protein, 34 aa, Artificial Sequence
  Description of Artificial Sequence Synthetic construct
HGEATFTSDLSKQMEEEAVRLFIEWLKNGGPSSG

SEQ ID NO: 175 - protein, 33 aa, Artificial Sequence
  Description of Artificial Sequence Synthetic construct
HGEGTFTSALSKQMEEEAVRLFIEWLKNGGPSS

SEQ ID NO: 176 - protein, 32 aa, Artificial Sequence
  Description of Artificial Sequence Synthetic construct
AGEGTFTSDLSKQMEEEAVRLFIEWLKNGGPS

SEQ ID NO: 177 - protein, 32 aa, Artificial Sequence
  Description of Artificial Sequence Synthetic construct
HGAGTFTSDLSKQLEEEAVRLFIEFLKNGGPS

SEQ ID NO: 178 - protein, 31 aa, Artificial Sequence
  Description of Artificial Sequence Synthetic construct
HGEATFTSDLSKQMEEEAVRLFIEWLKNGGP

SEQ ID NO: 179 - protein, 30 aa, Artificial Sequence
  Description of Artificial Sequence Synthetic construct
HGEGTFTSALSKQLEEEAVRLFIEFLKNGG

SEQ ID NO: 180 - protein, 29 aa, Artificial Sequence
  Description of Artificial Sequence Synthetic construct
AGEGTFTSDLSKQLEEEAVRLFIEFLKNG

SEQ ID NO: 181 - protein, 38 aa, Artificial Sequence
  Description of Artificial Sequence Synthetic construct
HGAGTFTSDLSKQMEEEAVRLFIEWLKNGGPSSGAPPP

SEQ ID NO: 182 - protein, 38 aa, Artificial Sequence
  Description of Artificial Sequence Synthetic construct
HGEATFTSDLSKQMEEEAVRLFIEWLKNGGPSSGAPPP

SEQ ID NO: 183 - protein, 37 aa, Artificial Sequence
  Description of Artificial Sequence Synthetic construct
HGEGTFTSALSKQMEEEAVRLFIEWLKNGGASSGAAA

SEQ ID NO: 184 - protein, 36 aa, Artificial Sequence
  Description of Artificial Sequence Synthetic construct
AGEGTFTSDLSKQMEEEAVRLFIEWLKNGGPSSGAP

SEQ ID NO: 185 - protein, 35 aa, Artificial Sequence
  Description of Artificial Sequence Synthetic construct
HGAGTFTSDLSKQMEEEAVRLFIEWLKNGGPSSGA

SEQ ID NO: 186 - protein, 30 aa, Artificial Sequence
  Description of Artificial Sequence Synthetic construct
HGDATFTSDLSKQMEEEAVRLFIEWLKNGG

SEQ ID NO: 187 - protein, 39 aa, Artificial Sequence
  Description of Artificial Sequence Synthetic construct
AGEGTFTSDLSKQMEEEAVRLFIEWLKNGGPSSGAPPPS

SEQ ID NO: 188 - protein, 39 aa, Artificial Sequence
  Description of Artificial Sequence Synthetic construct
AGAGTFTSDLSKQLEEEAVRLFIEFLKNGGPSSGAPPPS

SEQ ID NO: 189 - protein, 36 aa, Artificial Sequence
  Description of Artificial Sequence Synthetic construct
APLEPVYPGDNATPEQMAQYAADLRRYINMLTRPRY

SEQ ID NO: 190 - protein, 36 aa, Homo sapien
YPIKPEAPGEDASPEELNRYYASLRHYLNLVTRQRY

SEQ ID NO: 191 - protein, 34 aa, Artificial Sequence
  Description of Artificial Sequence Synthetic construct
IKPEAPGEDASPEELNRYYASLRHYLNLVTRQRY

SEQ ID NO: 192 - protein, 36 aa, Artificial Sequence
  Description of Artificial Sequence Synthetic construct
YPSKPDNPGEDAPAEDMARYYSALRHYINLITRQRY

SEQ ID NO: 193 - protein, 34 aa, Artificial Sequence
  Description of Artificial Sequence Synthetic construct
SKPDNPGEDAPAEDMARYYSALRHYINLITRQRY

SEQ ID NO: 194 - protein, 15 aa, Artificial Sequence
  Description of Artificial Sequence Synthetic construct
ASLRHYLNLVTRQRY

SEQ ID NO: 195 - protein, 31 aa, Artificial Sequence
  Description of Artificial Sequence Synthetic construct
HVEGTFTSDVSSYLEEQAAKEFIAWLVKGRG

SEQ ID NO: 196 - protein, 31 aa, Artificial Sequence
  Description of Artificial Sequence Synthetic construct
HVEGTFTSDVSSYLEDQAAKEFIAWLVKGRG

SEQ ID NO: 197 - protein, 31 aa, Artificial Sequence
  Description of Artificial Sequence Synthetic construct
HVEGTFTSDVSSYLERQAAKEFIAWLVKGRG

SEQ ID NO: 198 - protein, 31 aa, Artificial Sequence
  Description of Artificial Sequence Synthetic construct
HVEGTFTSDVSSYLEKQAAKEFIAWLVKGRG

SEQ ID NO: 199 - protein, 31 aa, Artificial Sequence
  Description of Artificial Sequence Synthetic construct
HGEGTFTSDVSSYLEEQAAKEFIAWLVKGRG

SEQ ID NO: 200 - protein, 31 aa, Artificial Sequence
  Description of Artificial Sequence Synthetic construct
HGEGTFTSDVSSYLEDQAAKEFIAWLVKGRG

SEQ ID NO: 201 - protein, 31 aa, Artificial Sequence
  Description of Artificial Sequence Synthetic construct
HGEGTFTSDVSSYLERQAAKEFIAWLVKGRG

SEQ ID NO: 202 - protein, 31 aa, Artificial Sequence
  Description of Artificial Sequence Synthetic construct
HGEGTFTSDVSSYLEKQAAKEFIAWLVKGRG

SEQ ID NO: 203 - protein, 31 aa, Artificial Sequence
  Description of Artificial Sequence Synthetic construct
HVEGTFTSDVSSYLEGQAAKEFIEWLVKGRG

SEQ ID NO: 204 - protein, 31 aa, Artificial Sequence
  Description of Artificial Sequence Synthetic construct
HGEGTFTSDVSSYLEGQAAKEFIEWLVKGRG

SEQ ID NO: 205 - protein, 31 aa, Artificial Sequence
  Description of Artificial Sequence Synthetic construct
HVEGTFTSDVSSYLEGQAAKEFIAWLVKGRH

SEQ ID NO: 206 - protein, 31 aa, Artificial Sequence
  Description of Artificial Sequence Synthetic construct
HGEGTFTSDVSSYLEGQAAKEFIAWLVKGRH

SEQ ID NO: 207 - protein, 31 aa, Artificial Sequence
  Description of Artificial Sequence Synthetic construct
HVEGTFTSDVSSYLEEQAAKAFIAWLVKGRH

SEQ ID NO: 208 - protein, 31 aa, Artificial Sequence
  Description of Artificial Sequence Synthetic construct
HVEGTFTSDVSSYLEKEAAKEFIAWLVKGRH

SEQ ID NO: 209 - protein, 31 aa, Artificial Sequence
  Description of Artificial Sequence Synthetic construct
HAEGTFTSDVSSYLEGQAAKEFIAWLVKGRG

SEQ ID NO: 210 - protein, 31 aa, Artificial Sequence
  Description of Artificial Sequence Synthetic construct
HAEGTFTSDVSSYLEEQAAKEFIAWLVKGRG

SEQ ID NO: 211 - protein, 31 aa, Artificial Sequence
  Description of Artificial Sequence Synthetic construct
HAEGTFTSDVSSYLEDQAAKEFIAWLVKGRG

SEQ ID NO: 212 - protein, 31 aa, Artificial Sequence
  Description of Artificial Sequence Synthetic construct
HAEGTFTSDVSSYLERQAAKEFIAWLVKGRG

SEQ ID NO: 213 - protein, 31 aa, Artificial Sequence
  Description of Artificial Sequence Synthetic construct
HAEGTFTSDVSSYLEKQAAKEFIAWLVKGRG

SEQ ID NO: 214 - protein, 31 aa, Artificial Sequence
  Description of Artificial Sequence Synthetic construct
HAEGTFTSDVSSYLEAQAAKEFIAWLVKGRG

SEQ ID NO: 215 - protein, 31 aa, Artificial Sequence
  Description of Artificial Sequence Synthetic construct
HVEGTFTSDVSSYLEAQAAKEFIAWLVKGRG

SEQ ID NO: 216 - protein, 31 aa, Artificial Sequence
  Description of Artificial Sequence Synthetic construct
HGEGTFTSDVSSYLEAQAAKEFIAWLVKGRG

SEQ ID NO: 217 - protein, 30 aa, Artificial Sequence
  Description of Artificial Sequence Synthetic construct
HAEGTFTSDVSSYLEEQAAKEFIAWLVKGR

SEQ ID NO: 218 - protein, 30 aa, Artificial Sequence
  Description of Artificial Sequence Synthetic construct
HAEGTFTSDVSSYLEDQAAKEFIAWLVKGR

SEQ ID NO: 219 - protein, 30 aa, Artificial Sequence
  Description of Artificial Sequence Synthetic construct
HAEGTFTSDVSSYLERQAAKEFIAWLVKGR

SEQ ID NO: 220 - protein, 30 aa, Artificial Sequence
  Description of Artificial Sequence Synthetic construct
HAEGTFTSDVSSYLEKQAAKEFIAWLVKGR

SEQ ID NO: 221 - protein, 30 aa, Artificial Sequence
  Description of Artificial Sequence Synthetic construct
HAEGTFTSDVSSYLEAQAAKEFIAWLVKGR

SEQ ID NO: 222 - protein, 30 aa, Artificial Sequence
  Description of Artificial Sequence Synthetic construct
HVEGTFTSDVSSYLEEQAAKEFIAWLVKGR

SEQ ID NO: 223 - protein, 30 aa, Artificial Sequence
  Description of Artificial Sequence Synthetic construct
HVEGTFTSDVSSYLEDQAAKEFIAWLVKGR

SEQ ID NO: 224 - protein, 30 aa, Artificial Sequence
  Description of Artificial Sequence Synthetic construct
HVEGTFTSDVSSYLERQAAKEFIAWLVKGR

SEQ ID NO: 225 - protein, 30 aa, Artificial Sequence
  Description of Artificial Sequence Synthetic construct
HVEGTFTSDVSSYLEKQAAKEFIAWLVKGR

SEQ ID NO: 226 - protein, 30 aa, Artificial Sequence
  Description of Artificial Sequence Synthetic construct
HVEGTFTSDVSSYLEAQAAKEFIAWLVKGR

SEQ ID NO: 227 - protein, 30 aa, Artificial Sequence
  Description of Artificial Sequence Synthetic construct
HGEGTFTSDVSSYLEEQAAKEFIAWLVKGR

SEQ ID NO: 228 - protein, 30 aa, Artificial Sequence
  Description of Artificial Sequence Synthetic construct
HGEGTFTSDVSSYLEDQAAKEFIAWLVKGR

SEQ ID NO: 229 - protein, 30 aa, Artificial Sequence
  Description of Artificial Sequence Synthetic construct
HGEGTFTSDVSSYLERQAAKEFIAWLVKGR

SEQ ID NO: 230 - protein, 30 aa, Artificial Sequence
  Description of Artificial Sequence Synthetic construct
HGEGTFTSDVSSYLEKQAAKEFIAWLVKGR

SEQ ID NO: 231 - protein, 30 aa, Artificial Sequence
  Description of Artificial Sequence Synthetic construct
HGEGTFTSDVSSYLEAQAAKEFIAWLVKGR

SEQ ID NO: 232 - protein, 31 aa, Artificial Sequence
  Description of Artificial Sequence Synthetic construct
HVEGTFTSDVSSYLEGKAAKEFIAWLVKGRG

SEQ ID NO: 233 - protein, 31 aa, Artificial Sequence
  Description of Artificial Sequence Synthetic construct
HVEGTFTSDVSSYLEGQAAKAFIAWLVKGRG

SEQ ID NO: 234 - protein, 31 aa, Artificial Sequence; identical to SEQ ID NO: 203
  Description of Artificial Sequence Synthetic construct

SEQ ID NO: 235 - protein, 31 aa, Artificial Sequence; identical to SEQ ID NO: 204
  Description of Artificial Sequence Synthetic construct

SEQ ID NO: 236 - protein, 31 aa, Artificial Sequence
  Description of Artificial Sequence Synthetic construct
HVEGTFTSDVSSYLEGQAAKEFIAWLVKHRG

SEQ ID NO: 237 - protein, 31 aa, Artificial Sequence; identical to SEQ ID NO: 205
  Description of Artificial Sequence Synthetic construct

SEQ ID NO: 238 - protein, 31 aa, Artificial Sequence
  Description of Artificial Sequence Synthetic construct
HVEGTFTSDVSSYLEEKAAKEFIAWLVKGRG

SEQ ID NO: 239 - protein, 31 aa, Artificial Sequence
  Description of Artificial Sequence Synthetic construct
HVEGTFTSDVSSYLEEEAAKEFIAWLVKGRG

SEQ ID NO: 240 - protein, 31 aa, Artificial Sequence
  Description of Artificial Sequence Synthetic construct
HVEGTFTSDVSSYLEEQAAKAFIAWLVKGRG

SEQ ID NO: 241 - protein, 31 aa, Artificial Sequence
  Description of Artificial Sequence Synthetic construct
HVEGTFTSDVSSYLEGQAAKEFIAWLVGKRG

SEQ ID NO: 242 - protein, 31 aa, Artificial Sequence; identical to SEQ ID NO: 205
  Description of Artificial Sequence Synthetic construct

SEQ ID NO: 243 - protein, 31 aa, Artificial Sequence; identical to SEQ ID NO: 206
  Description of Artificial Sequence Synthetic construct

SEQ ID NO: 244 - protein, 32 aa, Artificial Sequence
  Description of Artificial Sequence Synthetic construct
HXEGXXTSDXSSYLEXXXAXXFIAXLXXXXXX

SEQ ID NO: 245 - protein, 32 aa, Artificial Sequence
  Description of Artificial Sequence Synthetic construct
HXEGTXTSDXSSYLEXXAAXEFIXWLVKXRXX

SEQ ID NO: 246 - protein, 32 aa, Artificial Sequence
  Description of Artificial Sequence Synthetic construct
HXEGTFTSDVSSYLEXXAAKXFIXWLVKGRXX

SEQ ID NO: 247 - protein, 31 aa, Artificial Sequence
  Description of Artificial Sequence Synthetic construct
XXEGTFTSDVSSYLEXQAAKEFIAWLVKGRX

SEQ ID NO: 248 - protein, 37 aa, Artificial Sequence
  Description of Artificial Sequence Synthetic construct
XXNTATXATQRLXNFLXXXXXNXGXXLXXTXVGSNTY

SEQ ID NO: 249 - protein, 37 aa, Artificial Sequence [sequence omitted]

SEQ ID NO: 250 - protein, 37 aa, Artificial Sequence
  Description of Artificial Sequence Synthetic construct
KCNTATCATQRLANFLIRSSNNLGAILSPTNVGSNTY

SEQ ID NO: 251 - protein, 37 aa, Artificial Sequence
  Description of Artificial Sequence Synthetic construct
KCNTATCATQRLANFLVRTSNNLGAILSPTNVGSNTY

SEQ ID NO: 252 - protein, 37 aa, Artificial Sequence
  Description of Artificial Sequence Synthetic construct
KCNTATCATQRLANFLVRSSNNLGPVLPPTNVGSNTY

SEQ ID NO: 253 - protein, 37 aa, Artificial Sequence
  Description of Artificial Sequence Synthetic construct
KCNTATCATQRLANFLVHSNNNLGPVLSPTNVGSNTY

SEQ ID NO: 254 - protein, 37 aa, Artificial Sequence
  Description of Artificial Sequence Synthetic construct
KCNTATCATQRLTNFLVRSSHNLGAALLPTDVGSNTY

SEQ ID NO: 255 - protein, 36 aa, Artificial Sequence
  Description of Artificial Sequence Synthetic construct
CNTATCATQRLANFLVHSSNNFGAILSSTNVGSNTY

SEQ ID NO: 256 - protein, 37 aa, Artificial Sequence
  Description of Artificial Sequence Synthetic construct
KCNTATCATQRLANFLVHSSNNFGAILPSTNVGSNTY

SEQ ID NO: 257 - protein, 37 aa, Artificial Sequence
  Description of Artificial Sequence Synthetic construct
KCNTATCATQRLANFLVHSSNNFGPILPPTNVGSNTY

SEQ ID NO: 258 - protein, 37 aa, Artificial Sequence
  Description of Artificial Sequence Synthetic construct
KCNTATCATQRLANFLVRSSNNFGPILPSTNVGSNTY

SEQ ID NO: 259 - protein, 36 aa, Artificial Sequence
  Description of Artificial Sequence Synthetic construct
CNTATCATQRLANFLVHRSNNFGPILPSTNVGSNTY

SEQ ID NO: 260 - protein, 37 aa, Artificial Sequence
  Description of Artificial Sequence Synthetic construct
KCNTATCATQRLANFLVHSSNNFGPVLPPTNVGSNTY

SEQ ID NO: 261 - protein, 37 aa, Artificial Sequence
  Description of Artificial Sequence Synthetic construct
KCNTATCATQRLANFLVRSSNNFGPILPPTNVGSNTY

SEQ ID NO: 262 - protein, 36 aa, Artificial Sequence
  Description of Artificial Sequence Synthetic construct
CNTATCATQRLANFLVRSSNNFGPILPPSNVGSNTY

SEQ ID NO: 263 - protein, 36 aa, Artificial Sequence
  Description of Artificial Sequence Synthetic construct
CNTATCATQRLANFLVHSSNNFGPILPPSNVGSNTY

SEQ ID NO: 264 - protein, 37 aa, Artificial Sequence
  Description of Artificial Sequence Synthetic construct
KCNTATCATQRLANFLVHSSNNLGPVLPPTNVGSNTY

SEQ ID NO: 265 - protein, 37 aa, Artificial Sequence
  Description of Artificial Sequence Synthetic construct
KCNTATCATQRLANFLVHSSNNLGPVLPSTNVGSNTY

SEQ ID NO: 266 - protein, 36 aa, Artificial Sequence
  Description of Artificial Sequence Synthetic construct
CNTATCATQRLANFLVHSSNNLGPVLPSTNVGSNTY

SEQ ID NO: 267 - protein, 37 aa, Artificial Sequence
  Description of Artificial Sequence Synthetic construct
KCNTATCATQRLANFLVRSSNNLGPVLPSTNVGSNTY

SEQ ID NO: 268 - protein, 37 aa, Artificial Sequence
  Description of Artificial Sequence Synthetic construct
KCNTATCATQRLANFLVRSSNNLGPILPPTNVGSNTY

SEQ ID NO: 269 - protein, 37 aa, Artificial Sequence
  Description of Artificial Sequence Synthetic construct
KCNTATCATQRLANFLVRSSNNLGPILPSTNVGSNTY

SEQ ID NO: 270 - protein, 37 aa, Artificial Sequence
  Description of Artificial Sequence Synthetic construct
KCNTATCATQRLANFLIHSSNNLGPILPPTNVGSNTY

SEQ ID NO: 271 - protein, 37 aa, Artificial Sequence
  Description of Artificial Sequence Synthetic construct
KCNTATCATQRLANFLVISSNNFGPILPPTNVGSNTY

SEQ ID NO: 272 - protein, 36 aa, Artificial Sequence
  Description of Artificial Sequence Synthetic construct
CNTATCATQRLANFLIHSSNNLGPILPPTNVGSNTY

SEQ ID NO: 273 - protein, 37 aa, Artificial Sequence
  Description of Artificial Sequence Synthetic construct
KCNTATCATQRLANFLIRSSNNLGAILSSTNVGSNTY

SEQ ID NO: 274 - protein, 37 aa, Artificial Sequence
  Description of Artificial Sequence Synthetic construct
KCNTATCATQRLANFLIRSSNNLGAVLSPTNVGSNTY

SEQ ID NO: 275 - protein, 37 aa, Artificial Sequence
  Description of Artificial Sequence Synthetic construct
KCNTATCATQRLANFLIRSSNNLGPVLPPTNVGSNTY

SEQ ID NO: 276 - protein, 37 aa, Artificial Sequence
  Description of Artificial Sequence Synthetic construct
KCNTATCATQRLTNFLVHSSHNLGAALLPTDVGSNTY

SEQ ID NO: 277 - protein, 37 aa, Artificial Sequence
  Description of Artificial Sequence Synthetic construct
KCNTATCATQRLTNFLVHSSHNLGAALSPTDVGSNTY

SEQ ID NO: 278 - protein, 36 aa, Artificial Sequence
  Description of Artificial Sequence Synthetic construct
CNTATCATQRLTNFLVHSSHNLGAVLPSTDVGSNTY

SEQ ID NO: 279 - protein, 37 aa, Artificial Sequence
  Description of Artificial Sequence Synthetic construct
KCNTATCATQRLTNFLVRSSHNLGAALSPTDVGSNTY

SEQ ID NO: 280 - protein, 37 aa, Artificial Sequence
  Description of Artificial Sequence Synthetic construct
KCNTATCATQRLTNFLVRSSHNLGAILPPTDVGSNTY

SEQ ID NO: 281 - protein, 37 aa, Artificial Sequence
  Description of Artificial Sequence Synthetic construct
KCNTATCATQRLTNFLVRSSHNLGPALPPTDVGSNTY

SEQ ID NO: 282 - protein, 37 aa, Artificial Sequence
  Description of Artificial Sequence Synthetic construct
KDNTATKATQRLANFLVHSSNNFGAILSSTNVGSNTY

SEQ ID NO: 283 - protein, 37 aa, Artificial Sequence
  Description of Artificial Sequence Synthetic construct
ACNTATCATQRLANFLVHSSNNFGAILSSTNVGSNTY

SEQ ID NO: 284 - protein, 37 aa, Artificial Sequence
  Description of Artificial Sequence Synthetic construct
SCNTATCATQRLANFLVHSSNNFGAILSSTNVGSNTY

SEQ ID NO: 285 - protein, 37 aa, Artificial Sequence
  Description of Artificial Sequence Synthetic construct
KCNTATCATQRLANFLVHSSNNFGAILSPTNVGSNTY

SEQ ID NO: 286 - protein, 37 aa, Artificial Sequence
  Description of Artificial Sequence Synthetic construct
KCNTATCATQRLANFLVHSSNNFGPILPSTNVGSNTY

SEQ ID NO: 287 - protein, 36 aa, Artificial Sequence
  Description of Artificial Sequence Synthetic construct
CNTATCATQRLANFLVHSSNNFGPILPSTNVGSNTY

SEQ ID NO: 288 - protein, 36 aa, Artificial Sequence
  Description of Artificial Sequence Synthetic construct
CNTATCATQRLANFLVHSSNNFGPVLPPSNVGSNTY

SEQ ID NO: 289 - protein, 37 aa, Artificial Sequence [sequence omitted]

SEQ ID NO: 290 - protein, 37 aa, Artificial Sequence; identical to SEQ ID NO: 257
  Description of Artificial Sequence Synthetic construct

SEQ ID NO: 291 - protein, 32 aa, Artificial Sequence
  Description of Artificial Sequence Synthetic construct
KCNTATCVLGRLSQELHRLQTYPRTNTGSNTY

SEQ ID NO: 292 - protein, 32 aa, Artificial Sequence
  Description of Artificial Sequence Synthetic construct
CSNLSTCVLGRLSQELHRLQTYPRTNTGSNTY

SEQ ID NO: 293 - protein, 5 aa, Artificial Sequence
  Description of Artificial Sequence Synthetic peptide
GGXSS

SEQ ID NO: 294 - protein, 6 aa, Artificial Sequence
  Description of Artificial Sequence Synthetic peptide
GGXSSG

SEQ ID NO: 295 - protein, 7 aa, Artificial Sequence
  Description of Artificial Sequence Synthetic peptide
GGXSSGA

SEQ ID NO: 296 - protein, 8 aa, Artificial Sequence
  Description of Artificial Sequence Synthetic peptide
GGXSSGAX

SEQ ID NO: 297 - protein, 9 aa, Artificial Sequence
  Description of Artificial Sequence Synthetic peptide
GGXSSGAXX

SEQ ID NO: 298 - protein, 10 aa, Artificial Sequence
  Description of Artificial Sequence Synthetic peptide
GGXSSGAXXX

SEQ ID NO: 299 - protein, 11 aa, Artificial Sequence
  Description of Artificial Sequence Synthetic peptide
GGXSSGAXXXX

(19) United States
(12) Patent Application Publication
(10) Pub. No.: US 2015/0157725 A1
(43) Pub. Date: Jun. 11, 2015

(54) TRANSMUCOSAL DELIVERY OF PEPTIDES AND PROTEINS

(71) Applicants: AMYLIN PHARMACEUTICALS, LLC, San Diego, CA (US); ASTRAZENECA PHARMACEUTICALS LP, Wilmington, DE (US)

(72) Inventors: John ONG, San Diego, CA (US); Robert JENNINGS, San Diego, CA (US); Christopher RHODES, San Diego, CA (US); Gregg STETSKO, San Diego, CA (US)

(73) Assignees: ASTRAZENECA PHARMACEUTICALS LP; AMYLIN PHARMACEUTICALS, LLC

(21) Appl. No.: 14/570,295

(22) Filed: Dec. 15, 2014

Related U.S. Application Data
(63) Continuation of application No. 11/628,123, filed on Nov. 28, 2006, now abandoned.

(30) Foreign Application Priority Data
May 28, 2004 (US) PCTUS2004017456

(51) Int. Cl. A61K 47/34; A61K 47/38; A61K 47/02; A61K 38/22; A61K 47/18

(52) U.S. Cl. CPC A61K 47/34; A61K 38/2278; A61K 47/183; A61K 47/02; A61K 47/38

(57) ABSTRACT

Provided are methods and compositions for enhancing the transmucosal absorption of bioactive peptides and proteins. More particularly, the invention provides compositions for enhancing the transmucosal absorption of bioactive peptides and proteins, such as exendin-4, PYY, PYY3-36, and GLP-1 and their analogs and derivatives, wherein the compositions comprise an absorption enhancing mixture of a cationic polyamino acid, at least one additional absorption enhancing agent, and a buffer that is compatible with the polyamino acid. Also provided are methods for enhancing the transmucosal absorption and bioavailability of bioactive peptides and proteins using such compositions.

### CROSS-REFERENCE TO RELATED APPLICATIONS

This application is a continuation of U.S. application Ser. No. 11/628,123, filed Nov. 28, 2006, which is a §371 national stage entry of International Application No. PCT/US2005/001440, filed Jan. 12, 2005, which claims the benefit of International Patent Application PCT/US2004/017456 filed May 28, 2004, each of which is incorporated herein by reference in its entirety for all purposes.

### FIELD OF INVENTION

The present invention relates generally to the field of drug delivery. More particularly, the present invention relates to novel methods and compositions for the enhanced transmucosal delivery of bioactive peptides and proteins.

### BACKGROUND

The administration of therapeutically active peptides and proteins has generally been limited to injection due to difficulties in achieving the required bioavailability via alternative, less invasive routes such as oral, transmucosal, or transdermal. For instance, administration by ingestion can result in chemical and enzymatic degradation in the gastrointestinal tract, resulting in a substantial loss of activity and low bioavailability. Transmucosal delivery through absorptive mucous membranes such as oral, buccal, sublingual, eye, nasal, pulmonary, rectal, and vaginal membranes, on the other hand, has the advantage of being noninvasive and of bypassing hepato/gastrointestinal clearance (at least initially). Peptides and proteins, however, are generally not well absorbed through mucosae because of their molecular size and hydrophilicity. In general, enzyme inhibitors and absorption enhancers need to be coadministered for successful transmucousal delivery of bioactive peptides and proteins.

Classes of absorption enhancers used for transmucosal delivery include bile salts and their derivatives, taurodihydrofusidates, mono- and polycarboxylic acids, cyclodextrins, surfactants (especially non-ionic), chelating agents, cationic polymers, lipids and phospholipids (see Davis and Illum, Clin Pharmacokinet., 42:1107-1128, 2003 for a review). Each of these agents exerts its enhancing effects by a different mechanism, and many have been associated with various degrees of adverse effects. Nonetheless, these enhancers have been demonstrated to enhance the absorption and, consequently, bioavailability of peptides and proteins across the mucous membrane.

The nasal cavity provides an attractive route for peptide and protein delivery because of its relatively high permeability and ease of administration. Nasal spray compositions containing a chelating agent such as disodium ethylenediaminetetraacetate, or bile salt have been shown to enhance the absorption of nona- and deca-peptides having LHRH agonist or antagonist activity (U.S. Pat. Nos. 4,476,116 and 5,116,817). A combination of bile salt and dimethyl-β-cyclodextrin has been used to enhance the nasal absorption of parathyroid hormones (U.S. Pat. No. 5,977,070). Lysophospholipids, acylcarnitines and polyoxyethylene(20) sorbitan monooleate (Tween® 80) have also been used as enhancers for the delivery of insulin and calcitonin across mucous membranes (U.S. Pat. Nos. 5,804,212 and 6,440,392). The cationic polysaccharide chitosan, used as powder, nanoparticle, or in solution, has been demonstrated to enhance mucosal absorption of insulin, other peptides and proteins, and vaccines (U.S. Pat. No. 6,391,318; Dyer at al., Pharm. Res., 19:998-1008, 2002; Illum et al., Pharm. Res., 11:1186-1189, 1994; Fernandez-Urrusuno et al., Pharm. Res., 16:1576-1581, 1999). Additionally, bioadhesive agents, such as carbomers and polycarbophil, have been used to increase the residence time and therefore the bioavailability of insulin from a powder dosage form (Callen and Remon, Controlled Rel., 66:215-220, 2000).

The cationic polyamino acid, polylysine, was mentioned in an aerosol formulation for pulmonary and nasal delivery, but no rationale for its function was given (U.S. Pat. No. 6,294,153). Another cationic polyamino acid, poly-L-arginine was reported to enhance the absorption of fluorescein isothiocyanate labeled dextran (Nasume et al., Intl. J. Pharm., 185:1-12, 1999), but no bioactive peptides or proteins were investigated. Other applications for potential uses of cationic polyamino acids to improve transmucosal delivery of molecules can be found in U.S. Pat. Nos. 5,554,388 and 5,788,959; Japanese Patent Applications 1998095738A, 2000281589A; McEwan et al., Biochim. Biophys. Acta, 1148:51-60, 1993; Uchida et al., Exp. Lung Res., 22:85-99, 1996; Natsume et al., Drug Deliv. Systems, 14:21-25, 1999; Miyamoto et al, Intl. J. Pharma., 226:127-138, 2001; Miyamoto et al., Eur. J. Pharma Biopharma., 52:21-30, 2001; Ohtake et al., J. Controlled Res., 82:263-275, 2002 and Ohtake et al., Pharm. Res., 20:1838-1845, 2003. Many of these papers describe the use of cationic polyamino acids to deliver marker molecules such a labeled dextran rather than proteins or peptides. Thus, there remains a need for improved absorption enhancers for use in the transmucosal delivery of bioactive peptides and proteins.

### SUMMARY

Among the several aspects of the invention is provided a pharmaceutical composition for the transmucosal administration of a bioactive peptide or protein of interest comprising the bioactive peptide or protein of interest, an absorption enhancing amount of a cationic polyamino acid, and a compatible buffer that does not cause precipitation of the cationic polyamino acid and has a mono-anionic or neutral net charge at the pH of the composition. The composition is further characterized in that the transmucosal absorption of the bioactive protein or peptide of interest is increased relative to the absorption of the protein or peptide in the absence or substantial absence of the cationic polyamino acid. In one embodiment the absorption of the bioactive protein or peptide is increased at least 2-fold, while in other embodiments it is increased at least 5-fold or at least 10-fold. In one embodiment, the pH of the composition ranges from about pH 3.0 to about pH 8.0, in another embodiment from about pH 3.0 to about pH 6.0, while in another embodiment the pH is between about pH 4.0 and about pH 5.0. In still a further embodiment, the pH of the composition is about pH 4.5. In another embodiment, the compatible buffer comprises glutamic acid, while in other embodiments the compatible buffer comprises acetic acid, aspartic acid, or s-aminocaproic acid. In a further embodiment, the cationic polyamino acid comprises poly-arginine, while in other embodiments the cationic polyamino acid is poly-histidine, poly-lysine or any combination of poly-arginine, poly-histidine and poly-lysine. In one embodiment the cationic polyamino acid or acids has an average molecular weight of between about 10 kDa and about 300 kDa. In another embodiment the polycationic polyamino acid or acids has an average molecular weight between about 10 kDa and about 200 kDa. While in another embodiment, the cationic polyamino acid has an average molecular weight of between about 100 kDa and 200 kDa. In still a further embodiment, the cationic polyamino acid has an average molecular weight between about 140 kDa and about 150 kDa, while in yet another embodiment, the cationic polyamino acid has an average molecular weight of about 141 kDa.

In other embodiments, the composition further comprises a tonicifying agent, a viscosity-increasing agent, a bioadhesive agent, a preservative or any combination of a tonicifying agent, a viscosity-increasing agent, a bioadhesive agent, and a preservative. In one embodiment the tonicifying agent used is selected from sodium chloride, mannitol, sucrose, glucose and any combination of sodium chloride, mannitol, sucrose and glucose, wherein the composition can be hypo-tonic, iso-tonic or hyper-tonic. In another embodiment in which a viscosity-increasing agent is used, the agent can be selected from hydroxypropyl cellulose, hydroxypropyl methylcellulose, methylcellulose with an average molecular weight between about 10 and about 1500 kDa, starch, gums and any combination of the listed viscosity increasing agents. In another embodiment, in which a bioadhesive agent is used, the bioadhesive agent can be selected from carbomer, polycarbophil and any combination of carbomer and polycarbophil. In embodiments utilizing a preservative, the preservative can be selected from benzalkonium chloride, phenylethyl alcohol, methylparaben, ethylparaben, propylparaben, butylparaben, chlorobutanol, benzoic acid, sorbic acid, phenol, m-cresol, alcohol, and any combination of the preservatives listed herein.

In certain embodiments, the cationic polyamino acid is combined with additional absorption enhancers or absorption enhancing agents to further increase the absorption of a bioactive peptide or protein as compared to the absorption enhancement by either the cationic polyamino acid or the other enhancer alone. Examples of additional absorption enhancers include, but not limited to, cationic polysaccharide chitosan, phospholipids such as didecanoyl phosphatidylcholine, a cyclodextrin such as methyl-β-cyclodextrin, hydroxypropyl-β-cyclodextrin, α-cyclodextrin, and γ-cyclodextrin, a chelating agent such as disodium ethylenediaminetetraacetate, a nonionic glycosidic surfactant such as tetradocyl maltoside, sucrose ester surfactants such as alkyl sucrose, a carnitine such as dodecanoyl carnitine and palmitoyl carnitine, and any mixture or combination thereof.

In certain other embodiments, the bioactive protein or peptide is an exendin, an exendin analog or an exendin derivative described herein or known in the art including polymer-modified compounds thereof. In various embodiments the bioactive peptide or protein is exendin-3, exendin-4 or one of the analogs or derivatives described by any of Formulas I, II or III or listed in Table 1. In specific embodiments, the exendin analogs or derivatives include but are not limited to exendin-4 acid, exendin-4 (1-30), exendin-4 (1-30) amide, exendin-4 (1-28) amide, ¹⁴Leu, ²⁵Phe exendin-4 amide, and ¹⁴Leu, ²⁵Phe exendin-4 (1-28) amide.

In other embodiments, the bioactive protein or peptide is GLP-1 or any of the GLP-1 analogs and derivatives listed herein or known in the art including polymer-modified compounds thereof. In still another embodiment, the bioactive protein or peptide is a PYY peptide or an analog or a derivative of a PYY peptide listed herein or known in the art including polymer-modified compounds thereof. In yet another embodiment, the bioactive protein or peptide is amylin or an analog or a derivative of amylin listed herein or known in the art including polymer-modified compounds thereof.

One embodiment provides a pharmaceutical composition for transmucosal administration of a bioactive peptide or protein of interest comprising about 0.01% to about 5.0% (w/v) of the bioactive peptide or protein of interest, such as an exendin, a GLP-1, an amylin, or a PYY peptide as well and analogs of, derivatives of; and polymer-modified exendin, a GLP-1, amylin, and PYY; about 0.01% to about 10.0% (w/v) of a cationic polyamino acid having a molecular weight between about 10 kDa and about 300 kDa; such as poly-arginine, poly-histidine and poly-lysine; and about 0.01% to about 10.0% (w/v) of a compatible buffer, that at the pH of the composition does not cause precipitation of the cationic polyamino acid, and has a mono-anionic or neutral net charge. In one embodiment, the composition has a pH of between about pH 3.0 and 8.0, while in another embodiment, the composition has a pH of between about pH 4.0 and about pH 5.0. Additionally, the transmucosal absorption of the bioactive peptide or protein is increased relative the absorption of said bioactive peptide or protein in the absence of said cationic polyamino acid.

In a particular embodiment is provided a pharmaceutical composition for transmucosal administration comprising about 0.5% (w/v) of exendin-4; about 0.5% (w/v) of poly-L-arginine hydrochloride having an average molecular weight of about 141 kDa; about 0.72% (w/v) sodium chloride; and about 0.56% monosodium glutamate, monohydrate (w/v) at a pH of about 4.5. In an alternative embodiment, this composition further comprises at least one additional absorption enhancing agent.

In another particular embodiment is provided a pharmaceutical composition for transmucosal administration comprising about 0.5% (w/v) of exendin-4; about 1.0% (w/v) of poly-L-arginine hydrochloride having an average molecular weight of about 141 kDa; about 0.72% (w/v) sodium chloride; and about 0.56% monosodium glutamate, monohydrate (w/v) at a pH of about 4.5. In an alternative embodiment, this composition further comprises at least one additional absorption enhancing agent.

Further embodiments provide a method for transmucosal administration of a bioactive peptide or protein comprising contacting a mucosal surface with any of the pharmaceutical compositions described herein for a time sufficient for a therapeutically effective amount of the bioactive peptide or protein of interest to cross the mucosa such that the transmucosal absorption of the bioactive protein or peptide is increased relative to the absorption of the bioactive protein or peptide in the absence or substantial absence of a cationic polyamino acid, such as in the compositions described herein. In one embodiment, the bioactive peptide or protein is an exendin, an exendin analog, or an exendin derivative described herein or known in the art including polymer-modified compounds thereof. In another embodiment, the bioactive peptide or protein is GLP-1, a GLP-1 analog or a GLP-1 derivative described herein or known in the art including polymer-modified compounds thereof. In still another embodiment, the bioactive peptide or protein is a PYY peptide, a PYY peptide analog, or a PYY peptide derivative described herein or known in the art including polymer-modified compounds thereof. In yet another embodiment, the bioactive peptide or protein is amylin, an amylin analog, or an amylin derivative described herein or known in the art including polymer-modified compounds thereof.

Also provided are methods for increasing the bioavailability of a bioactive protein or peptide of interest comprising administering to a subject any of the pharmaceutical compositions described herein for a time sufficient to allow transmucosal absorption of the protein or peptide such that the bioavailability of the bioactive peptide or protein of interest is greater than when the peptide or protein is administered alone, that is in the absence or substantial absence of the cationic polyamino acid. In one embodiment, the method is used to increase the bioavailability of an exendin, an exendin analog, or an exendin derivative described herein or known in the art including polymer-modified compounds thereof. In another embodiment, the method is used to increase the bioavailability of GLP-1, a GLP-1 analog, or a GLP-1 derivative described herein or known in the art, including polymer modified compounds thereof. In yet another embodiment, the method is used to increase the bioavailability of a PYY peptide, a PYY analog, or a PYY derivative described herein or known in the art including polymer-modified compounds thereof. In still another embodiment, the method is used to increase the bioavailability of amylin, an amylin analog, or an amylin derivative described herein or known in the art including polymer-modified compounds thereof.

### BRIEF DESCRIPTION OF THE DRAWINGS

FIG. 1 depicts the bioavailability enhancement of three exendin-4 aqueous solutions containing poly-L-arginine with or without hydroxypropyl methylcellulose as compared to a control exendin-4 solution without poly-L-arginine. Shown are the pharmacokinetic profiles of exendin-4 in Cynomolgus monkeys (n=3) after intranasal doses normalized to 1 μg/kg.

FIG. 2 depicts the area under the plasma curves (AUC) (0-8 hours) of exendin-4 nasal formulations relative to a formulation including 5 mg/mL poly-L-arginine (NF-1). NF-1, NF-2 and NF-3 are the compositions described in Examples 1, 2 and 3, respectively. NF-4 is a control formulation lacking poly-L-arginine.

### DETAILED DESCRIPTION

In one aspect, the present invention teaches the design of novel pharmaceutical compositions for the transmucosal delivery of bioactive peptides and proteins. The novel compositions of the invention may be used to effectively deliver bioactive peptides and proteins systemically to the blood subsequent to transmucosal administration.

More particularly, it has now been found that enhanced transmucosal absorption of bioactive peptides and proteins can be achieved when delivered in conjunction with an absorption enhancing composition comprising a cationic polyamino acid and a buffer which is compatible with the cationic polyamino acid.

Generally, peptides and proteins comprise hydrophobic, hydrophilic, and charged regions which are all capable of interaction with other molecules. As such, one of skill in the art may expect that cationic compounds, such as cationic polyamino acids, would interact with the negative charges of the peptides or proteins. Based on precipitation encountered when cationic polyamino acids are formulated with multi-anionic buffers, such interactions may be expected to result in precipitation or inactivity of the cationic polyamino acid as a permeation enhancer. However, it was unexpectedly discovered that cationic polyamino acids, particularly when formulated with buffers that avoid interaction and/or precipitation of the polyamino acids with bioactive peptides or proteins, actually act as a transmucosal absorption enhancer. Increases in absorption can be at least 1.5-fold, at least 2-fold, at least 5-fold or at least 10 fold greater than that obtained in the absence or substantial absence of the cationic polyamino acid. The extent of the enhanced absorption exceeds what would be normally expected with traditional cationic absorption enhancers such as chitosan not in combination with a cationic polyamino acid. Further, this enhanced transmucosal absorption results in an unexpected improvement in bioavailability of greater than 1.5-fold, greater than 2-fold, greater than 5-fold or greater than 10-fold compared to transmucosal delivery in the absence or substantial absence of the absorption enhancing compositions described herein. It will be apparent to those skilled in the art that the exact increase in absorption or bioavailability may vary with known factors such as the size of the protein, the method of administration, the concentration of the bioactive protein or peptide, the amount of composition applied, and the particular mucosal surface to which the composition is applied.

Other aspects relate to methods for enhancing the transmucosal absorption of bioactive peptides and proteins, and methods for improving the bioavailability of bioactive peptides and proteins when administered via transmucosal delivery. The pharmaceutical compositions can be delivered to the mucous membrane absorption site by any means known in the art, for example, dropping or spraying from a bottle into the eye, nasal, buccal, or sublingual cavity; by aerosolizing from an inhaler into the pulmonary region; as well as by applying a tablet, capsule, permeable/soluble matrix, or other known dosage forms to the buccal, sublingual, rectal, or vaginal areas.

The pharmaceutical compositions described herein that provide enhanced transmucosal absorption generally comprise a bioactive peptide or protein in combination with an absorption enhancing mixture comprising a cationic polyamino acid and a buffer that is compatible with the cationic polyamino acid. Optionally, the pharmaceutical compositions of the invention may also include one or more excipients such as agent(s) to render the solution compatible with body tissue; viscosity-increasing agent(s), bioadhesive agents, preservative(s), and the like.

The bioactive peptides or proteins of the invention include peptides or proteins that are inherently compatible or formulated to be compatible with the cationic polyamino acids of the invention, i.e., those bioactive peptides and proteins which do not interact with or cause precipitation of the cationic polyamino acid when in solution. In one embodiment the peptide or protein has the same net charge as the polyamino acid at the pH of the composition. For example, at the pH of the composition both the protein and the polyamino acid have a net positive charge. In this situation, it is not necessary that the magnitude of the charge be identical, but only that the net charge be the same.

The bioactive peptides or proteins used in the composition can be any bioactive protein or peptide known in the art. In one embodiment the bioactive peptides and proteins comprise exendins, exendin analogs and exendin derivatives. Examples of suitable exendins include exendin-3, exendin-4, exendin-4 acid, exendin-4 (1-30), exendin-4 (1-30) amide, exendin-4 (1-28), exendin-4 (1-28) amide, ¹⁴Leu, ²⁵Phe exendin-4 amide, and ¹⁴Leu, ²⁵Phe exendin-4 (1-28) amide as well as other bioactive exendins known in the art such as those described in International Patent Application Publication Nos. WO 99/07404, WO 99/25727, WO 99/25728, and WO 01/04156; US Patent Application Publication Nos. US 2003-0087820, US 2002-137666 and US 2003-087821; and U.S. Pat. No. 6,528,486, all of which are herein incorporated by reference in their entireties and in particular the exendin-related sequences contained therein.

Exendins that can be used in the compositions disclosed herein include those described by Formula I (SEQ ID No. 3) which is as follows:

Xaa₁ Xaa₂ Xaa₃ Gly Thr Xaa₆ Xaa₇ Xaa₈ Xaa₉ Xaa₁₀ Ser Lys Gln Xaa₁₄ Glu Glu Glu
Ala Val Arg Leu Xaa₂₂ Xaa₂₃ Xaa₂₄ Xaa₂₅ Leu Lys Asn Gly Gly Xaa₃₁ Ser Ser Gly
Ala Xaa³⁶ Xaa³⁷ Xaa³⁸ Xaa³⁹;

where:
Xaa₁ is His, Arg or Tyr;
Xaa₂ is Ser, Gly, Ala or Thr;
Xaa₃ is Asp or Glu;
Xaa₆ is Phe, Tyr or naphthylalanine;
Xaa₇ is Thr or Ser;
Xaa₈ is Ser or Thr;
Xaa₉ is Asp or Glu;
Xaa₁₀ is Leu, Ile, Val, pentylglycine or Met;
Xaa₁₄ is Leu, Ile, pentylglycine, Val or Met;
Xaa₂₂ is Phe, Tyr or naphthylalanine;
Xaa₂₃ is Ile, Val, Leu, pentylglycine, tert-butylglycine or Met;
Xaa₂₄ is Glu or Asp;
Xaa₂₅ is Trp, Phe, Tyr, or naphthylalanine;
Xaa₃₁, Xaa₃₆, Xaa₃₇, and Xaa₃₈ are independently Pro, homoproline, 3Hyp, 4Hyp, thioproline, N-alkylglycine, N-alkylpentylglycine or N-alkylalanine;
Xaa₃₉ is Ser, Thr or Tyr; and
wherein the terminal amino acid is optionally amidated

Examples of additional exendins that can be used in the compositions disclosed herein include those described by Formula II (SEQ ID No. 4) which is as follows:

Xaa₁ Xaa₂ Xaa₃ Gly Xaa₅ Xaa₆ Xaa₇ Xaa₈ Xaa₉ Xaa₁₀ Xaa₁₁ Xaa₁₂ Xaa₁₃ Xaa₁₄ Xaa₁₅
Xaa₁₆ Xaa₁₇ Ala Xaa₁₉ Xaa₂₀ Xaa₂₁ Xaa₂₂ Xaa₂₃ Xaa₂₄ Xaa₂₅ Xaa₂₆ Xaa₂₇ Xaa₂₈-Z1;

where
Xaa₁ is His, Arg or Tyr,
Xaa₂ is Ser, Gly, Ala or Thr,
Xaa₃ is Ala, Asp or Glu;
Xaa₅ is Ala or Thr,
Xaa₆ is Ala, Phe, Tyr or naphthylalanine;
Xaa₇ is Thr or Ser;
Xaa₈ is Ala, Ser or Thr,
Xaa₉ is Asp or Glu;
Xaa₁₀ is Ala, Leu, le, Val, pentylglycine or Met;
Xaa₁₁ is Ala or Ser;
Xaa₁₂ is Ala or Lys;
Xaa₁₃ is Ala or Gin;
Xaa₁₄ is Ala, Leu, Ile, pentylglycine, Val or Met;
Xaa₁₅ is Ala or Glu;
Xaa₁₆ is Ala or Glu;
Xaa₁₇ is Ala or Glu;
Xaa₁₉ is Ala or Val;
Xaa₂₀ is Ala or Arg;
Xaa₂₁ is Ala or Leu;
Xaa₂₂ is Ala, Phe, Tyr or naphthylalanine;
Xaa₂₃ is Ile, Val, Leu, pentylglycine, tert-butylglycine or Met;
Xaa₂₄ is Ala, Glu or Asp;
Xaa₂₅ is Ala, Trp, Phe, Tyr or naphthylalanine;
Xaa₂₆ is Ala or Leu;
Xaa₂₇ is Ala or Lys;
Xaa₂₈ is Ala or Asn;
Z₁ is —OH,

—NH₂,
Gly,
Gly Gly,
Gly Gly Xaa₃₁
Gly Gly Xaa₃₁ Ser,
(SEQ ID NO: 293)
Gly Gly Xaa₃₁ Ser Ser,
(SEQ ID NO: 294)
Gly Gly Xaa₃₁ Ser Ser Gly,
(SEQ ID NO: 295)
Gly Gly Xaa₃₁ Ser Ser Gly Ala,
(SEQ ID NO: 296)
Gly Gly Xaa₃₁ Ser Ser Gly Ala Xaa₃₆,
(SEQ ID NO: 297)
Gly Gly Xaa₃₁ Ser Ser Gly Ala Xaa₃₆ Xaa₃₇,
or
(SEQ ID NO: 298)
Gly Gly Xaa₃₁ Ser Ser Gly Ala Xaa₃₆ Xaa₃₇ Xaa₃₈;

Xaa₃₁ Xaa₃₆, Xaa₃₇ and Xaa₃₈ are independently Pro, homoproline, 3Hyp, 4Hyp, thioproline, N-alkylglycine, N-alkylpentylglycine or N-alkylalanine; and
the terminal amino acid is optionally amidated;
provided that no more than three of Xaa₃, Xaa₅, Xaa₆, Xaa₈, Xaa₁₀, Xaa₁₁, Xaa₁₂, Xaa₁₃, Xaa₁₄, Xaa₁₅, Xaa₁₆, Xaa₁₇, Xaa₁₉, Xaa₂₀, Xaa₂₁, Xaa₂₄, Xaa₂₅, Xaa₂₆, Xaa₂₇ and Xaa₂₈ are Ala

Additional examples of exendins that are suitable for use in the compositions disclosed herein are those described by Formula III (SEQ ID No. 5) which is as follows:

Xaa₁ Xaa₂ Xaa₃ Xaa₄ Xaa₅ Xaa₆ Xaa₇ Xaa₈ Xaa₉ Xaa₁₀ Xaa₁₁ Xaa₁₂ Xaa₁₃ Xaa₁₄ Xaa₁₅
Xaa₁₆ Xaa₁₇ Ala Xaa₁₉ Xaa₂₀ Xaa₂₁ Xaa₂₂ Xaa₂₃ Xaa₂₄ Xaa₂₅ Xaa₂₆ Xaa₂₇ Xaa₂₈-Z₁;

wherein
Xaa₁ is His, Arg, Tyr, Ala, Norval, Val or Norleu;
Xaa₂ is Ser, Gly, Ala or Thr;
Xaa₃ is Ala, Asp or Glu;
Xaa₄ is Ala, Norval, Val, Norleu or Gly;
Xaa₅ is Ala or Thr;
Xaa₆ is Ala, Phe, Tyr or naphthylalanine;
Xaa₇ is Thr or Ser;
Xaa₈ is Ala, Ser or Thr;
Xaa₉ is Ala, Norval, Val, Norleu, Asp or Glu;
Xaa₁₀ is Ala, Leu, Ile, Val, pentylglycine or Met;
Xaa₁₁ is Ala or Ser;
Xaa₁₂ is Ala or Lys;
Xaa₁₃ is Ala or Gin;
Xaa₁₄ is Ala, Lou, Ile, pentylglycine, Val or Met;
Xaa₁₅ is Ala or Glu;
Xaa₁₆ is Ala or Glu;
Xaa₁₇ is Ala or Glu;
Xaa₁₉ is Ala or Val;
Xaa₂₀ is Ala or Arg;
Xaa₂₁ is Ala or Leu;
Xaa₂₂ is Phe, Tyr or naphthylalanine;
Xaa₂₃ is Ile, Val, Leu, pentylglycine, tert-butylglycine or Met;
Xaa₂₄ is Ala, Glu or Asp;
Xaa₂₅ is Ala, Trp, Phe, Tyr or naphthylalanine;
Xaa₂₆ is Ala or Leu;
Xaa₂₇ is Ala or Lys;
Xaa₂₈ is Ala or Asn;
Z₁ is —OH,

—NH₂
Gly,
Gly Gly,
Gly Gly Xaa₃₁,
Gly Gly Xaa₃₁ Ser,
(SEQ ID NO: 293)
Gly Gly Xaa₃₁ Ser Ser,
(SEQ ID NO: 294)
Gly Gly Xaa₃₁ Ser Ser Gly,
(SEQ ID NO: 295)
Gly Gly Xaa₃₁ Ser Ser Gly Ala,
(SEQ ID NO: 296)
Gly Gly Xaa₃₁ Ser Ser Gly Ala Xaa₃₆,
(SEQ ID NO: 297)
Gly Gly Xaa₃₁ Ser Ser Gly Ala Xaa₃₆ Xaa₃₇,
(SEQ ID NO: 298)
Gly Gly Xaa₃₁ Ser Ser Gly Ala Xaa₃₆ Xaa₃₇ Xaa₃₈,
or
or
(SEQ ID NO: 299)
Gly Gly Xaa₃₁ Ser Ser Gly Ala Xaa₃₆ Xaa₃₇ Xaa₃₈
Xaa₃₉;

where:
   Xaa₃₁, Xaa₃₆, Xaa₃₇ and Xaa₃₈ are independently Pro, homoproline, 3Hyp, 4Hyp, thioproline, N-alkylglycine, N-alkylpentylglycine or N-alkylalanine;
   Xaa₃₉ is Ser, Thr or Tyr; and
   the terminal amino acid is optionally amidated;
   provided that no more than three of Xaa₃, Xaa₄, Xaa₅, Xaa₆, Xaa₈, Xaa₉, Xaa₁₀, Xaa₁₁, Xaa₁₂, Xaa₁₃, Xaa₁₄, Xaa₁₅, Xaa₁₆, Xaa₁₆, Xaa₁₇, Xaa₁₉, Xaa₂₀, Xaa₂₁, Xaa₂₄, Xaa₂₅, Xaa₂₆, Xaa₂₇ and Xaa₂₈ are Ala;
and provided also that, if Xaa₁ is His, Arg or Tyr, then at least one of Xaa₃, Xaa₄ and Xaa₉ is Ala.

Examples of particular exendins, exendin analogs and exendin derivatives that can be used in the compositions described herein, include, but are not limited to those describe in Table 1. In one embodiment, the bioactive peptide or protein is exendin-4.

TABLE 1
Exendins, Exendin Analogs and Exendin Derivatives

| SEQ | |
|---|---|
| ID | |
| NO | Sequence |
| 1 | His Ser Asp Gly Thr Phe Thr Ser Asp Leu Ser Lys Gln Met Glu Glu Glu Ala Val Arg Leu |
| | Phe Ile Glu Trp Leu Lys Asn Gly Gly Pro Ser Ser Gly Ala Pro Pro Pro Ser |
| 2 | His Gly Glu Gly Thr Phe Thr Ser Asp Leu Ser Lys Gln Met Glu Glu Glu Ala Val Arg Leu |
| | Phe Ile Glu Trp Leu Lys Asn Gly Gly Pro Ser Ser Gly Ala Pro Pro Pro Ser |
| 6 | His Gly Glu Gly Thr Phe Thr Ser Asp Leu Ser Lys Gln Met Glu Glu Glu Ala Val Arg Leu Phe |
| | Ile Glu Trp Leu Lys Asn Gly Gly |
| 7 | His Gly Glu Gly Thr Phe Thr Ser Asp Leu Ser Lys Gln Met Glu Glu Glu Ala Val Arg Leu Phe |
| | Ile Glu Trp Leu Lys Asn Gly Gly-NH₂ |
| 8 | His Gly Glu Gly Thr Phe Thr Ser Asp Leu Ser Lys Gln Leu Glu Glu Glu Ala Val Arg Leu Ala |
| | Ile Glu Phe Leu Lys Asn-NH₂ |
| 9 | His Gly Glu Gly Thr Phe Thr Ser Asp Leu Ser Lys Gln Leu Glu Glu Glu Ala Val Arg Leu Phe |
| | Ile Glu Phe Leu Lys Asn Gly Gly Pro Ser Ser Gly Ala Pro Pro Pro Ser-NH₂ |
| 10 | His Gly Glu Gly Thr Phe Thr Ser Asp Leu Ser Lys Gln Leu Glu Glu Glu Ala Val Arg Leu |
| | Phe Ile Glu Trp Leu Lys Asn Gly Gly Pro Ser Ser Gly Ala Pro Pro Pro Ser NH₂ |
| 11 | His Gly Glu Gly Thr Phe Thr Ser Asp Leu Ser Lys Gln Met Glu Glu Glu Ala Val Arg Leu |
| | Phe Ile Glu Phe Leu Lys Asn Gly Gly Pro Ser Ser Gly Ala Pro Pro Pro Ser NH₂ |
| 12 | Tyr Gly Glu Gly Thr Phe Thr Ser Asp Leu Ser Lys Gln Met Glu Glu Glu Ala Val Arg Leu |
| | Phe Ile Glu Trp Leu Lys Asn Gly Gly Pro Ser Ser Gly Ala Pro Pro Pro Ser NH₂ |
| 13 | His Gly Glu Gly Thr Phe Thr Ser Asp Leu Ser Lys Gln Met Glu Glu Glu Ala Val Arg Leu |
| | Phe Ile Glu Trp Leu Lys Asn Gly Gly Pro Ser Ser Gly Ala Pro Pro Pro Tyr NH₂ |
| 14 | His Gly Asp Gly Thr Phe Thr Ser Asp Leu Ser Lys Gln Met Glu Glu Glu Ala Val Arg Leu |
| | Phe Ile Glu Trp Leu Lys Asn Gly Gly Pro Ser Ser GI Ala Pro Pro Pro Ser NH₂ |
| 15 | His Gly Glu Gly Thr napthylAla Thr Ser Asp Leu Ser Lys Gln Met Glu Glu Glu Ala Val Arg |
| | Leu Phe Ile Glu Trp Leu Lys Asn Gly Gly Pro Ser Ser Gly Ala Pro Pro Pro Ser NH₂ |
| 16 | His Gly Glu Gly Thr Phe Ser Ser Asp Leu Ser Lys Gln Met Glu Glu Glu Ala Val Arg Leu |
| | Phe Ile Glu Trp Leu Lys Asn Gly Gly Pro Ser Ser Gly Ala Pro Pro Pro Ser NH₂ |
| 17 | His Gly Glu Gly Thr Phe Ser Thr Asp Leu Ser Lys Gln Met Glu Glu Glu Ala Val Arg Leu |
| | Phe Ile Glu Trp Leu Lys Asn Gly Gly Pro Ser Ser Gly Ala Pro Pro Pro Ser NH₂ |
| 18 | His Gly Glu Gly Thr Phe Thr Thr Asp Leu Ser Lys Gln Met Glu Glu Glu Ala Val Arg Leu |
| | Phe Ile Glu Trp Leu Lys Asn Gly Gly Pro Ser Ser Gly Ala Pro Pro Pro Ser NH₂ |
| 19 | His Gly Giu Gly Thr Phe Thr Set Glu Leu Ser Lys Gln Met Glu Glu Glu Ala Val Arg Leu |
| | Phe Ile Glu Trp Leu Lys Asn Gly Gly Pro Set Set Gly Ala Pro Pro Pro Ser NH₂ |
| 20 | His Gly Glu Gly Thr the Thr Ser Asp pentylGly Ser Lys Gln Met Glu Glu Glu Ala Val Arg |
| | Leu Phe Ile Glu Trp Leu Lys Asn Gly Gly Pro Ser Ser Gly Ala Pro Pro Pro Ser NH |
| 21 | His Gly Glu Gly Thr Phe Thr Set Asp pentylGly Set Lys Gln Leu Glu Glu Glu Ala Val Arg |
| | Leu Phe Ile Glu Phe Leu Lys Asn Gly Gly Pro Ser Serr Gly Ala Pro Pro Pro Ser NH₂ |
| 22 | His Gly Glu Gly Thr Phe Thr Ser Asp Leu Set Lys Gln pentylGly Glu Glu Glu Ala Val Arg |
| | Leu Phe Ile Glu Trp Leu Lys Asn Gly Gly Pro Ser Ser Gly Ala Pro Pro Pro Ser NH₂ |
| 23 | His Gly Glu Gly Thr Phe Thr Ser Asp Leu Set Lys Gln pentylGly Glu Glu Glu Ala Val Arg |
| | Leu Phe Ile Glu Phe Leu Lys Asn Gly Gly Pro Ser Ser Gly Ala Pro Pro Pro Ser NH₂ |
| 24 | His Gly Glu Gly Thr Phe Thr Ser Asp Leu Ser Lys Gln Met Glu Glu Glu Ala Val Arg Leu |
| | napthylAla Ile Glu Trp Leu Lys Asn Gly Gly Pro Ser Ser Gly Ala Pro Pro Pro Ser NH₂ |
| 25 | His Gly Glu Gly Thr Phe Thr Ser Asp Leu Ser Lys Gln Met Glu Glu Glu Ala Val Arg Leu |
| | Phe Val Glu Trp Leu Lys Asn Gly Gly Pro Ser Ser Gly Ala Pro Pro Pro Ser NH₂ |
| 26 | His Gly Glu Gly Thr Phe Thr Ser Asp Leu Ser Lys Gln Leu Glu Glu Glu Ala Val Arg Leu |
| | Phe Val Glu Phe Leu Lys Asn Gly Gly Pro Ser Ser Gly Ala Pro Pro Pro Ser NH₂ |
| 27 | His Gly Glu Gly Thr Phe Thr Ser Asp Leu Ser Lys Gln Met Glu Glu Glu Ala Val Arg Leu |
| | Phe tbutylGly Glu Trp Leu Lys Asn Gly Gly Pro Ser Ser Gly Ala Pro Pro Pro Ser NH₂ |
| 28 | His Gly Glu Gly Thr Phe Thr Ser Asp Leu Ser Lys Gln Leu Glu Glu Glu Ala Val Arg Leu |
| | Phe tbutylGly Glu Phe Leu Lys Asn Gly Gly Pro Ser Ser Gly Ala Pro Pro Pro Set NH₂ |
| 29 | His Gly Glu Gly Thr Phe Thr Ser Asp Leu Ser Lys Gln Met Glu Glu Glu Ala Val Arg Leu |
| | Phe Ile Asp Trp Leu Lys Asn Gly Gly Pro Ser Ser Gly Ala Pro Pro Pro Ser NH₂ |
| 30 | His Ala Glu Gly Thr Phe Thr Ser Asp Leu Ser Lys Gln Leu Glu Glu Glu Ala Val Arg Leu |
| | Phe Ile Glu Phe Leu Lys Asn Gly Gly Pro Sex- Ser. Gly Ala Pro Pro Pro Ser NH₂ |
| 31 | His Gly Glu Gly Thr Phe Thr Ser Asp Leu Ser Lys Gln Met Glu Glu Glu Ala Val Arg Leu |
| | Phe Ile Glu Trp Leu Lys Asn Gly Gly thioPro Ser. Ser Gly Ala thioPro thioPro thioPro |
| | Set NH₂ |
| 32 | His Gly Glu Gly Thr Phe Thr Ser Asp Leu Ser Lys Gln Met Glu Glu Glu Ala Val Arg Leu |
| | Phe Ile Glu Trp Leu Lys Asn Gly Gly Pro Ser Ser Gly Ala thioPro thioPro thioPro Ser |
| | NH₂ |
| 33 | His Gly Glu Gly Thr Phe Thr Ser Asp Leu Ser Lys Gln Met Glu Glu Glu Ala Val Arg Leu |
| | Phe Ile Glu Trp Leu Lys Asn Gly Gly homoPro Ser Ser Gly Ala homoPro homoPro homoPro |
| | Ser NH₂ |
| 34 | His Gly Glu Gly Thr Phe Thr Set Asp Leu Ser Lys Gln Met Glu Glu Glu Ala Val Arg Leu |
| | Phe Ile Glu Trp Leu Lys Asn Gly Gly Pro Ser Ser Gly Ala homoPro homoPro homoPro Ser |
| | NH₂ |
| 35 | His Gly Glu Gly Thr Phe Thr Ser Asp Leu Ser Lys Gln Leu Glu Glu Glu Ala Val Arg Leu |
| | Phe Ile Glu Phe Leu Lys Asn Gly Gly thioPro Ser Ser Gly Ala thioPro thioPro thioPro |
| | Set NH₂ |
| 36 | His Gly Glu Gly Thr Phe Thr Ser Asp Leu Ser Lys Gln Leu Glu Glu Glu Ala Val Arg Leu |
| | Phe Ile Glu Phe Leu Lys Asn Gly Gly homoPro Ser Ser Gly Ala homoPro homoPro homoPro |
| | Ser NH₂ |
| 37 | His Gly Glu Gly Thr Phe Thr Set Asp Leu Ser Lys Gln Met Glu Glu Glu Ala Val Arg Leu |
| | Phe Ile Glu Trp Leu Lys Asn Gly Gly NmethylAla Ser Ser Gly Ala NmethylAla NmethylAla |
| | NmethylAla Ser NH₂ |
| 38 | His Gly Glu Gly Thr Phe Thr Ser Asp Leu Ser Lys Gln Met Glu Glu Glu Ala Val Arg Leu |
| | Phe Ile Glu Trp Leu Lys Asn Gly Gly Pro Ser Ser Gly Ala NmethylAla NmethylAla |
| | NmethylAla Ser NH₂ |
| 39 | His Gly Glu Gly Thr Phe Thr Ser Asp Leu Ser Lys Gln Leu Glu Glu Gln Ala Val Arg Leu |
| | Phe Ile Giu Phe Leu Lys Asn Gly Gly NmethylAla Ser Ser Gly Ala NmethylAla NmethylAla |
| | NmethylAla Ser NH₂ |
| 40 | His Gly Glu Gly Thr Phe Thr Ser Asp Leu Set Lys Gln Met Glu Glu Glu Ala Val Arg Leu Phe |
| | Ile Glu Trp Leu Lye Asn-NH₂ |
| 41 | His Gly Glu Gly Thr Phe Thr Ser Asp Leu Ser Lys Gln Leu Glu Glu Glu Ala Val Arg Leu Phe |
| | Ile Glu Phe Leu Lys Asn-NH₂ |
| 42 | His Ala Glu Gly Thr Phe Thr Ser Asp Leu Ser Lys Gln Leu Glu Gln Gln Ala Val Arg Leu |
| | Phe Ile Glu Phe Leu Lys Asn-NH₂ |
| 43 | His Gly Glu Gly Ala Phe Thr Ser Asp Leu Ser Lys Gln Leu Glu Glu Glu Ala Val Arg Leu |
| | Phe Ile Glu Phe Leu Lys Asn-NH₂ |
| 44 | His Gly Gln Gly Thr Ala Thr Ser Asp Leu Ser Lys Gln Leu Glu Glu Glu Ala Val Arg Leu |
| | Phe Ile Gln Phe Leu Lys Asn-NH₂ |
| 45 | His Gly Glu Gly Thr Phe Thr Ala Asp Leu Ser Lys Gln Leu Glu Glu Glu Ala Val Arg Leu |
| | Phe Ile Gln Phe Leu Lys Asn-NH₂ |
| 46 | His Gly Glu Gly Thr Phe Thr Ser Asp Ala Ser Lye Gln Leu Glu Glu Glu Ala Val Arg Leu |
| | Phe Ile Glu Phe Len Lys Asn-NH₂ |
| 47 | His Gly Glu my Thr Phe Thr Set Asp Leu Ala Lys Gln Leu Glu Gln Glu Ala Val Arg Leu |
| | Phe Ile Glu Phe Leu Lys Asn-NH₂ |
| 48 | His Gly Glu Gly Thr Phe Thr Ser Asp Leu Ser Ala Gln Leu Glu Glu Glu Ala Val Arg Leu |
| | Phe Ile Glu Phe Leu Lys Asn-NH₂ |
| 49 | His Gly Glu Gly Thr Phe Thr Ser Asp Leu Ser Lys Ala Leu Glu Glu Glu Ala Val Arg Leu |
| | Phe Ile Glu Phe Leu Lys Asn-NH₂ |
| 50 | His Gly Glu Gly Thr Phe Thr Ser Asp Leu Ser Lys Gln Ala Giu Glu Glu Ala Val Arg Leu |
| | Phe Ile Glu Phe Leu Lys Asn-NH₂ |
| 51 | His Gly Glu Gly Thr Phe Thr Ser Asp Leu Ser Lys Gln Leu Ala Glu Glu Ala Val Arg Leu |
| | Phe Ile Glu Phe Leu Lys Asn-NH₂ |
| 52 | His Gly Glu Gly Thr Phe Thr Ser Asp Leu Ser Lys Gln Leu Glu Ala Glu Ala Val Arg Leu |
| | Phe Ile Glu Phe Leu Lys Asn-NH₂ |
| 53 | His Gly Glu Gly Thr Phe Thr Ser Asp Leu Ser Lys Gln Leu Glu Glu All Ala Val Arg Leu |
| | Phe Ile Glu Phe Leu Lys Asn-NH₂ |
| 54 | His Gly Glu Gly Thr Phe Thr Set Asp Leu Ser Lys Gln Leu Glu Glu Glu Ala Ala Arg Leu |
| | Phe Ile Glu Phe Leu Lys Asn-NH₂ |
| 55 | His Gly Glu Gly Thr Phe Thr Ser Asp Leu Ser Lys Gln Leu Glu Glu Glu Ala Val Ala Leu |
| | Phe Ile Glu Phe Leu Lys Asn-NH₂ |
| 56 | His Gly Glu Gly Thr Phe Thr Ser Asp Leu Ser Lys Gln Leu Glu Glu Glu Ala Val Arg Ala |
| | Phe Ile Glu Phe Leu Lys Asn-NH₂ |
| 57 | His Gly Glu Gly Thr Phe Thr Ser Asp Leu Ser Lys Gln Leu Glu Glu Glu Ala Val Arg Leu |
| | Phe Ile Ala Phe Leu Lys Asn-NH₂ |
| 58 | His Gly Glu Gly Thr Phe Thr Ser Asp Leu Ser Lys Gln Leu Glu Glu Glu Ala Val Arg Leu |
| | Phe Ile Glu Ala Leu Lys Asn-NH₂ |
| 59 | His Gly Glu Gly Thr Phe Thr Ser Asp Leu Ser Lys Gln Leu Glu Glu Glu Ala Val Arg Leu |
| | Phe Ile Glu Phe Ala Lys Asn-NE₂ |
| 60 | His Gly Glu Gly Thr Phe Thr Ser Asp Leu Ser Lys Gln Leu Glu Glu Glu Ala Val Arg Leu |
| | Phe Ile Glu Phe Leu Ala Asn-NE₂ |
| 61 | His Gly Glu Gly Thr Phe Thr Ser Asp Leu Ser Lys Gln Leu Glu Glu Glu Ala Val Arg Leu |
| | Phe Ile Glu Phe Leu Lys Ala-NH₂ |
| 62 | His Gly Glu Gly Thr Phe Thr Ser Asp Leu Ser Lys Gln Met Glu Glu Glu Ala Val Arg Leu |
| | Phe Ile Glu Trp Leu Lys Asn Gly Gly Pro Ser Ser Gly Ala Pro Pro Pro-NH₂ |
| 63 | His Gly Glu Gly Thr Phe Thr Ser Asp Leu Ser Lys Gln Leu Glu Glu Glu Ala Val Arg Leu |
| | Phe Ile Glu Phe Leu Lys Asn Gly Gly Pro Ser Ser Gly Ala Pro Pro Pro-NH₂ |
| 64 | His Gly Glu Gly Thr Phe Thr Ser Asp Leu Ser Lys Gln Met Glu Glu Glu Ala Val Arg Leu |
| | Phe Ile Glu Trp Leu Lys Asn Gly Gly Pro Ser Ser Gly Ala Pro Pro-NH₂ |
| 65 | His Gly Glu Gly Thr Phe Thr Ser Asp Leu Ser Lys Gln Leu Glu Glu Glu Ala Val Arg Leu |
| | Phe Ile Glu Phe Leu Lys Asn Gly Gly Pro Ser Ser Gly Ala Pro Pro-NH₂ |
| 66 | His Gly Glu Gly Thr Phe Thr Ser Asp Leu Ser Lys Gln Met Glu Glu Glu Ala Val Arg Leu |
| | Phe Ile Giu Trp Leu Lys Asn Gly Gly Pro Ser Ser Gly Ala Pro-NH₂ |
| 67 | His Gly Glu Gly Thr Phe Thr Ser Asp Leu Ser Lys Gln Leu Glu Glu Glu Ala Val Arg Leu |
| | Phe Ile Glu Phe Leu Lys Asn Gly Gly Pro Ser Ser Gly Ala Pro-NH₂ |
| 68 | His Gly Glu Gly Thr Phe Thr Ser Asp Leu Ser Lys Gln Met Glu Glu Glu Ala Val Arg Leu |
| | Phe Ile Glu Trp Leu Lys Asn Gly Gly Pro Ser Ser Gly Ala-NH₂ |
| 69 | His Gly Glu Gly Thr Phe Thr Ser Asp Leu Ser Lys Gln Leu Glu Glu Glu Ala Val Arg Leu |
| | Phe Ile Glu Phe Leu Lys Asn Gly Gly Pro Ser Ser Gly Ala-NH₂ |
| 70 | His Gly Glu Gly Thr Phe Thr Ser Asp Leu Ser Lys Gln Met Glu Glu Glu Ala Val Arg Leu |
| | Phe Ile Glu Trp Leu Lys Asn Gly Gly Pro Ser Ser Gly-NH₂ |
| 71 | His Gly Glu Gly Thr Phe Thr Ser Asp Leu Ser Lys Gln Leu Glu Glu Glu Ala Val Arg Leu |
| | Phe lie Glu Phe Leu Lys Asn Gly Gly Pro Ser Ser Gly-NH₂ |
| 72 | His Gly Glu Gly Thr Phe Thr Ser Asp Leu Ser Lys Gln Met Glu Glu Glu Ala Val Arg Leu |
| | Phe Ile Glu Trp Leu Lys Asn Gly Gly Pro Ser Ser-NH₂ |
| 73 | His Gly Glu Gly Thr Phe Thr Ser Asp Leu Ser Lys Gln Leu Glu Glu Glu Ala Val Arg Leu |
| | Phe Ile Glu Phe Leu Lys Asn Gly Gly Pro SerSer-NH₂ |
| 74 | His Gly Glu Gly Thr Phe Thr Ser Asp Leu Ser Lys Gln Met Glu Glu Glu Ala Val Arg Leu |
| | Phe Ile Glu Trp Leu Lys Asn Gly Gly Pro Ser-NH₂ |
| 75 | His Gly Glu Gly Thr Phe Thr Ser Asp Leu Ser Lys Gln Leu Glu Glu Glu Ala Val Arg Leu |
| | Phe Ile Glu Phe Leu Lys Asn Gly Gly Pro Ser-NH₂ |
| 76 | His Gly Glu Gly Thr Phe Thr Ser Asp Leu Ser Lys Gln Met Glu Glu Glu Ala Val Arg Leu |
| | Phe Ile Glu Trp Leu Lys Asn Gly Gly Pro-NH₂ |
| 77 | His Gly Glu Gly Thr Phe Thr Ser Asp Leu Ser Lys Gln Leu Glu Glu Glu Ala Val Arg Leu |
| | Phe Ile Glu Phe Leu Lys Asn Gly Gly Pro-NH₂ |
| 78 | His Gly Glu Gly Thr Phe Thr Ser Asp Leu Ser Lys Gln Leu Glu Glu Glu Ala Val Arg Leu |
| | Phe Ile Glu Phe Leu Lys Asn Gly Gly-NH₂ |
| 79 | His Gly Glu Gly Thr Phe Thr Ser Asp Leu Ser Lys Gln Met Glu Glu Glu Ala Val Arg Leu |
| | Phe Ile Glu Trp Leu Lys Asn Gly-NH₂ |
| 80 | His Gly Glu Gly Thr Phe Thr Ser Asp Leu Ser Lys Gln Leu Glu Glu Glu Ala Val Arg Leu |
| | Phe Ile Glu Phe Leu Lys Asn Gly-NH₂ |
| 81 | His Gly Glu Gly Thr Phe Thr Ser Asp Leu Ser Lys Gln Met Glu Glu Glu Ala Val Arg Leu |
| | Phe Ile Glu Trp Leu Lys Asn Gly Gly thioPro Ser Ser Gly Ala thioPro thioPro thioPro- |
| | NH₂ |
| 82 | His Gly Glu Gly Thr Phe Thr Ser Asp Leu Ser Lys Gln Met Glu Glu Glu Ala Val Arg Leu |
| | Phe Ile Glu Trp Leu Lys Asn Gly Gly Pro Set Ser Gi Ala thioPro thioPro thioPro-NH₂ |
| 83 | His Gly Glu Gly Thr Phe Thr Set Asp Leu Ser. Lys Gln Met Glu Glu Glu Ala Val Arg Leu |
| | Phe Ile Glu Trp Leu Lys Asn G1 Gly NMeala Ser Ser Gly Ala Pro Pro-NH₂ |
| 84 | His Gly Glu Gly Thr Phe Thr Set Asp Leu Ser Lys Gln Met Glu Glu Glu Ala Val Arg Leu |
| | Phe Ile Glu Trp Leu Lys Asn Gly Gly NMeala Ser Ser Gly Ala NMeAla NmeAla-NH₂ |
| 85 | His Gly Glu Gly Thr Phe Thr Ser Asp Leu Ser Lys Gln Met Glu Glu Glu Ala Val Arg Leu |
| | Phe Ile Glu Trp Leu Lys Asn Gly Gly homoPro Ser Ser Gly Ala homoPro homoPro-NH₂ |
| 86 | His Gly Glu Gly Thr Phe Thr Ser Asp Leu Ser Lys Gln Met Glu Glu Glu Ala Val Arg Leu |
| | Phe Ile Glu Trp Leu Lys Asn Gly Gly homoPro Ser Ser Gly Ala homoPro-NH₂ |
| 87 | Arg Gly Glu Gly Thr Phe Thr Ser Asp Leu Ser Lys Gln Met Glu Glu Glu Ala Val Arg Leu |
| | Phe Ile Glu Trp Leu Lys Asn Gly Gly Pro Ser Ser Gly Ala-NH₂ |
| 88 | His Gly Asp Gly Thr Phe Thr Ser Asp Leu Set Lys Gln Met Glu Glu Glu Ala Val Arg Leu |
| | Phe Ile Glu Trp Leu Lys Asn Gly Gly-NH₂ |
| 89 | His Gly Glu Gly Thr NaphthylAla Thr Ser Asp Leu Ser Lys Gln Leu Glu Glu Glu Ala Val |
| | Arg Leu Phe Ile Glu Phe Leu Lys Asn-NH₂ |
| 90 | His Gly Glu Gly Thr Phe Ser Ser Asp Leu Ser Lys Gln Met Glu Glu Glu Ala Val Arg Leu |
| | Phe Ile Glu Trp Leu Lys Asn-NH₂ |
| 91 | His Gly Glu Gly Thr Phe Ser Thr Asp Leu Ser Lys Gln Met Glu Glu Glu Ala Val Arg Leu |
| | Phe Ile Glu Trp Leu Lys Asn-NH₂ |
| 92 | His Gly Glu Gly Thr Phe Thr Ser Glu Leu Ser Lys Gln Met Ala Glu Glu Ala Val Arg Leu |
| | Phe Ile Glu Trp Leu Lys Asn-NH₂ |
| 93 | His Gly Glu Gly Thr Phe Thr Ser Asp pentylGly Ser Lys Gln Leu Glu Glu Glu Ala Val Arg |
| | Leu Phe Ile Glu Phe Leu Lys Asn-NH₂ |
| 94 | His Gly Glu Gly Thr Phe Thr Ser Asp Leu Ser Lys Gln Leu Glu Glu Glu Ala Val Arg Leu |
| | NahthylAla Ile Glu Phe Leu Lys Asn-NH₂ |
| 95 | His Gly Glu Gly Thr Phe Thr Ser Asp Leu Ser Lys Gln Met Glu Glu Glu Ala Val Arg Leu |
| | Phe tButylGly Glu Trp Leu Lys Asn-NH₂ |
| 96 | His Gly Glu Gly Thr Phe Thr Ser Asp Leu Ser Lys Gln Leu Glu Glu Glu Ala Val Arg Leu |
| | Phe Ile Asp Phe Leu Lys Asn-NH₂ |
| 97 | His Gly Glu Gly Thr Phe Thr Ser Asp Ala Ser Lys Gln Leu Glu Glu Glu Ala Val Arg Leu |
| | Phe Ile Glu Phe Leu Lys Asn Gly Gly Pro Ser Ser-NH₂ |
| 98 | His Gly Glu Gly Thr Phe Thr Ser Asp Ala Ser Lys Gln Met Glu Glu Glu Ala Val Arg Leu |
| | Phe Ile Glu Trp Leu Lys Asn Gly-NH₂ |
| 99 | His Gly Glu Gly Thr Phe Thr Ser Asp Ala Ser Lys Gln Met Glu Glu Glu Ala Val Arg Leu |
| | Phe Ile Glu Trp Leu Lys Asn Gly Gly homoPro Ser Ser Gly Ala homoPro homoPro-NH₂ |
| 100 | Ala Gly Glu Gly Thr Phe Thr Ser Asp Leu Ser Lys Gln Leu Glu Glu Glu Ala Val Arg Leu |
| | Phe Ile Glu Phe Leu Lys Asn-NH₂ |
| 101 | His Gly Ala Gly Thr Phe Thr Ser Asp Leu Ser Lys Gln Leu Glu Glu Glu Ala Val Arg Leu |
| | Phe Ile Glu Phe Leu Lys Asn-NH₂ |
| 102 | His Gly Glu Ala Thr Phe Thr Ser Asp Leu Ser Lys Gln Leu Glu Glu Glu Ala Val Arg Leu |
| | Phe Ile Glu Phe Leu Lys Asn-NH₂ |
| 103 | His Gly Glu Gly Thr Phe Thr Ser Ala Leu Ser Lys Gln Leu Glu Glu Glu Ala Val Arg Leu |
| | Phe Ile Glu Phe Leu Lys Asn-NH₂ |
| 104 | Ala Gly Glu Gly Thr Phe Thr Ser Asp Leu Ser Lys Gln Met Glu Glu Glu Ala Val Arg Leu |
| | Phe Ile Glu Trp Leu Lys Asn-NH₂ |
| 105 | His Gly Ala Gly Thr Phe Thr Ser Asp Leu Ser Lys Gln Met Glu Glu Glu Ala Val Arg Leu |
| | Phe Ile Glu Trp Leu Lys Asn-NH₂ |
| 106 | His Gly Glu Ala Thr Phe Thr Ser Asp Leu Ser Lys Gln Met Glu Glu Glu Ala Val Arg Leu |
| | Phe Ile Glu Trp Leu Lys Asn-NH₂ |
| 107 | His Gly Glu Gly Thr Phe Thr Ser Ala Leu Ser Lys Gln Met Glu Glu Glu Ala Val Arg Leu |
| | Phe Ile Glu Trp Leu Lys Asn-NH₂ |
| 108 | His Gly Glu Gly Thr Phe Thr Ser Asp Ala Ser Lys Gln Met Glu Glu Glu Ala Val Arg Leu |
| | Phe Ile Glu Trp Leu Lys Asn-NH₂ |
| 109 | Ala Ala Glu Gly Thr Phe Thr Ser Asp Leu Ser Lys Gln Met Glu Glu Glu Ala Val Arg Leu |
| | Phe Ile Glu Trp Leu Lys Asn-NH₂ |
| 110 | Ala Ala Glu Gly Thr Phe Thr Set Asp Leu Ser Lys Gln Leu Glu Glu Glu Ala Val Arg Leu |
| | Phe Ile Glu Phe Leu Lys Asn-NH₂ |
| 111 | Ala Gly Asp Gly Thr Phe Thr Ser Asp Leu Ser Lys Gln Met Glu Glu Glu Ala Val Arg Leu |
| | Phe Ile Glu Trp Leu Lys Asn-NH₂ |
| 112 | Ala Gly Asp Gly Thr Phe Thr Ser Asp Leu Ser Lys Gln Leu Glu Glu Glu Ala Val Arg Leu |
| | Phe Ile Glu Phe Leu Lys Asn-NH₂ |
| 113 | Ala Gly Asp Gly Ala Phe Thr Ser Asp Leu Ser Lys Gln Met Glu Glu Glu Ala Val Arg Leu |
| | Phe Ile Glu Trp Leu Lys Asn-NH₂ |
| 114 | Ala Gly Asp Gly Ala Phe Thr Ser Asp Leu Ser Lys Gln Leu Glu Glu Glu Ala Val Arg Leu |
| | Phe Ile Glu Phe Leu Lys Asn-NH₂ |
| 115 | Ala Gly Asp Gly Thr NaphthylAla Thr Ser Asp Leu Ser Lys Gln Met Glu Glu Glu Ala Val |
| | Arg Leu Phe Ile Glu Trp Leu Lys Asn-NH₂ |
| 116 | Ala Gly Asp Gly Thr NaphthylAla Thr Set Asp Leu Ser Lys Gln Leu Glu Glu Glu Ala Val |
| | Arg Leu Phe Ile Glu Phe Leu Lys Asn-NH₂ |
| 117 | Ala Gly Asp Gly Thr Phe Ser Ser Asp Leu Ser Lys Gln Met Glu Glu Glu Ala Val Arg Leu |
| | Phe Ile Glu Trp Leu Lys Asn-NH₂ |
| 118 | Ala Gly Asp Gly Thr Phe Ser Ser Asp Len Ser Lys Gln Leu Glu Glu Glu Ala Val Arg Leu |
| | Phe Ile Glu Phe Leu Lys Asn-NH₂ |
| 119 | Ala Gly Asp Gly Thr Phe Thr Ala Asp Leu Ser Lys Gln Met Glu Glu Glu Ala Val Arg Leu |
| | Phe Ile Glu Trp Leu Lys Asn-NH₂ |
| 120 | Ala Gly Asp Gly Thr Phe Thr Ala Asp Leu Ser Lys Gln Leu Glu Gln Glu Ala Val Arg Leu |
| | Phe Ile Gln Phe Leu Lys Asn-NH₂ |
| 121 | Ala Gly Asp Gly Thr Phe Thr Ser Ala Leu Ser Lys Gln Met Glu Gln Glu Ala Val Arg Leu |
| | Phe Ile Gln Trp Leu Lys Asn-NH₂ |
| 122 | Ala Gly Asp Gly Thr Phe Thr Ser Ala Leu Ser Lys Gln Leu Glu Gln Glu Ala Val Arg Leu |
| | Phe Ile Glu Phe Leu Lys Asn-NH₂ |
| 123 | Ala Gly Asp Gly Thr Phe Thr Ser Gln Leu Ser Lye Gln Met Glu Glu Gln Ala Val Arg Leu |
| | Phe Ile Glu Trp Leu Lys Asn-NH₂ |
| 124 | Ala Gly Asp Gly Thr Phe Thr Ser Gln Leu Ser Lys Gln Len Glu Gln Glu Ala Val Arg Leu |
| | Phe Ile Glu Phe Len Lys Asn-NH₂ |
| 125 | Ala Gly Asp Gly Thr Phe Thr Ser Asp Ala Ser Lys Gln Met Gln Glu Gln Ala Val Arg Leu |
| | Phe Ile Gln Trp Leu Lys Asn-NH₂ |
| 126 | Ala Gly Asp Gly Thr Phe Thr Ser Asp Ala Ser Lys Gln Leu Gln Gln Gln Ala Val Arg Leu |
| | Phe Ile Gln Phe Leu Lys Asn-NH₂ |
| 127 | Ala Gly Asp Gly Thr Phe Thr Ser Asp pentylGly Ser Lys Gln Met Glu Glu Glu Ala Val A |
| | Leu Phe Ile Glu Trp Leu Lys Asn-NH₂ |
| 128 | Ala Gly Asp Gly Thr Phe Thr Ser Asp pentylGly Ser Lys Gln Leu Gln Gln Glu Ala Val Arg |
| | Len Phe Ile Glu Phe Leu Lys Asn-NH₂ |
| 129 | Ala Gly Asp Gly Thr Phe Thr Ser Asp Len Ala Lys Gln Met Gln Glu Glu Ala Arg Len |
| | Phe Ile Glu Trp Leu Lys Asn-NH₂ |
| 130 | Na Ala Gly Asp Gly Thr Phe Thr Ser Asp Leu Ala Lys Gln Leu Glu Glu Glu Ala Val |
| | Phe Ile Glu Phe Leu Lys Asn-NH₂ |
| 131 | Ala Gly Asp Gly Thr Phe Thr Ser Asp Leu Ser Ala Gln Met Glu Glu Glu Ala Val Arg Leu |
| | Phe Ile Glu Trp Leu Lys Asn-NH₂ |
| 132 | Ala Gly Asp Gly Thr Phe Thr Ser Asp Leu Ser Ala Gln Leu Glu Glu Glu Ala Val Arg Leu |
| | Phe Ile Glu Phe Leu Lys Asn-NH₂ |
| 133 | Ala Gly Asp Gly Thr Phe Thr Ser Asp Leu Ser Lys Ala Met Glu Glu Glu Ala Val Arg Leu |
| | Phe Ile Glu Trp Leu Lys Asn-NH₂ |
| 134 | Ala Gly Asp Gly Thr Phe Thr Ser Asp Leu Ser Lys Ala Leu Glu Glu Glu Ala Val Arg Leu |
| | Phe Ile Glu Phe Leu Lys Asn-NH₂ |
| 135 | Ala Gly Asp Gly Thr Phe Thr Set Asp Leu Ser Lys Gln Ala Glu Glu Glu Ala Val Arg Leu |
| | Phe Ile Glu Trp Leu Lys Asn-NH₂ |
| 136 | Ala Gly Asp Gly Thr Phe Thr Ser Asp Leu Ser Lys Gln Ala Glu Glu Glu Ala Val Arg Leu |
| | Phe Ile Glu Phe Leu Lys Asn-NH₂ |
| 137 | Ala Gly Asp Gly Thr Phe Thr Ser Asp Leu Ser Lys Gln pentylGly Glu Glu Glu Ala Val Arg |
| | Leu Phe Ile Glu Trp Leu Lys Asn-NH₂ |
| 138 | Ala Gly Asp Gly Thr Phe Thr Ser Asp Leu Ser Lys Gln pentylGly Glu Glu Glu Ala Val Arg |
| | Leu Phe Ile Glu Phe Leu Lys Asn-NH₂ |
| 139 | Ala Gly Asp Gly Thr Phe Thr Ser Asp Leu Ser Lys Gln Met Ala Glu Glu Ala Val Arg Leu |
| | Phe Ile Glu Trp Leu Lys Asn-NH₂ |
| 140 | Ala Gly Asp Gly Thr Phe Thr Ser Asp Leu Ser Lys Gln Leu Ala Glu Glu Ala Val Arg Leu |
| | Phe Ile Glu Phe Leu Lys Asn-NH₂ |
| 141 | Ala Gly Asp Gly Thr Phe Thr Set Asp Leu Ser Lys Gln Met Glu Ala Glu Ala Val Arg Leu |
| | Phe Ile Glu Trp Leu Lys Asn-NH₂ |
| 142 | Ala Gly Asp Gly Thr Phe Thr Ser Asp Leu Ser Lys Gln Leu Glu Ala Glu Ala Val Arg Leu |
| | Phe Ile Glu Phe Leu Lys Asn-NH₂ |
| 143 | Ala Gly Asp Gly Thr Phe Thr Ser Asp Leu Ser Lys Gln Met Glu Glu Ala Ala Val Arg Leu |
| | Phe Ile Glu Trp Leu Lys Asn-NH₂ |
| 144 | Ala Gly Asp Gly Thr Phe Thr Ser Asp Leu Ser Lys Gln Leu Glu Glu Ala Ala Val Arg Leu |
| | Phe Ile Glu Phe Leu Lys Asn-NH₂ |
| 145 | Ala Gly Asp Gly Thr Phe Thr Ser Asp Leu Ser Lys Gln Met Glu Glu Glu Ala Ala Arg Leu |
| | Phe Ile Glu Trp Leu Lys Asn-NH₂ |
| 146 | Ala Gly Asp Gly Thr Phe Thr Ser Asp Leu Ser Lye Gln Leu Glu Glu Glu Ala Ala Arg Leu |
| | Phe Ile Glu Phe Leu Lys Asn-NH₂ |
| 147 | Ala Gly Asp Gly Thr Phe Thr Ser Asp Leu Ser Lys Gln Met Glu Glu Glu Ala Val Ala Leu |
| | Phe Ile Glu Trp Leu Lys Asn-NH₂ |
| 148 | Ala Gly Asp Gly Thr Phe Thr Ser Asp Leu Ser Lys Gln Leu Glu Glu Glu Ala Val Ala Leu |
| | Phe Ile Glu Phe Leu Lys Asn-NH₂ |
| 149 | Ala Gly Asp Gly Thr Phe Thr Ser Asp Leu Ser Lys Gln Met Glu Glu Glu Ala Val Arg Ala |
| | Phe Ile Glu Trp Leu Lys Asn-NH₂ |
| 150 | Ala Gly Asp Gly Thr Phe Thr Ser Asp Leu Ser Lys Gln Leu Glu Glu Glu Ala Val Arg Ala |
| | Phe Ile Glu Phe Leu Lys Asn-NH₂ |
| 151 | Ala Gly Asp Gly Thr Phe Thr Ser Asp Leu Ser Lys Gln Met Glu Glu Glu Ala Val Arg Leu |
| | Naphthylala Ile Glu Trp Leu Lys Asn-NH₂ |
| 152 | Ala Gly Asp Gly Thr Phe Thr Ser Asp Leu Ser Lys Gln Leu Glu Glu Glu Ala Val Arg Leu |
| | Naphthylala Ile Glu Phe Leu Lys Asn-NH₂ |
| 153 | Ala Gly Asp Gly Thr Phe Thr Ser Asp Leu Ser Lys Gln Met Glu Glu Glu Ala Val Arg Leu |
| | Phe Val Glu Trp Leu Lys Asn-NH₂ |
| 154 | Ala Gly Asp Gly Thr Phe Thr Ser Asp Leu Ser Lys Gln Leu Glu Glu Glu Ala Val Arg Leu |
| | Phe Val Glu Phe Leu Lys Asn-NH₂ |
| 155 | Ala Gly Asp Gly Thr Phe Thr Ser Asp Leu Ser Lys Gln Met Glu Glu Glu Ala Val Arg Leu |
| | Phe tButylgly Glu Trp Leu Lys Asn-NH₂ |
| 156 | Ala Gly Asp Gly Thr Phe Thr Set Asp Leu Ser Lys Gln Leu Glu Glu Glu Ala Val Arg Leu |
| | Phe tButylgly Glu Phe Leu Lys Asn-NH₂ |
| 157 | Ala Gly Asp Gly Thr Phe Thr Ser Asp Leu Ser Lys Gln Met Glu Glu Glu Ala Val Arg Leu |
| | Phe Ile Asp Trp Leu Lys Asn-NH₂ |
| 158 | Ala Gly Asp Gly Thr Phe Thr Ser Asp Leu Ser Lys Gln Leu Glu Glu Glu Ala Val Arg Leu |
| | Phe Ile Asp Phe Leu Lys Asn-NH₂ |
| 159 | Ala Gly Asp Gly Thr Phe Thr Set Asp Leu Ser Lys Gln Met Glu Glu Glu Ala Val Arg Leu |
| | Phe Ile Glu Ala Leu Lys Asn-NH₂ |
| 160 | Ala Gly Asp Gly Thr Phe Thr Ser Asp Leu Ser Lys Gln Leu Glu Glu Glu Ala Val Arg Leu |
| | Phe Ile Glu Ala Leu Lys Asn-NH₂ |
| 161 | Ala Gly Asp Gly Thr Phe Thr Ser Asp Leu Ser Lys Gln Met Glu Glu Glu Ala Val Arg Leu |
| | Phe Ile Glu Trp Ala Lys Asn-NH₂ |
| 162 | Ala Gly Asp Gly Thr Phe Thr Ser Asp Leu Ser Lys Gln Leu Glu Glu Glu Ala Val Arg Leu |
| | Phe Ile Glu Phe Ala Lys Asn-NH₂ |
| 163 | Ala Gly Asp Gly Thr Phe Thr Ser Asp Leu Ser Lys Gln Met Glu Glu Glu Ala Val Arg Leu |
| | Phe Ile Glu Trp Leu Ala Asn-NH₂ |
| 164 | Ala Gly Asp Gly Thr Phe Thr Ser Asp Leu Ser Lys Gln Leu Glu Glu Glu Ala Val Arg Leu |
| | Phe Ile Glu Phe Leu Ala Asn-NH₂ |
| 165 | Ala Gly Asp Gly Thr Phe Thr Ser Asp Leu Ser Lys Gln Met Glu Glu Glu Ala Val Arg Leu |
| | Phe Ile Glu Trp Leu Lys Ala-NH₂ |
| 166 | Ala Gly Asp Gly Thr Phe Thr Ser Asp Leu Ser Lys Gln Leu Glu Glu Glu Ala Val Arg Leu |
| | Phe Ile Glu Phe Leu Lys Ala-NH₂ |
| 167 | Ala Gly Glu Gly Thr Phe Thr Ser Asp Leu Ser Lys Gln Met Glu Glu Glu Ala Val Arg Leu |
| | Phe Ile Glu Trp Leu Lys Asn Gly Gly Pro Ser Ser Gly Ala Pro Pro Pro-NH₂ |
| 168 | His Gly Ala Gly Thr Phe Thr Ser Asp Leu Ser Lys Gln Leu Glu Glu Glu Ala Val Arg Leu |
| | Phe Ile Glu Phe Leu Lys Asn Gly Gly Pro Ser Ser Gly Ala Pro Pro Pro-NH₂ |
| 169 | His Gly Glu Ala Thr Phe Thr Ser Asp Leu Ser Lys Gln Met Glu Glu Glu Ala Val Arg Leu |
| | Phe Ile Glu Trp Leu Lys Asn Gly Gly Pro Ser Ser Gly Ala Pro Pro-NH₂ |
| 170 | His Gly Glu Gly Thr Phe Thr Ser Ala Leu Ser Lys Gln Met Glu Glu Glu Ala Val Arg Leu |
| | Phe Ile Glu Trp Leu Lys Asn Gly Gly Pro Ser Ser Gly Ala Pro-NH₂ |
| 171 | Ala Gly Glu Gly Thr Phe Thr Ser Asp Ala Ser Lys Gln Leu Glu Glu Glu Ala Val Arg Leu |
| | Phe Ile Glu Phe Leu Lys Asn Gly Gly Pro Ser Ser Gly Ala Pro-NH₂ |
| 172 | Ala Gly Glu Gly Thr Phe Thr Ser Asp Leu Ser Lys Gln Met Glu Glu Glu Ala Val Arg Leu |
| | Phe Ile Glu Trp Leu Lys Asn Gly Gly Pro Ser Ser Gly Ala-NH₂ |
| 173 | His Gly Ala Gly Thr Phe Thr Ser Asp Leu Ser Lys Gln Leu Glu Glu Glu Ala Val Arg Leu |
| | Phe Ile Glu Phe Leu Lys Asn Gly Gly Pro Ser Ser Gly Ala-NH₂ |
| 174 | His Gly Glu Ala Thr Phe Thr Ser Asp Leu Ser Lys Gln Met Glu Glu Glu Ala Val Arg Leu |
| | Phe Ile Glu Trp Leu Lys Asn Gly Gly Pro Ser Ser Gly-NH₂ |
| 175 | His Gly Glu Gly Thr Phe Thr Set Ala Leu Ser Lys Gln Met Glu Glu Glu Ala Val Arg Leu |
| | Phe Ile Glu Trp Leu Lys Asn Gly Gly Pro Ser Ser-NH₂ |
| 176 | Ala Gly Glu Gly Thr Phe Thr Ser Asp Leu Ser Lys Gln Met Glu Glu Glu Ala Val Arg Leu |
| | Phe Ile Glu Trp Leu Lys Asn Glu Gly Pro Ser-NH₂ |
| 177 | His Gly Ala Gly Thr Phe Thr Ser Asp Leu Ser Lys Gln Leu Glu Glu Glu Ala Val Arg Leu |
| | Phe Ile Glu Phe Leu Lys Asn Gly Gly Pro Ser-NH₂ |
| 178 | His Gly Glu Ala Thr Phe Thr Ser Asp Leu Ser Lys Gln Met Glu Glu Glu Ala Val Arg Leu |
| | Phe Ile Glu Trp Leu Lys Asn Gly Gly Pro-NH₂, |
| 179 | His Gly Glu Gly Thr Phe Thr Ser Ala Leu Ser Lys Gln Leu Glu Glu Glu Ala Val Arg Leu |
| | Phe Ile Glu Phe Leu Lys Asn Gly Gly-NH₂ |
| 180 | Ala Gly Glu Gly Thr Phe Thr Ser Asp Leu Ser Lys Gln Leu Glu Glu Glu Ala Val Arg Leu |
| | Phe Ile Glu Phe Leu Lys Asn Gly-NH₂ |
| 181 | His Gly Ala Gly Thr Phe Thr Ser Asp Leu Ser Lys Gln Met Glu Glu Glu Ala Val Arg Leu |
| | Phe Ile Glu Trp Leu Lys Asn Gly Gly thioPro Ser Ser Gly Ala thioPro thioPro thioPro- |
| | NH₂ |
| 182 | His Gly Glu Ala Thr Phe Thr Ser Asp Leu Ser Lys Gln Met Glu Glu Glu Ala Val Arg Leu |
| | Phe Ile Glu Trp Leu Lys Asn Gly Gly Pro Ser Ser Gly Ala thioPro thioPro thioPro-NH₂ |
| 183 | His Gly Glu Gly Thr Phe Thr Ser Ala Leu Ser Lys Gln Met Glu Glu Glu Ala Val Arg Leu |
| | Phe Ile Glu Trp Leu Lys Asn Gly Gly NMeala Ser Ser Gly Ala NMeala NMeala-NH₂ |
| 184 | Ala Gly Glu Gly Thr Phe Thr Ser Asp Leu Ser Lys Gln Met Glu Glu Glu Ala Val Arg Leu |
| | Phe Ile Glu Trp Leu Lys Asn Gly Gly homoPro Ser Ser Gly Ala homoPro-NH₂ |
| 185 | His Gly Ala Gly Thr Phe Thr Ser Asp Leu Ser Lys Gln Met Glu Glu Glu Ala Val Arg Leu |
| | Phe Ile Glu Trp Leu Lys Asn Gly Gly Pro Ser Ser Gly Ala-NH₂ |
| 186 | His Gly Asp Ala Thr Phe Thr Ser Asp Leu Ser Lys Gln Met Glu Glu Glu Ala Val Arg Leu |
| | Phe Ile Glu Trp Leu Lys Asn Gly Gly-NH₂ |
| 187 | Ala Gly Glu Gly Thr Phe Thr Ser Asp Leu Ser Lys Gln Met Glu Glu Glu Ala Val Arg Leu |
| | Phe Ile Glu Trp Leu Lys Asn Gly Gly Pro Ser Ser Gly Ala Pro Pro Pro Ser-NH₂ |
| 188 | Ala Gly Ala Gly Thr Phe Thr Ser Asp Leu Ser Lys Gln Leu Glu Glu Glu Ala Val Arg Leu |
| | Phe Ile Glu Phe Leu Lys Asn Gly Gly Pro Ser Ser Gly Ala Pro Pro Pro Ser-NH₂ |

In one embodiment, the bioactive peptide or protein of the compositions described herein comprise PYY peptides, PYY peptide analogs and PYY derivatives, such as PYY₃₋₃₆. Additional PYY peptides that can be used in the compositions disclosed herein include any bioactive PYY peptide, PYY analog or PYY derivative known in the art such as those as described in International Patent Application Publication Nos. WO 02/47712 and WO 03/26591; and US Patent Application Publication No. 2002-141985, all of which are herein incorporated by reference in their entireties and in particular the PYY-related sequences disclosed therein. By “PYY” or “PYY peptide” is meant a Peptide YY polypeptide obtained or derived from any species. Thus, the term “PYY” includes the 36 amino acid full length human as well as species variations of PYY, including, but not limited to, murine, hamster, chicken, bovine, rat and dog PYY. Particular examples of PYY peptides, PYY analogs and PYY derivatives that can be used in the compositions disclosed herein, include, but are not limited to those described in Table 2. Also included are other Y receptor family peptide agonists, particularly Y2, Y5, and putative Y7 receptor agonists and derivatives thereof. In one embodiment, the bioactive peptide is PYY₃₋₃₆. PYY peptides are known to have activity in food intake, gastric emptying, pancreatic secretion and weight loss.

TABLE 2
PYY Peptides, Analogs and Derivatives

| SEQ ID | |
|---|---|
| NO | Sequence |
| 189 | Ala Pro Leu Glu Pro Val Tyr Pro Gly Asp Asn Ala Thr Pro Glu Gln Met |
| | Ala Gln Tyr Ala Ala Asp Leu Arg Arg Tyr Ile Asn Met Leu Thr Arg Pro |
| | Arg Tyr |
| 190 | Tyr Pro Ile Lys Pro Glu Ala Pro Gly Glu Asp Ala Ser Pro Glu Glu Leu Asn |
| | Arg Tyr Tyr Ala Ser Leu Arg His Tyr Leu Asn Leu Val Thr Arg Gln Arg |
| | Tyr |
| 191 | Ile Lys Pro Glu Ala Pro Gly Glu Asp Ala Ser Pro Glu Glu Leu Asn Arg Tyr |
| | Tyr Ala Ser Leu Arg His Tyr Leu Asn Leu Val Thr Arg Gln Arg Tyr |
| 192 | Tyr Pro Ser Lys Pro Asp Asn Pro Gly Glu Asp Ala Pro Ala Glu Asp Met |
| | Ala Arg Tyr Tyr Ser Ala Leu Arg His Tyr Ile Asn Leu Ile Thr Arg Gln Arg |
| | Tyr |
| 193 | Ser Lys Pro Asp Asn Pro Gly Glu Asp Ala Pro Ala Glu Asp Met Ala Arg |
| | Tyr Tyr Ser Ala Leu Arg His Tyr Ile Asn Leu Ile Thr Arg Gln Arg Tyr |
| 194 | Ala Ser Leu Arg His Tyr Leu Asn Leu Val Thr Arg Gln Arg Tyr |

In additional embodiments, the bioactive peptide or protein of the compositions disclosed herein comprise GLP-1, GLP-1 analogs and GLP-1 derivatives such as GLP-1 (7-37), GLP-1 (7-36)NH₂, Gly⁸ GLP-1 (7-37), Ser³⁴ GLP-1 (7-37) Val⁸ GLP-1 (7-37) and Val⁸ Glu²² GLP-1 (7-37). Any bioactive GLP-1, GLP-1 analog or GLP-1 derivative known in the art can be used in the present compositions, including, but not limited to those described in International Patent Application Publications Nos. WO 01/98331, WO 02/48192; US Patent Application Nos. 2003-220243 and 2004-053819; and U.S. Pat. Nos. 5,981,488, 5,574,008, 5,512,549, and 5,705,483, all of which are herein incorporated by reference in their entireties and in particular the GLP-1-related sequences described therein. Examples of GLP-1 peptides that are suitable for use in the compositions disclosed herein are those described in US Patent Application 2003-220243 by the following formulas:

Formula IV
(SEQ ID No. 244)
His-Xaa₈-Glu-Gly-Xaa₁₁-Xaa₁₂-Thr-Ser-Asp-Xaa₁₆-Ser-Ser-Tyr-Leu-Glu-Xaa₂₂-
Xaa₂₃-Xaa₂₄-Ala-Xaa₂₆-Xaa₂₇-Phe-Ile-Ala-Xaa₃₁-Leu-Xaa₃₃-Xaa₃₄-Xaa₃₅-Xaa₃₆-R

where:

### Xaa₈ is Gly, Ala, Val, Leu, Ile, Ser, or Thr;

### Xaa₁₁ is Asp, Glu, Arg, Thr, Ala, Lys, or His;

### Xaa₁₂ is His, Trp, Phe, or Tyr,

### Xaa₁₆ is Leu, Ser, Thr, Trp, His, Phe, Asp, Val, Glu, or Ala;

### Xaa₂₂ is Gly, Asp, Glu, Gin, Asn, Lys, Arg, Cys, or Cysteic Acid;

### Xaa₂₃ is His, Asp, Lys, Glu, or Gin;

### Xaa₂₄ is Glu, His, Ala, or Lys;

### Xaa₂₆ is Asp, Lys, Glu, or His;

### Xaa₂₇ is Ala, Glu, His, Phe, Tyr, Trp, Arg, or Lys;

### Xaa₃₁ is Ala, Glu, Asp, Ser, or His;

### Xaa₃₃ is Asp, Arg, Val, Lys, Ala, Gly, or Glu;

### Xaa₃₄ is Glu, Lys, or Asp;

### Xaa₃₅ is Thr, Ser, Lys, Arg, Trp, Tyr, Phe, Asp, Gly, Pro, His, or Glu;

### Xaa₃₆ is Arg, Glu, or His; and

R is: Lys, Arg, Thr, Ser, Glu, Asp, Trp, Tyr, Phe, His, —NH₂, Gly, Gly-Pro, or Gly-Pro-NH₂, or is deleted.

Formula V
(SEQ ID No. 245)
His-Xaa₈-Glu-Gly-Thr-Xaa₁₂-Thr-Ser-Asp-Xaa₁₆-Ser-Ser-Tyr-Leu-Glu-Xaa₂₂-Xaa₂₃-
Ala-Ala-Xaa₂₆-Glu-Phe-Ile-Xaa₃₀-Trp-Leu-Val-Lys-Xaa₃₅-Arg-R

where:
Xaa₈ is Gly, Ala, Val, Leu, ile, Ser, or Thr;

### Xaa₁₂ is His, Trp, Phe, or Tyr,

### Xaa₁₆ is Leu, Ser, Thr, Trp, His, Phe, Asp, Val, Glu, or Ala;

### Xaa₂₂ is Gly, Asp, Glu, Gin, Asn, Lys, Arg, Cys, or Cysteic Acid (3-Sulfoalanine);

### Xaa₂₃ is His, Asp, Lys, Glu, or Gln;

### Xaa₂₆ is: Asp, Lys, Glu, or His;

### Xaa₃₀ is Ala, Glu, Asp, Ser, or His;

Xaa₃₅ is Thr, Ser, Lys, Arg, Trp, Tyr, Phe, Asp, Gly. Pro, His, or Glu; and
R is: Lys, Arg, Thr, Ser, Glu, Asp, Trp, Tyr, Phe, His, —NH₂, Gly, Gly-Pro, or Gly-Pro-NH₂, or is deleted.

Formula VI
(SEQ ID No. 246)
His-Xaa₈-Glu-Gly-Thr-Phe-Thr-Ser-Asp-Val-Ser-Ser-Tyr-Leu-Glu-Xaa₂₂-Xaa₂₃-Ala-
Ala-Lys-Xaa₂₇-Phe-Ile-Xaa₃₀-Trp-Leu-Val-Lys-Gly-Arg-R

where:

### Xaa₈ is Gly, Ala, Val, Leu, Ile, Set, or Thr,

### Xaa₂₂ is Gly, Asp, Glu, Gin, Asn, Lys, Arg, Cys, or Cysteic Acid (3-Sulfoalanine);

### Xaa₂₃ is His, Asp, Lys, Glu, or Gin;

### Xaa₂₇ is Ala, Glu, His, Phe, Tyr, Trp, Arg, or Lys

### Xaa₃₀ is Ala, Glu, Asp, Ser, or His; and

R is: Lys, Arg, Thr, Ser, Glu, Asp, Trp, Tyr, Phe, His, —NH₂, Gly, Gly-Pro, or Gly-Pro-NH₂, or is deleted.

Formula VII
(SEQ ID No. 247)
Xaa₇-Xaa₈-Glu-Gly-Thr-Phe-Thr-Ser-Asp-Val-Ser-Ser-Tyr-Leu-Glu-Xaa₂₂-Gln-Ala-
Ala-Lys-Glu-Phe-Ile-Ala-Trp-Leu-Val-Lys-Gly-Arg-R

where:
Xaa₇ is L-histidine, D-histidine, desamino-histidine, 2amino-histidine, β-hydroxy-histidine, homohistidine, α-fluoromethyl-histidine or α-methyl-histidine;
Xaa₈ is glycine, alanine, valine, leucine, isoleucine, serine or threonine;
Xaa₂₂ is aspartic acid, glutamic acid, glutamine, asparagine, lysine, arginine, cysteine, or cysteic acid; and

### R is —NH₂ or Gly(OH).

Particular, but non-limiting examples of GLP1 peptides that can be use in the present compositions can be found in Table 3

TABLE 3
GLP-1 Peptides, Analogs and Derivatives

| SEQ | |
|---|---|
| ID | |
| NO | Sequence |
| 195 | His Val Glu Gly Thr Phe Thr Ser Asp Val Ser Ser Tyr Leu Glu Glu Gln Ala Ala Lys Glu Phe |
| | Ile Ala Trp Leu Val LysGly Arg Gly |
| 196 | His Val Glu Gly Thr Phe Thr Ser Asp Val Set Ser Tyr Leu Glu Asp Gln Ala Ala Lys Glu Phe |
| | Ile Ala Trp Leu Val Lys Gly Arg Gly |
| 197 | His Val Glu Gly Thr Phe Thr Ser Asp Val Ser Ser Tyr Leu Glu Arg Gln Ala Ala Lys Glu Phe |
| | Ile Ala Trp Leu Val Lys Gly Arg Gly |
| 198 | His Val Glu Gly Thr Phe Thr Ser Asp Val Ser Ser Tyr Leu Glu Lys Gln Ala Ala Lys Glu Phe |
| | Ile Ala Trp Leu Val Lys Gly Arg Gly |
| 199 | His Gly Glu Gly Thr Phe Thr Ser Asp Val Ser Ser Tyr Leu Glu Glu Gln Ala Ala Lys Glu Phe |
| | Ile Ala Trp Leu Val Lys Gly Arg Gly |
| 200 | His Gly Glu Gly Thr Phe Thr Ser Asp Val Ser Ser Tyr Leu Glu Asp Gln Ala Ala Lys Glu Phe |
| | Ile Ala Trp Leu Val Lys Gly Arg Gly |
| 201 | His Gly Glu Gly Thr Phe Thr Ser Asp Val Ser Ser Tyr Leu Glu Arg Gln Ala Ala Lys Glu Phe |
| | Ile Ala Trp Leu Val Lys Gly Arg Gly |
| 202 | His Gly Glu Gly Thr Phe Thr Ser Asp Val Ser Ser Tyr Leu Glu Lys Gln Ala Ala Lys Glu Phe |
| | Ile Ala Trp Leu Val Lys Gly Arg Gly |
| 203 | His Val Glu Gly Thr Phe Thr Set Asp Val Ser Ser Tyr Leu Glu Gly Gln Ala Ala Lys Glu Phe |
| | Ile Glu Trp Leu Val Lys Gly Arg Gly |
| 204 | His Gly Glu Gly Thr Phe Thr Ser Asp Val Ser Ser Tyr Leu Glu Gly Gln Ala Ala Lys Glu Phe |
| | Ile Glu Trp Leu Val Lys Gly Arg Gly |
| 205 | His Val Glu Gly Thr Phe Thr See Asp Val Ser Ser Tyr Leu Glu Gly Gln Ala Ala Lys Glu Phe |
| | Ile Ala Trp Leu Val Lys Gly Arg His |
| 206 | His Gly Glu Gly Thr Phe Thr Ser Asp Val Ser Ser Tyr Leu Glu Gly Gln Ala Ala Lys Glu Phe |
| | Ile Ala Trp Leu Val Lys Gly Arg His |
| 207 | His Val Glu Gly Thr Phe Thr Ser Asp Val Ser Ser Tyr Leu Glu Glu Gln Ala Ala Lys Ala Phe |
| | Ile Ala Trp Leu Val Lys Gly Arg His |
| 208 | His Val Glu Gly Thr Phe Thr Ser Asp Val Ser Ser Tyr Leu Glu Lys Glu Ala Ala Lys Gin Phe |
| | Ile Ala Trp Leu Val Lys Gly Arg His |
| 209 | His Ala Glu Gly Thr Phe Thr Ser Asp Val Ser Ser Tyr Leu Glu Gly Gln Ala Ala Lys Glu Phe |
| | Ile Ala Trp Leu Val Lys Gly Arg Gly |
| 210 | His Ala Glu Gly Thr Phe Thr Ser Asp Val Ser Ser Tyr Leu Glu Glu Gln Ala Ala Lys Glu Phe |
| | Ile Ala Trp Leu Val LysGly Arg Gly |
| 211 | His Ala Glu Gly Thr Phe Thr Ser Asp Val Ser Ser Tyr Leu Glu Asp Gln Ala Ala Lys Glu Phe |
| | Ile Ala Trp Leu Val Lys Gly Arg Gly |
| 212 | His Ala Glu Gly Thr Phe Thr Ser Asp Val Ser Ser Tyr Leu Glu Arg Gln Ala Ala Lys Glu Phe |
| | Ile Ala Trp Leu Val Lys Gly Arg Gly |
| 213 | His Ala Glu Gly Thr Phe Thr Ser Asp Val Ser Ser Tyr Leu Glu Lys Gln Ala Ala Lys Glu Phe |
| | Ile Ala Trp Leu Val Lys Gly Arg Gly |
| 214 | His Ala Glu Gly Thr Phe Thr Ser Asp Val Ser Ser Tyr Leu Glu 3-sulfoAla Gln Ala Ala Lys |
| | Glu Phe Ile Ala Trp Leu Val Lys Gly Arg Gly |
| 215 | His Val Glu Gly Thr Phe Thr Ser Asp Val Ser Ser Tyr Leu Glu 3-sulfoAla Gln Ala Ala Lys |
| | Glu Phe Ile Ala Trp LeuVal Lys Gly Arg Gly |
| 216 | His Gly Glu Gly Thr Phe Thr Ser Asp Val Ser Ser Tyr Leu Glu 3-sulfoAla Gln Ala Ala Lys |
| | Glu Phe Ile Ala Trp Leu Val Lys Gly Arg Gly |
| 217 | His Ala Glu Gly Thr Phe Thr Ser Asp Val Ser Ser Tyr Leu Glu Glu Gln Ala Ala Lys Glu Phe |
| | Ile Ala Trp Leu Val Lys Gly Arg |
| 218 | His Ala Glu Gly Thr Phe Thr Ser Asp Val Ser Ser Tyr Leu Glu Asp Gln Ala Ala Lys Glu Phe |
| | Ile Ala Trp Leu Val Lys Gly Arg |
| 219 | His Ala Glu Gly Thr Phe Thr Ser Asp Val Ser Ser Tyr Leu Glu Arg Gln Ala Ala Lys Glu Phe |
| | Ile Ala Trp Leu Val Lys Gly Arg |
| 220 | His Ala Glu Gly Thr Phe Thr Ser Asp Val Ser Ser Tyr Leu Glu Lys Gln Ala Ala Lys Glu Phe |
| | Ile Ala Trp Leu Val Lys Gly Arg |
| 221 | His Ala Glu Gly Thr Phe Thr Ser Asp Val Ser Ser Tyr Leu Glu 3-sulfoAla Gln Ala Ala Lys |
| | Glu Phe Ile Ala Trp Leu Val Lys Gly Arg |
| 222 | His Val Glu Gly Thr Phe Thr Ser Asp Val Ser Ser Tyr Leu Glu Gln Gln Ala Ala Lys Glu Phe |
| | Ile Ala Trp Leu Val Lys Gly Arg |
| 223 | His Val Glu Gly Thr Phe Thr Ser Asp Val Ser Ser Tyr Leu Glu Asp Gln Ala Ala Lys Glu Phe |
| | Ile Ala Trp Leu Val Lys Gly Arg |
| 224 | His Val Glu Gly Thr Phe Thr Ser Asp Val Ser Ser Tyr Leu Glu Arg Gln Ala Ala Lys Glu Phe |
| | Ile Ala Trp Leu Val Lys Gly Arg |
| 225 | His Val Glu Gly Thr Phe Thr Ser Asp Val Ser Ser Tyr Leu Glu Lys Gln Ala Ala Lys Glu Phe |
| | Ile Ala Trp Leu Val Lys Gly Arg |
| 226 | His Val Glu Gly Thr Phe Thr Ser Asp Val Ser Ser Tyr Leu Glu 3-sulfoAla Gln Ala Ala Lys |
| | Glu Phe Ile Ala Trp Leu Val Lys Gly Arg |
| 227 | His Gly Glu Gly Thr Phe Thr Ser Asp Val Ser Ser Tyr Leu Glu Glu Gln Ala Ala Lys Glu Phe |
| | Ile Ala Trp Leu Val Lys Gly Arg |
| 228 | His Gly Glu Gly Thr Phe Thr Ser Asp Val Ser Ser Tyr Leu Glu Asp Gln Ala Ala Lys Glu Phe |
| | Ile Ala Trp Leu Val Lys Gly Arg |
| 229 | His Gly Glu Gly Thr Phe Thr Ser Asp Val Ser Ser Tyr Leu Glu Arg Gln Ala Ala Lys Glu Phe |
| | Ile Ala Trp Leu Val Lys Gly Arg |
| 230 | His Gly Glu Gly Thr Phe Thr Ser Asp Val Ser Ser Tyr Leu Glu Lys Gln Ala Ala Lys Glu Phe |
| | Ile Ala Trp Leu Val Lys Gly Arg |
| 231 | His Gly Glu Gly Thr Phe Thr Ser Asp Val Ser Ser Tyr Leu Glu 3-sulfoAla Gln Ala Ala Lys |
| | Glu Phe Ile Ala Trp Leu Val Lys Gly Arg |
| 232 | His Val Glu Gly Thr Phe Thr Ser Asp Val Ser Ser Tyr Leu Glu Gly Lys Ala Ala Lys Glu Phe |
| | Ile Ala Trp Leu Val Lys Gly Arg Gly |
| 233 | His Val Glu Gly Thr Phe Thr Ser Asp Val Ser Ser Tyr Leu Glu Gly Gln Ala Ala Lys Ala Phe |
| | Ile Ala Trp Leu Val Lys Gly Arg Gly |
| 234 | His Val Glu Gly Thr Phe Thr Ser Asp Val Ser Ser Tyr Leu Glu Gly Gln Ala Ala Lys Glu Phe |
| | Ile Glu Trp Leu Val Lys Gly Arg Gly |
| 235 | His Gly Glu Gly Thr Phe Thr Ser Asp Val Ser Ser Tyr Leu Glu Gly Gln Ala Ala Lys Glu Phe |
| | Ile Glu Trp Leu Val Lys Gly Arg Gly |
| 236 | His Val Gln Gly Thr Phe Thr Ser Asp Val Ser Ser Tyr Leu Glu Gly Gln Ala Ala Lys Glu Phe |
| | Ile Ala Trp Leu Val Lys His Arg Gly |
| 237 | His Val Glu Gly Thr Phe Thr Ser Asp Val Ser Ser Tyr Leu Glu Gly Gln Ala Ala Lys Glu Phe |
| | Ile Ala Trp Leu Val Lys Gly Arg His |
| 238 | His Val Glu Gly Thr Phe Thr Ser Asp Val Ser Ser Tyr Leu Glu Glu Lys Ala Ala Lys Glu Phe |
| | Ile Ala Trp Leu Val Lys Gly Arg Gly |
| 239 | His Val Glu Gly Thr Phe Thr Ser Asp Val Ser Ser Tyr Leu Glu Glu Glu Ala Ala Lys Glu Phe |
| | Ile Ala Trp Leu Val Lys Gly Arg Gly |
| 240 | His Val Glu Gly Thr Phe Thr Ser Asp Val Ser Ser Tyr Leu Glu Glu Gln Ala Ala Lys Ala Phe |
| | Ile Ala Trp Leu Val Lys Gly Arg Gly |
| 241 | His Val Glu Gly Thr Phe Thr Ser Asp Val Ser Ser Tyr Leu Glu Gly Gln Ala Ala Lys Glu Phe |
| | Ile Ala Trp Leu Val Gly Lys Arg Gly |
| 242 | His Val Glu Gly Thr Phe Thr Ser Asp Val Ser Ser Tyr Leu Glu Gly Gln Ala Ala Lys Glu Phe |
| | Ile Ala Trp Leu Val Lys Gly Arg His |
| 243 | His Gly Glu Gly Thr Phe Thr Ser Asp Val Ser Ser Tyr Leu Glu Gly Gln Ala Ala Lys Glu Phe |
| | Ile Ala Trp Leu Val Lys Gly Arg His |

In further embodiments, the bioactive peptide or pritein of the compositions disclosed herein comprise amylin, amylin analogs and amylin derivatives. Any amylin, amylin analogs or amylin deriviatives known in the art can be used in the present compositions, including, but not limited to those disclosed in U.S. Pat. Nos. 6,610,824, 5,686,411, 5,580,953, 5,367,052 and 5,124,314, all of which are incorporated herein by reference in their entireties and in particular the amylin-related sequences described therein. Examples of amylin peptides that may be used are described by the following formula:

Formula VIII
(SEQ ID NO. 248)
A₁-X-Asn-Thr-Ala-Thr-Y-Ala-Thr-Gln-Arg-Leu-B₁-Asn-Phe-Leu-C₁-D₁-E₁-
F₁-G₁-Asn-H₁-Gly-I₁-J₁-Leu-K₁-L₁-Thr-M₁-Val-Gly-Ser-Asn-Thr-Tyr-Z,

A₁ is Lys, Ala, Ser or hydrogen,

B₁ is Ala, Set or Thr;

C₁ is Val, Leu or Ile;

D₁ is His or Arg;

E₁ is Ser or Thr;

F₁ is Ser, Thr, Gln or Asn;

G₁ is Asn, Gin or His;

H₁ is Phe, Leu or Tyr,

I₁ is Ala or Pro;

J₁ is Ile, Val, Ala or Leu;

K₁ is Ser, Pro, Leu, Ile or Thr;

L₁ is Ser, Pro or Thr;

M₁ is Asn, Asp, or Gin;

X and Y are independently selected amino acid residues having side chains which are chemically bonded to each other to form an intramolecular linkage; and
Z is amino, alkylamino, dialkylamino, cycloalkylamino, arylamino, aralkylamino, alkyloxy, aryloxy or aralkyloxy. Particular, but non-limiting examples of amylin analogs and derivatives that can be used are presented in Table 4.

TABLE 4

| SEQ | |
|---|---|
| ID | |
| NO | Sequence |
| 249 | Lys Cys Asn Thr Ala Thr Cys Ala Thr Gln Arg Leu Ala Asn Phe Leu Val His Ser Ser Asn Asn |
| | Phe Gly Ala Ile Leu Ser Ser Thr Asn Val Gly Ser Asn Thr Tyr |
| 254 | Lys Cys Asn Thr Ala Thr Cys Ala Thr Gln Arg Leu Ala Asn Phe Leu Ile Arg Ser Ser Asn Asn |
| | Leu Gly Ala Ile Leu Ser Pro Thr Asn Val Gly Ser Asn Thr Tyr |
| 251 | Lys Cys Asn Thr Ala Thr Cys Ala Thr Gln Arg Leu Ala Asn Phe Leu Val Arg Thr Ser Asn Asn |
| | Leu Gly Ala Ile Leu Ser Pro Thr Asn Val Gly Ser Asn Thr Tyr |
| 252 | Lys Cys Asn Thr Ala Thr Cys Ala Thr Gln Arg Leu Ala Asn Phe Leu Val Arg Ser Ser Asn Asn |
| | Leu Gly Pro Val Leu Pro Pro Thr Asn Val Gly Ser Asn Thr Tyr |
| 253 | Lys Cys Asn Thr Ala Thr Cys Ala Thr Gln Arg Leu Ala Asn Phe Leu Val His Ser Asn Asn Asn |
| | Leu Gly Pro Val Leu Ser Pro Thr Asn Val Gly Ser Asn Thr Tyr |
| 254 | Lys Cys Asn Thr Ala Thr Cys Ala Thr Gln Arg Leu Thr Asn Phe Leu Val Arg Ser Ser His Asn |
| | Leu Gly Ala Ala Leu Leu Pro Thr Asp Val Gly Ser Asn Thr Tyr |
| 255 | Cys Asn Thr Ala Thr Cys Ala Thr Gln Arg Leu Ala Asn Phe Leu Val His Ser Asn Asn Phe |
| | Gly Ala Ile Leu Ser Serr Thr Asn Val Gly Ser Asn Thr Tyr |
| 256 | Lys Cys Asn Thr Ala Thr Cys Ala Thr Gln Arg Leu Ala Asn Phe Leu Val His Ser Ser Asn Asn |
| | Phe Gly Ala Ile Leu Pro Ser Thr Asn Val Gly Ser Asn Thr Tyr |
| 257 | Lys Cys Asn Thr Ala Thr Cys Ala Thr Gln Arg Len Ala Asn Phe Leu Val His Ser Ser Asn Asn |
| | Phe Gly Pro Ile Leu Pro Pro Thr Asn Val Gly Ser Asn Thr Tyr |
| 258 | Lys Cys Asn Thr Ala Thr Cys Ala Thr Gln Arg Leu Ala Asn Phe Leu Val Arg Ser Ser Asn Asn |
| | Phe Gly Pro Ile Leu Pro Ser Thr Asn Val Gly Ser Asn Thr Tyr |
| 259 | Cys Asn Thr Ala Thr Cys Ala Thr Gln Arg Leu Ala Asn Phe Leu Val His Arg Ser Asn Asn Phe |
| | Gly Pro Ile Leu Pro Ser Thr Asn Val Gly Ser Asn Thr Tyr |
| 260 | Lys Cys Asn Thr Ala Thr Cys Ala Thr Gln Arg Leu Ala Asn Phe Leu Val His Ser Ser Asn Asn |
| | Phe Gly Pro Val Leu Pro Pro Thr Asn Val Gly Ser Asn Thr Tyr |
| 261 | Lys Cys Asn Thr Ala Thr Cys Ala Thr Gln Arg Leu Ala Asn Phe Leu Val Arg Ser Ser Asn Asn |
| | Phe Gly Pro Ile Leu Pro Pro Thr Asn Val Gly Ser Asn Thr Tyr |
| 262 | Cys Asn Thr Ala Thr Cys Ala Thr Gln Arg Leu Ala Asn Phe Leu Val Arg Ser Ser Asn Asn Phe |
| | Gly Pro Ile Leu Pro Pro Ser Asn Val Gly Ser Asn Thr Tyr |
| 263 | Cys Asn Thr Ala Thr Cys Ala Thr Gln Arg Leu Ala Asn Phe Leu Val His Ser Ser Asn Asn Phe |
| | Gly Pro Ile Leu Pro Pro Ser Asn Val Gly Ser Asn Thr Tyr |
| 264 | Lys Cys Asn Thr Ala Thr Cys Ala Thr Gln Arg Leu Ala Asn Phe Leu Val His Ser Ser Asn Asn |
| | Leu Gly Pro Val Leu Pro Pro Thr Asn Val Gly Ser Asn Thr Tyr |
| 265 | Lys Cys Asn Thr Ala Thr Cys Ala Thr Gln Arg Leu Ala Asn Phe Leu Val His Ser Ser Asn Asn |
| | Leu Gly Pro Val Leu Pro Ser Thr Asn Val Gly Set Asn Thr Tyr |
| 266 | Cys Asn Thr Ala Thr Cys Ala Thr Gln Arg Leu Ala Asn Phe Leu Val His Ser Ser Asn Asn Leu |
| | Gly Pro Val Leu Pro Ser Thr Asn Val Gly Ser Asn Thr Tyr |
| 267 | Lys Cys Asn Thr Ala Thr Cys Ala Thr Gln Arg Leu Ala Asn Phe Leu Val Arg Ser Ser Asn Asn |
| | Leu Gly Pro Val Leu Pro Ser Thr Asn Val Gly Ser Asn Thr Tyr |
| 268 | Lys Cys Asn Thr Ala Thr Cys Ala Thr Gln Arg Leu Ala Asn Phe Leu Val Arg Ser Ser Asn Asn |
| | Leu Gly Pro Ile Leu Pro Pro Thr Asn Val Gly Ser Asn Thr Tyr |
| 269 | Lys Cys Asn Thr Ala Thr Cys Ala Thr Gln Arg Leu Ala Asn Phe Leu Val Arg Ser Ser Asn Asn |
| | Leu Gly Pro Ile Leu Pro Ser Thr Asn Val Gly Ser Asn Thr Tyr |
| 270 | Lys Cys Asn Thr Ala Thr Cys Ala Thr Gln Arg Leu Ala Asn Phe Leu Ile His Ser Ser Asn Asn |
| | Leu Gly Pro Ile Leu Pro Pro Thr Asn Val Gly Ser Asn Thr Tyr |
| 271 | Lys Cys Asn Thr Ala Thr Cys Ala Thr Gln Arg Leu Ala Asn Phe Leu Val Ile Ser Ser Asn Asn |
| | Phe Gly Pro Ile Leu Pro Pro Thr Asn Val Gly Ser Asn Thr Tyr |
| 272 | Cys Asn Thr Ala Thr Cys Ala Thr Gln Arg Leu Ala Asn Phe Leu Ile His Ser Ser Asn Asn Leu |
| | Gly Pro Ile Leu Pro Pro Thr Asn Val Gly Ser Asn Thr Tyr |
| 273 | Lys Cys Asn Thr Ala Thr Cys Ala Thr Gln Arg Leu Ala Asn Phe Leu Ile Arg Ser Ser Asn Asn |
| | Leu Gly Ala Ile Leu Ser Ser Thr Asn Val Gly Ser Asn Thr Tyr |
| 274 | Lys Cys Asn Thr Ala Thr Cys Ala Thr Gln Arg Leu Ala Asn Phe Leu Ile Arg Ser Ser Asn Asn |
| | Leu Gly Ala Val Leu Ser Pro Thr Asn ValGly Ser Asn Thr Tyr |
| 275 | Lys Cys Asn Thr Ala Thr Cys Ala Thr Gln Arg Leu Ala Asn Phe Leu Ile Arg Ser Ser Asn Asn |
| | Leu Gly Pro Val Leu Pro Pro Thr Asn Val Gly Ser Asn Thr Tyr |
| 276 | Lys Cys Asn Thr Ala Thr Cys Ala Thr Gln Arg Leu Thr Asn Phe Leu Val His Ser Ser His Asn |
| | Leu Gly Ala Ala Leu Leu Pro Asp Val Gly Ser Asn Thr Tyr |
| 277 | Lys Cys Asn Thr Ala Thr Cys Ala Thr Gln Arg Leu Thr Asn Phe Leu Val His Ser Ser His Asn |
| | Leu Gly Ala Ala Leu Ser Pro Thr Asp Val Gly Ser Asn Thr Tyr |
| 278 | Cys Asn Thr Ala Thr Cys Ala Thr Gln Arg Leu Thr Asn Phe Leu Val His Ser Ser His Asn Leu |
| | Gly Ala Val Leu Pro Ser Thr Asp Val Gly Ser Asn Thr Tyr |
| 279 | Lys Cys Asn Thr Ala Thr Cys Ala Thr Gln Arg Leu Thr Asn Phe Leu Val Arg Ser Ser His Asn |
| | Leu Gly Ala Ala Leu Ser Pro Thr Asp Val Gly Ser Asn Thr Tyr |
| 280 | Lys Cys Asn Thr Ala Thr Cys Ala Thr Gln Arg Leu Thr Asn Phe Leu Val Arg Ser Ser His Asn |
| | Leu Gly Ala Ile Leu Pro Pro Thr Asp Val Gly Ser Asn Thr Tyr |
| 281 | Lys Cys Asn Thr Ala Thr Cys Ala Thr Gln Arg Leu Thr Asn Phe Leu Val Arg Ser Ser His Asn |
| | Leu Gly Pro Ala Leu Pro Pro Thr Asp Val Gly Ser Asn Thr Tyr |
| 282 | Lys Asp Asn Thr Ala Thr Lys Ala Thr Gln Arg Leu Ala Asn Phe Leu Val His Ser Ser Asn Asn |
| | Phe Gly Ala Ile Leu Ser Ser Thr Asn Val Gly Ser Asn Thr Tyr |
| 283 | Ala Cys Asn Thr Ala Thr Cys Ala Thr Gln Arg Leu Ala Asn Phe Leu Val His Ser Ser Asn Asn |
| | Phe Gly Ala Ile Leu Set Ser Thr Asn Val Gly Ser Asn Thr Tyr |
| 284 | Ser Cys Asn Thr Ala Thr Cys Ala Thr Gln Arg Leu Ala Asn Phe Leu Val His Ser Ser Asn Asn |
| | Phe Gly Ala Ile Leu Ser Ser Thr Asn Val Gly Ser Asn Thr Tyr |
| 285 | Lys Cys Asn Thr Ala Thr Cys Ala Thr Gln Arg Leu Ala Asn Phe Leu Val His Ser Ser Asn Asn |
| | Phe Gly Ala Ile Leu Ser Pro Thr Asn Val Gly Ser Asn Thr Tyr |
| 286 | Lys Cys Asn Thr Ala Thr Cys Ala Thr Gln Arg Leu Ala Asn Phe Leu Val His Ser Ser Asn Asn |
| | Phe Gly Pro Ile Leu Pro Ser Thr Asn Val Gly Ser Asn Thr Tyr |
| 287 | Cys Asn Thr Ala Thr Cys Ala Thr Gln Arg Leu Ala Asn Phe Leu Val His Ser Ser Asn Asn Phe |
| | Gly Pro Ile Leu Pro Ser Thr Asn Val Gly Ser Asn Thr Tyr |
| 288 | Cys Asn Thr Ala Thr Cys Ala Thr Gln Arg Leu Ala Asn Phe Leu Val His Ser Ser Asn Asn Phe |
| | Gly Pro Val Leu Pro Pro Ser Asn Val Gly Ser Asn Thr Tyr |
| 289 | Lys Cys Asn Thr Ala Thr Cys Ala Thr Gln Arg Leu Ala Asn Phe Leu Val His Ser Ser Asn Asn |
| | Phe Gly Ala Ile Leu Ser Ser Thr Asn Val Gly Ser Asn Thr Tyr |
| 290 | Lys Cys Asn Thr Ala Thr Cys Ala Thr Gln Arg Leu Ala Asn Phe Leu Val His Ser Ser Asn Asn |
| | Phe Gly Pro Ile Leu Pro Pro Thr Asn Val Gly Ser Asn Thr Tyr |
| 291 | Lys Cys Asn Thr Ala Thr Cys Val Leu Gly Arg Leu Ser Gln Glu Leu His Arg Leu Gln Thr Tyr |
| | Pro Arg Thr Asn Thr Gly Ser Asn Thr Tyr NH₂ |
| 292 | Cys Ser Asn Leu Ser Thr Cys Val Leu Gly Arg Leu Ser Gln Glu Leu His Arg Leu Gln Thr Tyr |
| | Pro Arg Thr Asn Thr Gly Ser Ans Thr Tyr NH₂ |

Included in the compositions and methods disclosed herein are analogs and derivatives of bioactive peptides or proteins that have undergone one or more amino acid substitutions, additions or deletions. In one embodiment, the analog or derivative has undergone not more than 10 amino acid substitutions, deletions and/or additions. In another embodiment, the analog or derivative has undergone not more than 5 amino acid substitutions, deletions and/or additions.

Substitutions of amino acids within a peptide or protein while retaining at least one of the biological activities associated with the parent peptide or protein is known within the art of protein chemistry. It is recognized in the art that modifications in the amino acid sequence of a peptide, polypeptide, or protein can result in equivalent, or possibly improved, second generation peptides, etc., that display equivalent or superior functional characteristics when compared to the original amino acid sequence. Alterations can include amino acid insertions, deletions, substitutions, truncations, fusions, shuffling of subunit sequences, and the like.

One factor that can be considered in making such changes is the hydropathic index of amino acids. The importance of the hydropathic amino acid index in conferring interactive biological function on a protein has been discussed by Kyte and Doolittle (J. Mol. Biol., 157: 105-132, 1982). It is accepted that the relative hydropathic character of amino acids contributes to the secondary structure of the resultant protein.

Based on its hydrophobicity and charge characteristics, each amino acid has been assigned a hydropathic index as follows: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate/glutamine/aspartate/asparagine (−3.5); lysine (−3.9); and arginine (−4.5).

As is known in the art, certain amino acids in a peptide or protein can be substituted for other amino acids having a similar hydropathic index or score and produce a resultant peptide or protein having similar biological activity, i.e., which still retains biological functionality. In making such changes, it is preferable that amino acids having hydropathic indices within ±2 are substituted for one another. More preferred substitutions are those wherein the amino acids have hydropathic indices within ±1. Most preferred substitutions are those wherein the amino acids have hydropathic indices within ±0.5.

Like amino acids can also be substituted on the basis of hydrophilicity. U.S. Pat. No. 4,554,101 discloses that the greatest local average hydrophilicity of a protein, as governed by the hydrophilicity of its adjacent amino acids, correlates with a biological property of the protein. The following hydrophilicity values have been assigned to amino acids: arginine/lysine (+3.0); aspartate/glutamate (+3.0±1); serine (+0.3); asparagine/glutamine (+0.2); glycine (0); threonine (−0.4); proline (−0.5±1); alanine/histidine (−0.5); cysteine (−1.0); methionine (−1.3); valine (−1.5); leucine/isoleucine (−1.8); tyrosine (−2.3); phenylalanine (−2.5); and tryptophan (−3.4). Thus, one amino acid in a peptide, polypeptide, or protein can be substituted by another amino acid having a similar hydrophilicity score and still produce a resultant protein having similar biological activity, i.e., still retaining correct biological function. In making such changes, amino acids having hydrophilicity values within ±2 are preferably substituted for one another, those within ±1 are more preferred, and those within ±0.5 are most preferred.

As outlined above, amino acid substitutions in the bioactive peptides and proteins for use in the compositions and methods disclosed herein can be based on the relative similarity of the amino acid side-chain substituents, for example, their hydrophobicity, hydrophilicity, charge, size, etc. Exemplary substitutions that take various of the foregoing characteristics into consideration in order to produce conservative amino acid changes resulting in silent changes can be selected from other members of the class to which the naturally occurring amino acid belongs. Amino acids can be divided into the following four groups: (1) acidic amino acids; (2) basic amino acids; (3) neutral polar amino acids; and (4) neutral non-polar amino acids. Representative amino acids within these various groups include, but are not limited to: (1) acidic (negatively charged) amino acids such as aspartic acid and glutamic acid; (2) basic (positively charged) amino acids such as arginine, histidine, and lysine; (3) neutral polar amino acids such as glycine, serine, threonine, cysteine, cystine, tyrosine, asparagine, and glutamine; and (4) neutral non-polar amino acids such as alanine, leucine, isoleucine, valine, proline, phenylalanine, tryptophan, and methionine. It should be noted that changes which are not expected to be advantageous can also be useful if these result in the production of functional sequences.

Also included within the scope of the bioactive peptides and proteins that can be used in the present compositions are conjugates of the above referenced proteins, peptides and peptide analogs, e.g., chemically modified with or linked to at least one molecular weight enhancing compound known in the art such as polyethylene glycol, and chemically modified equivalents of such proteins, peptides, analogs, or conjugates. The polyethylene glycol polymers may have molecular weights between about 500 Da and 20,000 Da. Preferred conjugates include those described in International Patent Publication No. WO 00/66629, which is herein incorporated by reference in its entirety. In one embodiment, the bioactive peptides and proteins of the invention have a molecular weight up to about 100,000 Da, in another embodiment up to about 25,000 Da, while in still another embodiment up to about 5,000 Da.

As used herein, the terms “protein” or “peptide” include any molecule that comprises five or more amino acids. It is well known in the art that proteins may undergo modification, including post-translational modifications, such as, but not limited to, disulfide bond formation, glycosylation, phosphorylation, or oligomerization. Thus, as used herein, the term “protein” or “peptide” includes any protein or peptide that is modified by any biological or non-biological process.

The term “amino acid” is used in its broadest sense, and includes naturally occurring amino acids as well as non-naturally occurring amino acids, including amino acid analogs and derivatives. The latter includes molecules containing an amino acid moiety. One skilled in the art will recognize, in view of this broad definition, that reference herein to an amino acid includes, for example, naturally occurring proteogenic L-amino acids; D-amino acids; chemically modified amino acids such as amino acid analogs and derivatives; naturally occurring non-proteogenic amino acids such as norleucine, β-alanine, ornithine, norvaline, homocysteine, homoserine etc.; and chemically synthesized compounds having properties known in the art to be characteristic of amino acids. As used herein, the term “proteogenic” indicates that the amino acid can be incorporated into a peptide, polypeptide, or protein in a cell through a metabolic pathway.

The term “polyamino acid” refers to any homopolymer or mixture of homopolymers of a particular amino acid. The amino acids in a polyamino acid can be any amino acid including L-amino acids, D-amino acids or a combination of D- and L amino acids.

As used herein in reference to a peptide or protein, the term “derivative” means a protein or peptide that is obtained by modification of a parent protein or peptide, for example, by amino acid substitution, addition or deletion. In one embodiment, derivatives have at least 15% sequence identity to the parent molecule. In other embodiments, derivatives have at least 50%, at least 70%, at least 80%, at least 90% or at least 95% sequence identity with the parental protein or peptide.

As used herein “analog” refers to bioactive peptides or proteins that are structurally related to a parent peptide or protein by amino acid sequence but which differ from the parent in a characteristic of interest such as bioactivity, solubility, resistance to proteolysis, etc. In certain embodiments, analogs have activities between about 1% to about 10,000%, about 10% to about 1000%, and about 50% to about 500% of the bioactivity of the parental protein or peptide.

The term “bioactive” or “bioactivity” means the ability to affect any physical or biochemical properties of a biological organism, including but not limited to viruses, bacteria, fungi, plants, animals, and humans. In particular, as used herein, bioactive includes diagnosis, cure, mitigation, treatment, or prevention of disease in humans or other animals, or to otherwise enhance physical or mental well-being of humans or animals.

As used herein “subject” or “patient” refers to any animal including domestic animals such as domestic livestock and companion animals. The terms are also meant to include human beings.

The cationic polyamino acids of the invention include polymers of basic amino acids, such as histidine, arginine, and lysine, that are protonated in a neutral or acidic pH environment and are thus cationic. The molecular weight of such polymers, e.g., poly-L-histidine, poly-L-arginine, poly-L-lysine, or copolymers thereof, are generally between about 10 and about 300 kDa. In another embodiment, the polymers have an average molecular weight of between about 100 kDa and about 200 kDa. In still a further embodiment, the polymers have an average molecular weight between about 140 kDa and about 150 kDa, while in yet another embodiment the polymers have an average molecular weight of between about 140 kDa and about 200 kDa. In one particular embodiment the cationic polyamino acid of the composition is poly-L-arginine hydrochloride with an average molecular weight of about 141 kDa.

Buffers useful in connection with the compositions and methods disclosed herein can be any buffer that displays adequate buffering capacity (buffer value) at the pH ranges which render the bioactive peptides and proteins of the invention chemically stable for the duration of use, and which are physically compatible with the cationic polyamino acids of the invention at the concentrations and pHs of use, i.e., they do not cause precipitation of the cationic polyamino acid. Methods for calculating the buffering capacity (buffer value) of a buffer at a particular concentration and pH are well known in the art and can be determined by the skilled artisan without undue experimentation.

It has been found that traditional buffer components with multi-anionic charges such as citric acid generally are not physically compatible with the cationic polyamino acids of the invention, resulting in precipitation of the polyamino acid. However, buffer components containing neutral and mono-anionic net charges are compatible with, and can be used in combination with the cationic polyamino acids of the invention. Examples of suitable buffers include, but are not limited to, acetic acid, ε-aminocaproic acid, and glutamic acid.

The pharmaceutical compositions of the invention may further comprise any number of known pharmaceutically acceptable excipients such as, but not limited to, tonicifying agents, viscosity-increasing agents, bioadhesive agents, preservatives, diluents, carriers, and the like.

Examples of tonicifying agents that may be used, include, but are not limited to, sodium chloride, mannitol, sucrose, and glucose. However, any tonicifying agent known in the art to prevent mucosal irritation can be used. Other compounds that can be included in the compositions include lactose, sorbitol, trehalose, sucrose, mannose, maltose, and derivatives and homologs thereof.

Exemplary viscosity-increasing and bioadhesive agents that may be used in the compositions disclosed herein, include, but are not limited to, cellulose derivatives (e.g., hydroxypropyl cellulose, hydroxypropyl methylcellulose or methylcellulose of average molecular weight between 10 and 1,500 kDa), starch, gums, carbomers, and polycarbophil. However, any viscosity-increasing or bioadhesive agents known in the art to afford a higher viscosity or to increase the residence time of the pharmaceutical composition at the absorption site may be used.

The compositions may also comprise a surface active agent. Examples of surface active agents or surfactants that may be used include, but are not limited to, polysorbate 20 (Tween 20), polsorbate 80 (Tween 80), polyethylene glycol (PEG), cetyl alcohol, polyvinylpyrolidone (PVP), polyvinyl alcohol (PVA), lanolin alchold. Sorbitan monooleate, and didecanoyl phosphatidylcholine (DDPC).

Additional agents that can be used in combination with cationic polyamino acids to enhance permeability are the cyclodextrins. Any cyclodextrin can be used including alpha-, beta- and gamma-cyclodextrins and any derivative thereof such as methyl-beta-cyclodextran. Examples of other compounds that can be used include hydroxypropyl-beta-cyclodextran, sulfobutyether-beta-cyclodextran and chitosan.

In some embodiments, the composition also includes a chelating agent. Any suitable chelating agent known in the art can be used. Specific examples of chelating agents include ethylene diamine tetraacetic acid (EDTA) and ethylene glycol tetraacetic acid (EGTA).

With the availability of preservative-free spray systems to the pharmaceutical industry, the incorporation of preservative(s) becomes optional in the composition of this invention. Should a preservative system be required or desired, preservative(s) may be added such as benzalkonium chloride, phenylethyl alcohol, methylparaben, ethylparaben, propylparaben, butylparaben, chlorobutanol, benzoic acid, sorbic acid, phenol, m-cresol and alcohol.

The compositions of the present invention can further comprise aqueous carriers, non-aqueous carriers or suspension media. For instance, the pharmaceutical compositions of the invention may be formulated as an aqueous solution in purified water, or may be dispersed in non-aqueous media to thereby be compatible with aerosolization or delivery by instillation in non-aqueous suspension media. By way of example, such non-aqueous suspension media can include hydrofluoroalkanes, fluorocarbons, perfluorocarbons, fluorocarbon/hydrocarbon diblocks, hydrocarbons, alcohols, ethers, and combinations thereof. However, it is understood that any non-aqueous suspension media known in the art may be used in conjunction with the compositions and method disclosed herein.

As mentioned above, the pharmaceutical compositions of the invention may be formulated in a variety of dosage forms suitable for transmucosal delivery, as known in the art. For instance, the compositions may be formulated as an aqueous solution or suspension, a non-aqueous solution or suspension, a tablet, or a dry powder. In one embodiment, the composition is provided in freeze-dried or lyophilized form and reconstituted prior to use. In any event, the compositions of the invention will generally comprise a therapeutically or prophylactically effective amount of a bioactive peptide or protein and an absorption enhancing amount of a mixture comprising a cationic polyamino acid and a buffer that is compatible with the cationic polyamino acid.

One embodiment provides a pharmaceutical composition for nasal delivery in the form of an aqueous solution with enhanced transmucosal absorption, wherein the pharmaceutical composition includes a bioactive peptide or protein; an absorption enhancing cationic polyamino acid; a buffer that is compatible with said cationic polyamino acid; and a bioadhesive agent. Another embodiment of the invention provides a pharmaceutical composition for sublingual delivery in the form of a tablet.

In one embodiment, the weight ratio of bioactive peptide or protein to cationic polyamino acid in the final formulation ranges from 1:100 to 100:1, in another embodiment from 1:25 to 25:1, in yet another embodiment from 1:10 to 10:1, and in still yet another embodiment from 1:2 to 2:1.

The weight ratio of cationic polyamino acid to buffer can vary widely and may be determined by routine experimentation. The only limitation is that adequate buffer is included such that the cationic polyamino acid does not precipitate in the formulated dosage form or upon administration to the desired mucous membrane. In one embodiment the useful weight ratios of cationic polyamino acid to buffer range from 1:100 to 100:1, while in another embodiment the weight ratio of cationic polyamino acid to buffer ranges from 1:25 to 25:1. In other embodiments, the weight ratio of cationic polyamino acid to buffer ranges from 1:10 to 10:1, and from 1:2 to 2:1

When formulated as an aqueous solution, the instant pharmaceutical compositions may comprise, for example, 0.01%-5.0% (w/v) of the bioactive peptide or protein; 0.01%-1.0% (w/v) of the cationic polyamino acid; 0.01%-10.0% (w/v) of the buffer, 0.001%-10.0% (w/v) of the optional tonicifying agent; 0.001%-10.0% (w/v) of the optional viscosity-increasing agent; 0.001%-10.0% (w/v) of the optional bioadhesive agent; 0.001%-10.0% (w/v) of the optional preservative; q.s. (quantum sufficiat) to 100.0% (w/v) of purified water;

The term “therapeutically or prophylactically effective amount” as used herein refers to an amount of a bioactive peptide or protein to treat, ameliorate, or prevent a disease or condition of interest, or to exhibit a detectable therapeutic or preventative effect. The effect can be detected by, for example, a reduction of plasma glucose or HbA_{1c} levels, or reduction or maintenance of body weight. Therapeutic effects also include reduction in physical symptoms. The precise effective amount for a subject will depend upon the subject's size and health, the nature and extent of the condition, and the therapeutics or combination of therapeutics selected for administration. Generally, the effective amount for a given situation can be determined by routine experimentation and is within the judgement of the clinician.

The exact dosage will be determined by the practitioner, in light of factors related to the subject that requires treatment. Dosage and administration are adjusted to provide sufficient levels of the active moiety or to maintain the desired effect. Factors that may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the active ingredient in the particular formulation.

For any compound, the therapeutically effective dose can be estimated initially either in cell culture assays, e.g., of neoplastic cells, or in animal models, usually mice, rats, rabbits, dogs, pigs, or primates. The animal model may also be used to determine the appropriate concentration range and route of administration. Such information can then be used to determine useful doses and routes for administration in humans. Further, therapeutic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between therapeutic and toxic effects is the therapeutic index, and it can be expressed as the ratio, ED₅₀/LD₅₀. Pharmaceutical compositions which exhibit large therapeutic indices are preferred. The data obtained from cell culture assays and animal studies is used in formulating a range of dosage for human use. The dosage contained in such compositions is preferably within a range of circulating concentrations that include the ED₅₀ with little or no toxicity. The dosage varies within this range depending upon the dosage form employed, sensitivity of the patient, and the route of administration.

The term “absorption enhancing amount” as used herein refers to an amount of the absorption enhancing mixture such that the transmucosal absorption of the bioactive peptide or protein is enhanced by at least 1.5-fold, at least 2-fold, at least 5-fold, or at least 10-fold, as compared to transmucosal absorption of the bioactive peptide or protein in the absence or substantial absence of the absorption enhancing mixture. Generally, an effective absorption enhancing amount for a given situation can be determined by routine experimentation.

In one embodiment, the pharmaceutical composition is formulated as an aqueous solution and includes: exendin-4; poly-L-arginine of average molecular weight between 10 and 300 kDa; glutamate buffer at pH between 4.0 and 5.0; sodium chloride; and purified water. In another embodiment, the pharmaceutical composition includes: exendin-4; poly-L-arginine of average molecular weight between 10 and 300 kDa; glutamate buffer at pH between 4.0 and 5.0; sodium chloride; hydroxypropyl methylcellulose of average molecular weight between 10 kDa and 1,500 kDa; and purified water.

In a further embodiment, the pharmaceutical composition may include exendin-4 at a concentration between 0.01% and 5.0% (w/v); poly-L-arginine of average molecular weight between 10 kDa and 300 kDa at a concentration between 0.01% and 1.0% (w/v); glutamate buffer at pH between 4.0 and 5.0 at a concentration between 0.01% and 10.0% (w/v); sodium chloride at a concentration between 0.001% and 0.9% (w/v); and purified water to 100%.

In another embodiment, the pharmaceutical composition includes exendin-4 at a concentration between 0.01% and 5.0% (w/v); poly-L-arginine of average molecular weight between 10 kDa and 300 kDa at a concentration between 0.01% and 1.0% (w/v); glutamate buffer at pH between 4.0 and 5.0 at a concentration between 0.01% and 10.0% (w/v); sodium chloride at a concentration between 0.001% and 0.9% (w/v); hydroxypropyl methylcellulose of average molecular weight 10 kDa and 1,500 kDa at a concentration between 0.001% and 10.0% (w/v); and purified water to make 100%.

In yet another embodiment of the invention, the pharmaceutical composition includes exendin-4 at a concentration of 0.5% to 1.0% (w/v); poly-L-arginine hydrochloride of average molecular weight 141 kDa at a concentration of 0.5% (w/v); glutamate buffer at pH 4.5 at a concentration of 0.56% (w/v); sodium chloride at a concentration of 0.72% (w/v); and purified water to 100%.

In another embodiment, the pharmaceutical composition of the invention may include exendin-4 at a concentration of 0.5% to 1.0% (w/v); poly-L-arginine hydrochloride of average molecular weight of 141 kDa at a concentration of 0.5% (w/v); glutamate buffer at pH of 4.5 at a concentration of 0.56% (w/v); sodium chloride at a concentration of 0.72% (w/v); hydroxypropyl methylcellulose of average molecular weight ranging from about 4 to about 86 kDa at a concentration 0.5% (w/v); and purified water to 100%.

Any of the above embodiments can be supplemented with any additional absorption enhancing agent known in the art, such as those described herein, including chitosan, phospholipids, cyclodextrins, surfactants, and any combination or mixture thereof.

In one aspect, the compositions disclosed herein can be formulated for transmucosal delivery to or via the mucous membranes of a patient in need of treatment. Such formulations can be delivered to or via the mucous membranes for prophylactic or therapeutic purposes in any manner known in the art such as, but not limited to, drops, sprays, tablets, dry-powder inhalation, instillation, metered dose inhalation, nebulization, aerosolization, or instillation as suspension in compatible vehicles. More particularly, ocular, nasal, pulmonary, buccal, sublingual, rectal, or vaginal administration is contemplated as within the scope of the invention.

In one embodiment, the pharmaceutical composition may be administered as an aqueous solution in the form of drops or a spray. In another embodiment, the pharmaceutical composition disclosed herein may be administered as a dry powder formulation. In yet another embodiment, the pharmaceutical composition may be administered as a tablet formulation, wherein the tablet preferably comprises a bioadhesive agent.

The compositions disclosed herein may also be administered via aerosolization, such as with a dry powder inhaler (DPI), metered dose inhaler (MDI), liquid dose instillation (LDI), and nebulizers. DPIs, MDIs, LDIs, and nebulizers are all well known in the art and could easily be employed for administration of the pharmaceutical compositions of the invention without undue experimentation.

In another aspect, a method for enhancing the transmucosal absorption of a bioactive peptide or protein is provided, wherein the method involves administering the bioactive peptide or protein to a subject via a mucous membrane in conjunction with an absorption enhancing composition comprising a cationic polyamino acid and a buffer that is compatible with that cationic polyamino acid.

Generally stated, the transmucosal absorption of the bioactive peptide or protein is enhanced relative to the transmucosal absorption of the bioactive peptide or protein in the absence or substantial absence of the absorption enhancing composition comprising a cationic polyamino acid. In one embodiment, the transmucosal absorption of the bioactive peptide or protein is improved by at least 1.5-fold, at least 2-fold, in another embodiment at least 5-fold, and in still another embodiment by at least 10-fold over the transmucosal absorption of the bioactive peptide or protein when administered to a subject via transmucosal delivery in the absence or the substantial absence of the absorption enhancing composition.

In one embodiment, the bioactive peptide or protein is administered as an aqueous solution comprising the absorption enhancing composition. In another embodiment, the bioactive peptide or protein is administered as a dry powder formulation comprising the absorption enhancing composition. In yet another embodiment, the bioactive peptide or protein is administered as a tablet formulation comprising the absorption enhancing composition, wherein the absorption enhancing composition optionally further comprises a bioadhesive agent.

Another aspect relates to a method for improving the bioavailability of a bioactive peptide or protein administered to a subject via transmucosal delivery, wherein the method generally involves administering the bioactive peptide or protein to a subject via a mucous membrane in conjunction with an absorption enhancing composition comprising a cationic polyamino acid and a buffer that is compatible with that cationic polyamino acid. According to one embodiment of the method, the bioavailability of the bioactive peptide or protein is improved by at least 15-fold, at least 2-fold, in another embodiment of the invention at least 5-fold, and in yet another embodiment of the method by at least 10-fold over the bioavailability of the bioactive peptide or protein when administered to a subject via transmucosal delivery in the absence or substantial absence of the absorption enhancing composition.

The following examples are intended to provide illustrations of the application of the present invention. The following examples are not intended to completely define or otherwise limit the scope of the invention.

### EXAMPLES

The peptide exendin-4 (AC2993) is useful as a model for peptides or proteins with iso-electric points that lend themselves (or can be buffered) to have either neutral or positive net charges within the pH range from about 4 to about 7 for optimum transmucosal delivery.

### Example 1

An aqueous pharmaceutical composition was prepared as follows: 0.5% weight by volume of exendin-4; 0.5% weight by volume of poly-L-arginine hydrochloride of average molecular weight 141 kDa; 0.56% weight by volume of monosodium glutamate, monohydrate; 0.72% weight by volume of sodium chloride; hydrochloric acid q.s. to adjust the pH to approximately 4.5; q.s. to 100.0% weight by volume of water.

### Example 2

An aqueous pharmaceutical composition was prepared as follows: 0.5% weight by volume of exendin-4; 0.25% weight by volume of poly-L-arginine hydrochloride of average molecular weight 141 kDa; 0.56% weight by volume of monosodium glutamate, monohydrate; 0.72% weight by volume of sodium chloride; hydrochloric acid q.s. to adjust the pH to approximately 4.5; q.s. to 100.0% weight by volume of water.

### Example 3

An aqueous pharmaceutical composition was prepared as follows: 0.5% weight by volume of exendin-4; 0.5% weight by volume of poly-L-arginine hydrochloride of average molecular weight 141 kDa; 0.56% weight by volume of monosodium glutamate, monohydrate; 0.72% weight by volume of sodium chloride; 0.5% weight by volume of hydroxypropyl methylcellulose of average molecular weight approximately 86 kDa; hydrochloric acid q.s. to adjust the pH to approximately 4.5; q.s. to 100.0% weight by volume of water.

### Example 4

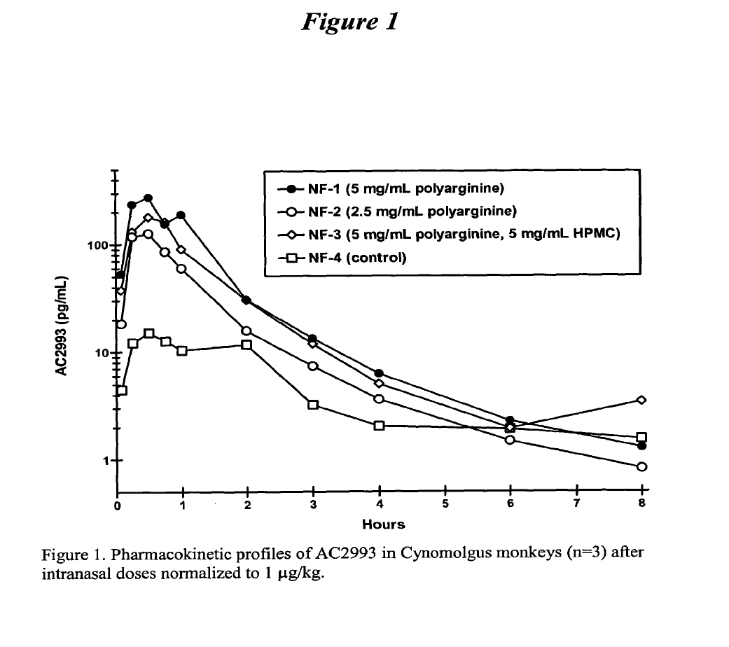

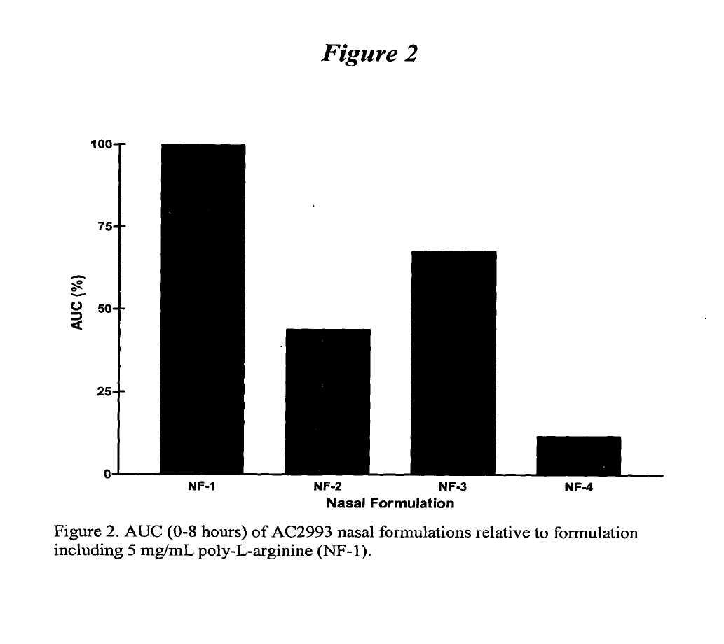

To evaluate the efficacy of the transmucosal absorption enhancing ability of the cationic polyamino acids of the invention, the aqueous pharmaceutical compositions of Examples 1-3, and a control composition (prepared in the absence of the cationic polyamino acid) were prepared and nasally administered to Cynomolgus monkeys via a spray bottle. As depicted in FIGS. 1 and 2, the presence of a cationic polyamino acid (poly-L-arginine) showed a significant, concentration dependent effect on transmucosal absorption and bioavailability which was dependent on the concentration of the polyamino acid. More specifically, FIG. 1 depicts the bioavailability enhancement (normalized to a 1 μg/kg dose) of three exendin-4 aqueous solutions containing poly-L-arginine with or without hydroxypropyl methylcellulose as compared to a control exendin-4 solution without poly-L-arginine. FIG. 2 depicts the area under the plasma curves (AUC) up to 8 hours post-dosing of the exendin-4 solutions relative to the solution affording the highest bioavailability (NF-1). The data show that the AUC of the exendin-4 control solution without poly-L-arginine (NF-4) is approximately one-tenth of that of the solution containing 0.5% poly-L-arginine (NF-1). Thus, the bioavailability is unexpectedly enhanced 10-fold by the poly-L-arginine formulation.

### CONCLUSION

In light of the detailed description of the invention and the examples presented above, it can be appreciated that the several aspects of the invention are achieved.

It is to be understood that the present invention has been described in detail by way of illustration and example in order to acquaint others skilled in the art with the invention, its principles, and its practical application. Particular formulations and processes of the present invention are not limited to the descriptions of the specific embodiments presented, but rather the descriptions and examples should be viewed in terms of the claims that follow and their equivalents. While some of the examples and descriptions above include some conclusions about the way the invention may function, the inventors do not intend to be bound by those conclusions and functions, but put them forth only as possible explanations.

It is to be further understood that the specific embodiments of the present invention as set forth are not intended as being exhaustive or limiting of the invention, and that many alternatives, modifications, and variations will be apparent to those of ordinary skill in the art in light of the foregoing examples and detailed description. Accordingly, this invention is intended to embrace all such alternatives, modifications, and variations that fall within the spirit and scope of the following claims.

1-61. (canceled)

62. A pharmaceutical composition for transmucosal administration, the composition comprising:
(i) about 0.5% to about 1.0% (w/v) of exendin-4;
(ii) about 0.01% to about 1.0% (w/v) of a cationic polyamino acid selected from poly-histidine, poly-arginine, poly-lysine, or a combination of two or more thereof;
(iii) at least one additional absorption enhancing agent selected from the group consisting of a chitosan, a phospholipid, a cyclodextrin, a surfactant, or a combination of two or more thereof; and
(iv) about 0.01% to about 10.0% (w/v) of a compatible buffer selected from acetic acid, aspartic acid, ε-aminocaproic acid, glutamic acid, or a combination of two or more thereof, wherein the compatible buffer has a mono-anionic or neutral net charge; wherein the pH of the composition is 3.0 to 8.0; and wherein the transmucosal absorption of exendin-4 is increased relative to the absorption of exendin-4 in the absence of poly-histidine, poly-arginine, poly-lysine, or a combination of two or more thereof, and wherein the cationic polyamino acid does not precipitate in the composition and upon administration to a mucous membrane.

63. The composition of claim 62, further comprising sodium chloride, mannitol, sucrose, glucose, or a combination of two or more thereof.

64. The composition of claim 62, further comprising hydroxypropyl cellulose, hydroxypropyl methylcellulose, a methylcellulose of average molecular weight between about 10 and about 1,500 kDa, starch, a gum, or a combination of two or more thereof

65. The composition of claim 62, further comprising a carbomer, a polycarbophil, or a combination thereof.

66. The composition of claim 62, further comprising benzalkonium chloride, phenylethyl alcohol, methylparaben, ethylparaben, propylparaben, butylparaben, chlorobutanol, benzoic acid, sorbic acid, phenol, m-cresol, alcohol, or a combination of two or more thereof.

67. The composition of claim 62, wherein the absorption is increased at least 2-fold.

68. The composition of claim 62, wherein the absorption is increased at least 5-fold.

69. The composition of claim 62, wherein the absorption is increased at least 10-fold.

70. The composition of claim 62, wherein said absorption is increased at least 1.5-fold.

71. The composition of claim 62, wherein the pH of said composition is between pH 4.0 and pH 6.0.

72. The composition of claim 62, wherein the pH of said composition is between pH 4.0 and pH 5.0.

73. The composition of claim 62, further comprising between about 0.001% to about 10.0% of a tonicifying agent.

74. The composition of claim 62, further comprising between about 0.001% to about 10.0% of a viscosity-increasing agent.

75. The composition of claim 62, further comprising between about 0.001% to about 10.0% of a bioadhesive agent.

76. The composition of claim 62, further comprising between about 0.001% to about 10.0% of a preservative.

77. A method for transmucosal administration of exendin-4, the method comprising contacting a mucosal surface with the composition of claim 1 for a time sufficient for a therapeutically effective amount of said exendin-4 to pass through the mucosal surface.
